(19) United States
(12) Patent Application Publication
(10) Pub. No.: US 2022/0050096 A1
(43) Pub. Date: Feb. 17, 2022

(54) ANALYTE-SENSITIVE PROBES AND CONTACT LENS FOR DIAGNOSIS OF OCULAR PATHOLOGIES

(71) Applicant: University Of Maryland, Baltimore, Baltimore, MD (US)

(72) Inventors: Joseph R. Lakowicz, Ellicot City, MD (US); Ramachandram Badugu, Ellicott City, MD (US); E. Albert Reece, Lutherville, MD (US)

(21) Appl. No.: 17/402,092

(22) Filed: Aug. 13, 2021

Related U.S. Application Data
(60) Provisional application No. 63/065,540, filed on Aug. 14, 2020.
(60) Provisional application No. 63/065,562, filed on Aug. 14, 2020.

(51) Int. Cl. G01N 33/52; A61B 5/145; A61B 5/00

(52) U.S. Cl. CPC G01N 33/52; A61B 5/14546; G01N 2800/16; A61B 5/14507; A61B 2562/0233; A61B 5/6821

(57) ABSTRACT

A probe compound, contact lens containing bound probe, and method are disclosed for use in detecting analytes in basal tears on the surface of the eye of a subject. The probe composition preferably includes a hydrophilic portion, an analyte-sensitive portion and a fluorophore portion as well as a hydrophobic portion that allows binding to the contact lens material and optionally can modify the binding affinity of the fluorophore probe. The analyte-sensitive portion is configured to bind to a specific analyte in aqueous solution or to be quenched by the analyte. The fluorophore portion is configured to change an optical property of fluorescent light emitted in response to incident excitation light when the probe composition changes between a first state in which the analyte is not bound to the analyte-binding portion and a second state in which the analyte binds to the analyte-binding portion.

### CROSS-REFERENCE TO RELATED APPLICATIONS

This application claims the benefit of U.S. provisional application Ser. No. 63/065,540, filed 14 Aug. 2020 and U.S. provisional application Ser. No. 63/065,562, filed 14 Aug. 2020. The entire contents of these applications are hereby incorporated by reference as if fully set forth herein.

### GOVERNMENT FUNDING SUPPORT

This invention was made with government support under grant nos. GM-125976 and R21-GM129561, awarded by the National Institutes of Health. The United States government has certain rights in the invention.

### BACKGROUND

### 1. Field of the Invention

The field of this invention is medical testing and analysis. In particular, the invention pertains to probes, contact lenses, and methods for detecting analytes in tears, especially electrolytes (ions) such as sodium, chloride, and hydrogen ions.

### 2. Background of the Invention

For some conditions, such as keratitis, diabetes, certain autoimmune or inflammatory diseases affecting the eye, allergic eye disease, decreased tear production, and the like, and side effects from certain drugs, it is useful to detect the presence or concentration of analytes, such as electrolytes, biomolecules, or glucose, in tear fluid. However, at the present time there are limited practical ways to measure individual tear analytes (especially in basal tears) because the irritation caused by sample collection disturbs the analyte concentrations in tear fluid. Current in situ methods to measure these analytes, including contact lenses with an electronic sensor, come with instability, inefficiency, complexity, expense, or some combination thereof. In addition, currently used methods measure total ion or electrolyte concentrations, and not concentrations of each ionic species. Furthermore, probes available for these methods have been unsuccessful in detecting the analytes in physiological conditions and levels in tears, unlike other biological fluids such as blood or urine.

Modern medical practice depends on diagnostic testing of body fluids such as blood, serum, urine, and others. Measurements of electrolytes and proteins in such body fluids are routinely used to determine the overall health of patients. An array of specialized tests measure other target analytes such as, cholesterol, lipoproteins, hormones and cancer biomarkers. These tests are informative in samples such as blood because an aliquot (a representative sample) is readily obtainable without perturbation of the concentrations of the analyte of interest.

Measurement of electrolyte concentrations in tears is likewise a valuable tool in ophthalmology for research and diagnosis of dry eye disease (DED) and other ocular pathologies, as well as conditions such as total body dehydration. Electrolyte concentrations also can be altered by eye infections, keratitis, and other optical pathologies. However, unadulterated and representative basal tear samples are difficult to collect because the total volume is small and the chemical composition of the tears changes rapidly after physical contact or other perturbations of the eye. In addition, after collection, measurement of analytes, particularly ions, in tears is difficult because the highly sensitive immune and/or amplified assays for proteins and other biomarkers cannot be used to detect ion concentrations. While the electronics and optical technologies are available for fluorescence measurements of various ions in solution or in cells, these methods cannot be used in tears because the ion binding constants are not suitable for tears and the parent non-sensitive fluorophore (ISF) do not bind to lenses or contain chemically active groups. Currently, therefore, in contrast to many medical specialties, far fewer tests are available in ophthalmology and many diagnoses are based solely on visual examinations of the eye.

Tear fluid is produced by at least three processes. Basal level tears keep the cornea continually wet, lubricate the surface and clean out dirt and other particles. Reflex tears are produced when the eyes are irritated by foreign particles, touching, or vapors and irritants. Psychic tears are produced during crying and are induced by emotional stress or physical pain. These three types of tears have different concentrations of electrolytes, proteins and lipids. It is particularly difficult to obtain samples of basal tears because contact with the eye to collect a sample results in rapid production of additional tear fluid from the other processes, diluting the basal tears with other fluid. The normal volume of tears in each eye generally is about 6-7 μL, so it has been difficult to measure the individual ion concentrations in such a small sample without using a complex, expensive method like isotope dilution mass spectrometry.

Presently, clinical measurements of the individual ion concentrations in tears are non-existent for practical purposes, but the electrolyte concentrations of basal tear fluid are thought to be diagnostic of various medical conditions. For example, inflammation of the cornea is known to be associated with neutrophils which release protons when activated, suggesting that a pH decrease indicates an infection. Potassium has been reported to play a role in UV protection; calcium plays a role in mucin packaging and increased calcium concentrations have been linked to cystic fibrosis and age-related macular degeneration; magnesium deficiency has been linked to the incidence of glaucoma. It is known that dry-eye disease (DED) is associated with an overall increase in tear electrolyte concentrations.

Presently, tear sample testing for DED is limited to measuring the rate of tear production and the total electrolyte concentration. Until recently, the Schirmer test was the most widely used test to diagnose DED. This test uses strips of filter paper placed near the conjunctival sac of both eyes, left in place for 5 minutes. The distance of wetting of the filter paper correlates with the rate of tear production. A shorter wetting distance suggests the presence of aqueous deficient dry eye (ADDE). This test is not readily standardized, and there is no accepted wetting distance for diagnosis. Additionally, DED is a multi-factorial disease that can be due to other causes (e.g., Sjogren's syndrome, an autoimmune reaction affecting the moisture secreting glands of the eyes and mouth), or combinations of causes. The total electrolyte concentration in tears also can change with a change in lipid composition, for example due to dysfunction of the Meibomian gland, resulting in an excessive rate of water evaporation from tears, causing an increased electrolyte concentration. This condition, called evaporative dry eye (EDE), may not cause a decreased wetting distance in a Schirmer test and is difficult to diagnose without a detailed analysis of the lipid composition of tears, so this test has limited usefulness.

The Schirmer test is being replaced by in-office instrumental measurements of the total electrolyte concentration, which is reported as the total tear milli-osmolarity (TmOsm). A presumed unperturbed tear sample of basal tear fluid is obtained by momentary contact with a testing device that measures the electrical conductivity of the sample and calculates the total milli-osmolarity. TmOsm is presently regarded as the most reliable diagnostic test for DED, but even with this device, three repeated measurements on each eye are required for a clinically reliable result. Still, TmOsm does not provide the concentrations of individual ions in tears, which limits its usefulness to reveal DED in an individual patient, or the diagnosis of other conditions. The pH is not reported because of the low concentration of hydrogen ion (near 10⁻⁷M) and its insignificant contribution to the bulk conductivity. Additionally, the measurement is a singular data point in time, and cannot provide a time history of the ion concentration.

Despite the promises and potential convenience of measurements on basal tears, very few tests are available. At present, the method used to measure tear electrolytes is based on the electrical conductivity of small samples collected by momentary contact of a detector device with the eye. However, this approach provides a one-time reading and no information on the individual ion concentrations which contribute to the TmOsm. Recently, a microfluidic system has been reported for measurements of Na⁺, K⁺, Ca⁺⁺, and H⁺ (pH). This also is a one-time measurement and is not presently approved for clinical use.

Rapid and non-invasive measurements of hydration status is a medically important test for all age groups from infants to the elderly. Total body hydration is carefully controlled in humans within a narrow range. Mild levels of dehydration can have a significant impact on athletic performance and impair cognitive performance. In the elderly, dehydration was shown to play a role in dry eye disease. DED affects 10% to 30% of the population and results in a significant decrease in the quality-of-life of affected individuals. Diagnosis and treatment of DED is complicated because of the many factors which contribute to its severity, including dehydration, Sjogren's syndrome, Meibomian gland dysfunction and diabetes.

The effects of DED are not trivial and have a significant effect on the quality of life, workplace productivity and social interactions. For example, DED can make it difficult for individuals to read printed material or electronic displays. Because of the limited information from measurements of total electrolyte concentrations, it has been difficult to design useful drugs. There are only two FDA-approved drugs to treat DED. Restasis is an emulsion of cyclosporine, an immunomodulator, which decreases the inflammatory response in the conjunctival epithelial cells and increases tear production. Restasis is expected to be effective for patients with ADDE. A second drug is Xiidra which is thought to decrease release of cytokines.

DED and dehydration diagnoses could be improved by non-invasive measurements of the total electrolyte concentration in tears. There is widespread agreement that the TmOsm is the most reliable indicator for DED. The most reliable indicator for dehydration is thought to be the blood plasma osmolality (BPosm), however, determination of BPosm requires collection of a fresh blood sample and analysis, which is not convenient for DED detection during a doctor's office visit and not practical during athletic events, for example. TmOsm has been shown to correlate with BPosm, and TmOsm is regarded as the most promising marker for rapid measurements of an individual's hydration status.

For the reasons discussed above, there is a need in the art for new compounds, compositions, materials, systems, and methods for use in providing a way to detect and accurately measure concentrations of individual ions, including immediate results and continual measurements of tear electrolytes.

### SUMMARY OF THE INVENTION

Therefore, embodiments of the invention provides a fluorescent probe compound comprising at least one fluorophore that is sensitive to an electrolyte analyte selected from the group consisting of sodium, potassium, chloride, calcium, magnesium, and hydrogen, and that contains a hydrophilic region and a hydrophobic moiety, wherein the excitation wavelength of the fluorophore is from about 280 nm to about 750 nm; and wherein the hydrophobic moiety is configured to allow the fluorescent probe to bind non-covalently to a silicone hydrogel material. The hydrophilic region can be native to the fluorophore or fluorophore can be modified to contain a hydrophilic region.

In some embodiments, the fluorescent probe compound further comprises one or more linkers or spacers.

In some embodiments, the analyte is selected from the group consisting of sodium ion, chloride ion, potassium, hydrogen ion, calcium ion, magnesium ion.

In some embodiments, the fluorophore is selected from the group consisting of sodium green, SBFI, PBFI, CD 222, Fura-2, Indo-1, calcium green, and magnesium orange.

In some embodiments, the hydrophobic moiety is selected from the group consisting of an alkyl chain having 12 or more carbon atoms and an optional terminal amine group, poly-L-lysine with a molecular weight of about 70 kDa to about 150 kDa, lyso phosphatidyl ethanolamine, —NH₂—(CH₂)ₙ—CH₃ where n is 12-25, (—CH₂)ₙ—CH═CH₂ where n is 12-25, a saturated or unsaturated fatty acid chain having about 12-25 carbon atoms, phytyl groups, lysophospholipid, cholesterol, and mixtures thereof.

The invention also relates to, in certain embodiments, a silicone hydrogel contact lens comprising at least one fluorescent probe compound as described, bound to the silicone hydrogel contact lens either covalently or non-covalently. Preferably, the at least one fluorescent probe compound binds to the silicone hydrogel at the water-silicone interface and/or nonpolar areas.

In some preferred embodiments, the at least one fluorescent probe compound is not removed from the contact lens by exposure to tears for at least 1 day or at least 7 days.

In some embodiments, the silicone hydrogel contact lens contains a plurality of different fluorescent probe compounds are bound to the contact lens, which are bound throughout the material of the contact lens or are bound to different discrete areas of the contact lens.

In some embodiments, the silicone hydrogel contact lens comprises comfilcon A or stenfilcon A.

Some embodiments of the invention relate to a system comprising the silicon hydrogel contact lens of claim 8 and a wavelength ratiometric sensor.

Other embodiments of the invention relate to a method of measuring electrolytes in basal tears in a subject in need without perturbation of the tear composition, comprising: (a) placing the contact lens of claim 8 on the eye of the subject; (b) waiting at least about 10 minutes; (c) exposing the contact lens to light at the excitation wavelength of the at least one fluorescent probe; (d) detecting the emitted light from the contact lens; and (e) recording the wavelength-radiometric measurements or intensity decays of the emitted light.

### BRIEF SUMMARY OF THE DRAWINGS

FIG. 1A and FIG. 1B are photographs of certain embodiments of contact lenses in use, including single analyte lenses (FIG. 1A) and a multi-analyte lens (FIG. 1B).

FIG. 2 is an illustration of one type of SiHG material showing the positions of probes, as indicated.

FIG. 3 provides structures of selected fluorophores for use with the invention.

FIG. 4 provides structures of selected calcium and magnesium fluorophore probe compounds with indicates hydrophobic moieties.

FIG. 5 provide selected structures of calcium and magnesium sensitive fluorophores.

FIG. 6 shows chemical structures of example sodium and potassium fluorophore probes with example hydrophobic moieties.

FIG. 7 shows Chemical Scheme 1, synthesis information for selected sodium fluorophore probes as an example, with description of certain main features of the invention.

FIG. 8A and FIG. 8B show the semi-interpenetrating polymer networks of contact lenses. The dots show the locations of the ionic species (analytes) in the lenses. In the figures, water channels and silicone channels are shown in two lens materials (FIG. 8A: Comfilcon A, Biofinity™ and FIG. 8: Stenfilcon A, MyDay™). See arrows. Dots show the locations of the ionic species in the lenses.

FIG. 9 is a block diagram and flow chart that illustrates example fluorescent light properties that can be measured, according to various embodiments. The three methods on the right provide measurements independent of total intensity. The three methods on the right provide measurements independent of total intensity.

FIG. 10 is a block diagram that illustrates an example measurement system, according to an experimental embodiment.

FIG. 11 is a block diagram that illustrates a computer system upon which an embodiment of the invention can be implemented.

FIG. 12A, FIG. 12B and FIG. 12C are photographs of SG-PL in a Biofinity™ contact lens under room light (FIG. 12A) and with diffuse 473 nm wavelength illumination for 0 mM NaCl (FIG. 12B) and 150 nM NaCl (FIG. 12C).

FIG. 13 presents anisotropy decays of SG in buffer and the three sodium probes in Biofinity™ contact lenses, as indicated.

FIG. 14A is a set of confocal intensity images of SG-C16 in Biofinity™ contact lenses for 0 mM NaCl and 140 mM NaCl. FIG. 14B is a set of lifetime images of SG-C16 in Biofinity™ contact lenses for 0 mM NaCl and 140 mM NaCl. Biofinity™ contact lenses for 0 mM NaCl and 140 mM NaCl. Images at 0 mM NaCl were acquired at two different focal planes.

FIG. 15A and FIG. 15B each are sets of photographs showing confocal intensity and lifetime images of SG-LPE in Biofinity™ contact lenses. Images at 0 mM NaCl (FIG. 15A) were acquired at two different focal planes. Images at 100 and 240 mM NaCl were acquired at the same focal plane.

FIG. 15A and FIG. 15B each are sets of photographs showing confocal intensity and lifetime images of SG-PL in Biofinity™ contact lenses with 0 and 100 mM NaCl. Images at 0 mM NaCl were acquired at two different focal planes.

FIG. 17A shows sodium-dependent emission spectra of SG-C16 in Biofinity™ contact lenses. FIG. 17B shows intensity decays of SG-C 16 in Biofinity™ contact lenses.

FIG. 18A and FIG. 18B are sodium-dependent emission spectra (FIG. 18A) and intensity decays (FIG. 18B) of SG-LPE in Biofinity™ contact lenses.

FIG. 19A and FIG. 19B are sodium-dependent emission spectra (FIG. 19A) and intensity decays (FIG. 19B) of SG-PL in Biofinity™ contact lenses.

FIG. 20A and FIG. 20B are sodium-dependent emission spectra (FIG. 20A) and intensity decays (FIG. 20B) of Sodium Green (SG) in 20 mM MOPS buffer with 8 mM KCl.

FIG. 21A and FIG. 21B present sodium-dependent intensities and lifetimes, respectively for SG in MOPS buffer and the three sodium probes as indicated in Biofinity™ contact lenses. Lifetime measurements were performed on Fluotime 300 instrument. Numerical values correspond to mid-points of sodium-dependent responses.

FIG. 22A and FIG. 22B show the reversibility of a SG-C16-labeled Biofinity™ contact lens measured by intensity (FIG. 22A) and lifetime (FIG. 22B) with repeated cycling between no sodium and 220 mM NaCl. Measurements were performed on the center area of the lens using FLIM instrumentation.

FIG. 23A and FIG. 23B show the reversibility of a SG-LPE-labeled Biofinity™ contact lens measured by intensity (FIG. 23A) and lifetime (FIG. 23B) with repeated cycling between no sodium and 220 mM NaCl. Measurements were performed on the center of the lens using FLIM instrumentation.

FIG. 24A and FIG. 24B show the reversibility of a SG-PL-labeled Biofinity™ contact lens measured by intensity (FIG. 24A) and lifetime (FIG. 24B) with repeated cycling between no sodium and 220 mM NaCl. Measurements were performed on the center area of the lens using FLIM instrumentation.

FIG. 25A presents data on sodium-dependent intensity and FIG. 25B on lifetime responses of SG-PL in Biofinity™ lenses in the absence and presence of the HSA, mucin or lysozyme, as indicated. Measurements were performed on the center area of the lens using FLIM instrumentation.

FIG. 26A and FIG. 26B show sodium-dependent emission spectra (FIG. 26A) and intensity decays (FIG. 26B) of SG-PL in Biofinity™ contact lenses.

FIG. 27A and FIG. 27B show sodium-dependent emission spectra (FIG. 27A) and intensity decays (FIG. 27B) of SG-PL in MyDay™ contact lenses.

FIG. 28A and FIG. 28B present binding curves (log scale) of sodium binding to SG-PL in Biofinity™ and MyDay™ lenses as measured by intensities (FIG. 28A) or lifetimes (FIG. 28B). Numerical values indicate the mid-points of the sodium response.

FIG. 29A and FIG. 29B show the reversibility of sodium binding to SG-PL in MyDay™ lens (FIG. 29A: intensity; FIG. 29B: amplitude weighted lifetime).

FIG. 30A (normalized intensity) and FIG. 30B (amplitude weighted lifetime) show sodium-responses of SG3-PL labeled MyDay™ lens in the absence (MOPS buffer only) and presence of 1 mg/ml of the HSA, Mucin and Lysozyme as indicated. Numerical values indicate the midpoints of the curves.

FIG. 31A and FIG. 31B are emission spectra (FIG. 31A) and intensity decays (FIG. 31B) of SPQ-C18 in Biofinity™ lenses, with increasing chloride concentration, pH 7.2 phosphate buffer.

FIG. 32A and FIG. 32B are emission spectra (FIG. 32A) and intensity decays (FIG. 32B) of SPQ-C18 in MyDay™ lenses, with increasing chloride concentration, 20 mM MOPS pH 7.3 buffer.

FIG. 33 shows the reversibility of chloride quenching of SPQ-C18 in Biofinity™ lenses measured by intensity (FIG. 33A) and lifetime (FIG. 33B).

FIG. 34. Reversibility of chloride quenching of SPQ-C18 in MyDay™ lenses measured by intensity (FIG. 34A) and lifetime (FIG. 34B).

FIG. 35A and FIG. 35B are Stern-Volmer plots for chloride quenching of SPQ-C18 in Biofinity™ and MyDay™ contact lenses, as indicated.

FIG. 36A and FIG. 36B show excitation and emission spectra, respectively, of 6HQ-C18 in Comfilcon A lens. FIG. 36C shows the pH-dependent excitation ratio for 6HQ-C3 in buffer and 6HQ-C18 within a Comfilcon A SiHG lens. Emission was monitored at 580 nm and λex=350 nm.

FIG. 37 shows a comparison of lifetime Stern-Volmer plots for SPQ-C3 in water and SPQ-C18 in a Stenfilcon A (Aspire™) contact lens.

FIG. 38 presents binding affinity curves of SG-PL with sodium in Biofinity™ and MyDay™ lenses as measured by intensities at peak maximum (FIG. 38A) or amplitude-weighted lifetimes (FIG. 38B). Numerical values indicate the mid-points of the sodium response.

FIG. 39 shows sodium-dependent emission spectra (FIG. 39A) and intensity decays (FIG. 39B) of SG-PL in Biofinity™ contact lenses.

FIG. 40A, FIG. 40B, FIG. 40C, and FIG. 40D are photographs showing SG3 spotted on a discrete area of Biofinity™ lenses in the sodium concentrations indicated. FIG. 40E provides an intensity line tracing along the dotted lines shown in FIGS. 40A-40D.

FIG. 41 shows the absorption and emission spectra of SPQ-C18 (OD-MQB) and SG-PL as indicated. The absorption spectrum of SPQ-C18 is in MeOH and that of SG-PL in 20 mM MOPS buffer pH 7.3. Emission spectra are in Biofinity™ lenses.

FIG. 42A and FIG. 42B show the chloride (FIG. 42A) and sodium (FIG. 42B) responses of a MyDay™ contact lens labeled with both SG-PL and SPQ-C18.

FIG. 43A, FIG. 43B, and FIG. 43C show sodium and chloride responses of a MyDay™ contact lens labeled with both SG-PL and SPQ-C18. FIG. 43A: intensity decays of SPQ-C18; FIG. 43B and FIG. 443C: emission spectra at two excitation wavelengths as indicated.

FIG. 44A, FIG. 44B, and FIG. 44C show data for 1,8-ANS in 1-hexanol, ethanol and two types of SiHG lenses as indicated. FIG. 44A: normalized fluorescence intensity; FIG. 44B: counts; FIG. 44C: fractional intensities.

FIG. 45 shows results for 1,8-ANS from tests in water-methanol mixtures, acetonitrile and MyDay™ lens. ANS in solvents are with the same concentration. The excitation wavelength is 375 nm. FIG. 45A: intensity; FIG. 45B: intensity (counts).

FIG. 46 shows results for Prodan™ in water, acetonitrile and MyDay™ lens. The Prodan™ concentrations in water and acetonitrile are the same. The excitation wavelength is 375 nm.

FIG. 47A is a photograph showing a rabbit in the restrainer and wearing a contact lens. FIG. 47B and FIG. 47C provide emission spectra and intensity decays, respectively, of SG-poly-lysine (SG-PL) in a Biofinity™ lens on a rabbit eye with without and with 150 mM Na⁺.

### DETAILED DESCRIPTION

### 1. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

As used herein, the term “about” means plus or minus 20 percent of the recited value, so that, for example, “about 0.125” means 0.125±0.025, and “about 1.0” means 1.0±0.2.

As used herein, the term “dry-eye disease (DED)” refers to a condition associated with an overall increase in tear electrolyte concentrations. The term “aqueous deficient dry eye (ADDE)” refers to DED resulting from decreased aqueous tear production.

As used herein, the term “basal tears” refers to the tears present in the eye in the absence of any physical or chemical irritation.

As used herein, the term “modified or derivatized” in the context of a molecule or compound indicates that the native compound has been changed, generally by adding a moiety or group the compound, optionally covalently.

As used herein, the term “fluorophore” refers to a fluorescent chemical compound that can re-emit light upon excitation. Fluorophores include non-reactive fluorophores (FL), fluorophores with reactive groups (R-FL) that can react or be activated to react with other molecules, fluorophores linked to ionic side chains that bind to lenses (I-FL), fluorophores linked to hydrophobic groups (H-FL), or any fluorophore known in the art. Fluorophores generally described herein refer to ion-sensitive fluorophores (ISF) unless stated to the contrary.

As used herein, the terms “fluorescent probe compound,” “ion-sensitive fluorophore (ISF) probe,” “fluorophore probe,” and similar language refers to a chemical compound comprising an optionally modified fluorophore with an analyte binding region or portion, a hydrophilic region or portion, and a hydrophobic region or portion. This can include any fluorophore which displays different spectral properties in response to ions. The spectral changes can be due to ion binding where a ion-fluorophore complex is formed, or a fluorophore which is quenched by diffusive or collision contact with a quencher. Fluorophores are typically available commercially in non-reactive form, and cannot be bound to lenses. Binding of the probes to lenses is accomplished by modifying the fluorophore by covalent attachment of the fluorophore to polar, ionic and/or hydrophobic groups, forming a probe.

As used herein, the term “fluorophore probe” according to embodiments of the invention, refers to a fluorophore that non-covalently binds to a SiHG contact lenses as described herein or, optionally, is covalently attached to the contact lens.

As used herein, the term “analyte binding” or “analyte binding portion” refers to a molecule or region of a molecule that specifically binds to or contacts without binding to an analyte of interest.

As used herein, the term “hydrophilic portion” of a molecule refers to a “water-loving” region of the structure that is attracted to water or other polar molecules.

As used herein, the term “hydrophobic portion,” or region, area, or moiety of a molecule refers to a region that is repelled by water or polar molecules and attracted to fatty compounds.

As used herein, the term “electrolyte” includes, but is not limited to Na⁺, K⁺, Ca⁺⁺+Mg⁺, H⁺, and Cl⁻, or any ion which is found in tears.

As used herein, the term “water-silicone interface” refers to the regions of a lens where the local chemical composition changes from mostly polar water (or tear fluid) to non-polar silicone.

### 2. Overview

A device and method is provided related to fluorescent contact lenses for determining electrolyte ion concentrations, such as sodium, chloride, and hydrogen ion concentrations, in the physiological range for tears. Fluorophores known to be sensitive to ions such as Na⁺ and Cl⁻ were modified to provide spontaneous non-covalent binding to silicone hydrogel (SiHG) contact lenses. Two examples of lenses are demonstrated using commercially available Biofinity™ (Comfilcon A) or MyDay™ (Stenfilcon A) lenses. Both lenses are widely prescribed, the Biofinity™ lenses for 30-day use, and the MyDay™ lenses for one-day use. Biofinity™ lenses have a high silicone content and MyDay™ lenses a lower silicone content. For both lenses, the responses to Na⁺ and Cl⁻ were found to be completely reversible and not sensitive to proteins in tears, including lysozyme, serum albumin and mucin type 2.

Still other aspects, features, and advantages are readily apparent from the following detailed description, simply by illustrating a number of particular embodiments and implementations. Other embodiments are also capable of other and different features and advantages, and its several details can be modified in various obvious respects, without departing from the spirit and scope of the invention. Accordingly, the drawings and description are illustrative in nature, and not restrictive.

### 3. Summary of Results

Results demonstrate that ion-sensitive fluorophores can be derivatized in several ways for binding to silicone hydrogel contact lenses in a manner that unexpectedly allows them to be used in a contact lens for detection of the analytes at physiological concentrations. The fluorophore lenses for sodium sensing showed useful spectral changes in response to sodium. The responses were reversible and were not affected by several proteins present in tears. The starting material Sodium Green (SG) has a high affinity for sodium, and would be saturated or non-responsive in tears. For this reason, an individual skilled in the art would not have selected SG to measure sodium in tears. However, it is reported here that sodium binding is much weaker when the fluorophore is modified and bound to lenses, in the tear physiologic sodium concentration range. In a similar way, an individual skilled in the art would not select SPQ, described below, for the uses in the present invention because it will be too strongly quenched at tear chloride concentrations. However, it is reported here that reduced chloride quenching of SPQ when bound to lenses, for a useful spectral change in the physiological chloride concentration in tears.

Thus, contact lenses were developed that respond to the electrolytes in tears. The rapid evolution of consumer electronics makes it possible to design and fabricate an EL-CL reader for intensity ratios or lifetimes from labeled lenses worn by patients. For example, a device can be located at an eye examination station in the ophthalmologist's office. Such a device could be a small desktop-type steady-state fluorometer for wavelength-ratiometric measurements and/or a time-resolved instrument for intensity decays. All the electronics for time-correlated single photon counting can be placed on a single computer circuit board. Such contact lenses can be used to measure additional tear electrolytes, such as for research and diagnosis of DED and other ocular conditions. Clinical research projects can be initiated to correlate individual ion concentrations with specific ocular diseases. Smaller EL-CL readers can be developed as well, for example a portable hand-held device based on a cell phone camera. Portable reading devices are made practical by the low power consumption of CIAs which is 100-fold less than CCD devices.

### 4. Embodiments of the Invention

A. Introduction and General Discussion

The tear film forms a complex three-layer structure which consists of a hydrophobic layer adjacent to the corneal cells, a central aqueous region containing electrolytes and proteins, and a hydrophobic outer layer which slows water evaporation. The lower hydrophilic layer is highly permeable to ions, and the ion concentrations are carefully controlled in vivo to maintain the corneal cells. When a contact lens is placed on the eye, it sits in the central aqueous layer, directly exposed to the tear fluid. The fluid volume in a single eye is about 7 μl, and the eyes respond rapidly upon any contact resulting in increased secretion of the lacrimal glands and changes in ion concentrations. The turnover for basal tears in the eye is about 2.0-2.2 μL/min, allowing rapid equilibration of the tear fluid in the eye and in the lens.

Specific fluorescent probes are bound non-covalently or covalently to hydrogel (preferably silicone hydrogel) contact lenses so that aqueous samples of tears containing analytes can flow past the fixed probes on the subject's eye. The fluorophore probes are modified to contain a hydrophobic portion so that the probes are attracted to the silicone interstices. There can be a hydrophilic portion to maintain contact with the aqueous sample solution. In some embodiments, a functional probe contains an analyte-binding portion to contact or capture an analyte from the sample solution, and a fluorophore portion that will change at least one measurable property of its fluorescent emissions when an analyte binds to or contacts the analyte-binding portion. Fluorescence spectral changes can also occur by diffusive contact with quenchers. Thus, when the contact lens is immersed in an aqueous fluid (tears), the probes are exposed to the analytes in the aqueous solution and can bind or detect an analyte. Several probes are described here, as well as the lens materials and methods for detecting certain analytes. The invention preferably relates to electrolytes such as metal ions like sodium, potassium, calcium, magnesium, zinc, mercury, lead, and H⁺ or pH, and the like, or chloride, so any analyte binding portion that binds to an electrolyte of tears can be used with the invention. The probe in some embodiments should have a binding affinity which allows detection of the analyte at the physiological concentrations found in tears.

These techniques discussed here enable the practitioner to determine the presence and concentration of analytes in tear fluid of a subject's eye using a low-cost commercial contact lens as a substrate and readily available imaging technology as a remote monitoring sub-system. The concentrations of analytes in tears can be measured on a moving eye, in vivo, by lifetime-based or wavelength-ratiometric sensing, which are independent of total emission intensity. These labeled contact lenses provide a new tool for research in ophthalmology, for diagnosis of diseases or damage to the cornea. Unexpectedly, these modified probes are able to measure the analytes at physiological concentrations in a practical manner, bound to a contact lens. Although the methods discussed here are designed to measure tears in the eye of a subject, the methods also can be used in other contexts to measure analytes in a fluid.

The methods herein advantageously avoid collecting basal tears from a subject, which is difficult and can cause eye irritation. Unlike some previous methods, there is no need for complex embedded electronics, which increases cost so that the invention allows this analysis to become a daily use or portable product, preferred for reasons of safety and patient choice (compliance). The suitability of this approach for detection of analytes at physiological concentrations is clear.

Attempts to use fluorophores that respond to analyte concentrations within commercial contact lenses have been hindered by the unavailability of suitable probes that can detect an analyte, for example sodium or chloride, at physiological concentrations in tears. Most probes of this type were designed for operation in body fluids such as blood, which have different ranges of analyte concentrations. This invention uses modified probes that surprisingly have a suitable binding affinity for this purpose.

A composition, material, method, apparatus and system are described for a silicone hydrogel based assay and contact lens. In the following description, numerous specific details are set forth to provide a thorough understanding of the invention. It will be apparent, however, to one skilled in the art, that the present invention can be practiced without these specific details. Some well-known structures and devices are shown in block diagram form. Some embodiments of the invention are described below in the context of contact lenses made of a material that includes a silicone hydrogel substrate and fluorescent probes attracted to a water-silicone interface of the substrate, for which fluorescence is measured remotely.

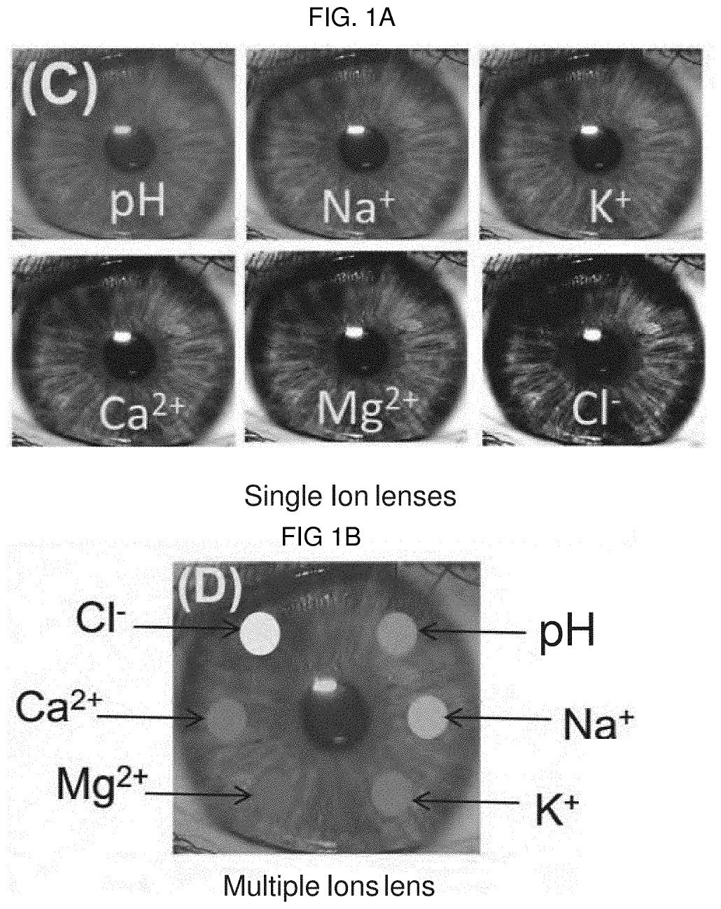

Referring to FIG. 1A, a contact lens can be configured to measure one or more electrolyte in tears without contacting the eye and without altering tear composition. Such an electrolyte contact lens can be used, for example, in a doctor's office after insertion of the lens and a short waiting period of about 10 minutes or longer for the tears to equilibrate and regain the normal electrolyte concentrations. The lens can be worn only for the time needed to take the measurements, for an hour or two hours, or longer. Alternatively, the lens can be a prescribed lens to be worn continually by the patient and read with a hand-held reading device at advantageous intervals to provide a longer term readout of data over time.

Such an electronic device is possible due to the many advances in electronics, detectors, and iris tracking technologies. Generally, the limiting factor has been ion-specific fluorophores that bind to contact lenses and respond to tear electrolytes at physiologically relevant concentrations. The literature describes ion-sensitive fluorophores (ISF) that are sensitive to electrolytes such as pH, Na⁺, K⁺, Ca⁺⁺Mg⁺⁺ and Cl⁻. However, these ISF are designed to be water-soluble and responsive to intracellular concentrations of ions or blood/serum concentrations. As a result of water-solubility, the probes are washed out of the lenses rapidly because tear fluid is replaced approximately every 5 minutes. The probes also may not have the spectral response at physiological concentrations in tears which make them useful for the purpose.

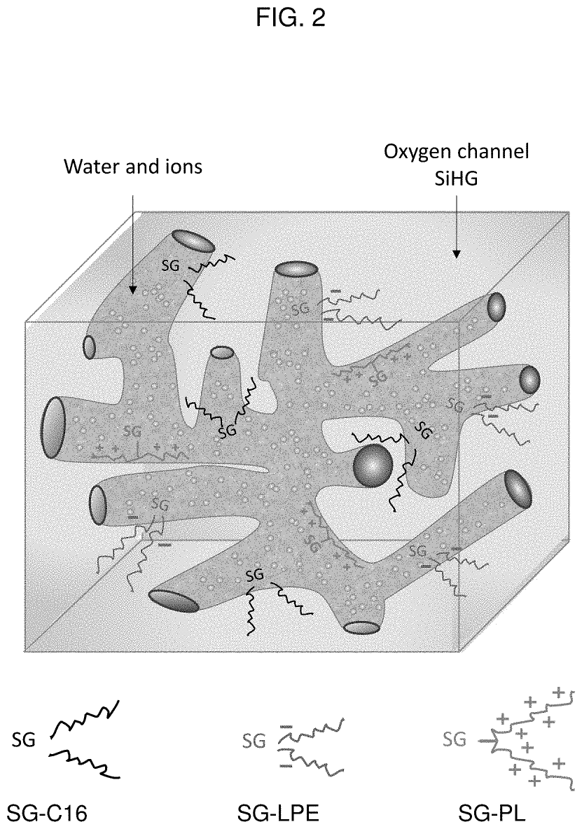

The polymer chemistry for contact lenses allows for the design of ion-sensitive fluorophores that bind to the lenses. Typical soft contact lenses are based on silicone hydrogels (SiHG). Typical lenses contain regions that are non-polar silicone-rich regions and polar regions that are all water or tear fluid (FIG. 2). Other silicone hydrogels can contain less silicone and more water. The high water content and high rates of water (or tear) transport in and through these lenses is evidence that the lenses contain continuous water channels across the entire lens (a semi-interpenetrating polymer network (IPN) as shown in FIG. 2. This structure suggests the existence of a polar to non-polar interface regions comparable to a cell membrane-water interface.

The interface regions of the IPN provide a location to bind ion-specific fluorophores that remain in the aqueous channels in contact with the electrolytes. These ion-specific fluorophores contain non-polar groups that bind to the silicone-rich regions. This approach can be used for ion-sensitive fluorophore compounds with hydrophobic side chain modifications that bind to the silicone-rich regions of the lenses. Thus, the invention provides a contact lens sensitive to tear electrolytes such as sodium by using, for example, the sodium-sensitive fluorophore, Sodium Green, with the attachment of a hydrophobic side chain.

Some lenses contain only a small amount of silicone. To accommodate this, a sodium-sensitive fluorophore linked to poly-L-lysine (PL) can be synthesized for binding at the interface or electrostatic binding to the negative charges in SiHG or non-silicone hydrogel (HG) lenses. These concepts are validated by the synthesis and testing of three sodium-sensitive fluorophores that bind to the SiHG Comfilcon A Biofinity™ lenses and result in a sodium-sensitive contact lens (FIG. 1B).

B. Probes for Use in the Invention

Probes according to the invention comprise at least one fluorescent portion, an analyte binding portion which binds specifically to or is collides with an analyte, a hydrophilic portion, a hydrophobic portion, and optionally also contains one or more linkers or spacers to assist in avoiding possible steric hindrance. Probes of the invention are described and shown by structure throughout this specification.

A suitable ion-sensitive fluorophore probe for this invention preferably has ion binding affinities within the physiological range of electrolytes in tears. Additionally, the fluorophore should bind tightly to the contact lens without washout from tear replacement for hours or days of use. The binding is typically provided to the fluorophore by a ligand which has high affinity for the lenses such that it can bind at physiological concentrations. A preferred silicone hydrogel contact lens binds the probe compound such that it is not removed from the contact lens by exposure to tears for at least 1 day, 2 days, 3 days, 1 week (7 days), 2 weeks, 3 weeks, 30 days or longer.

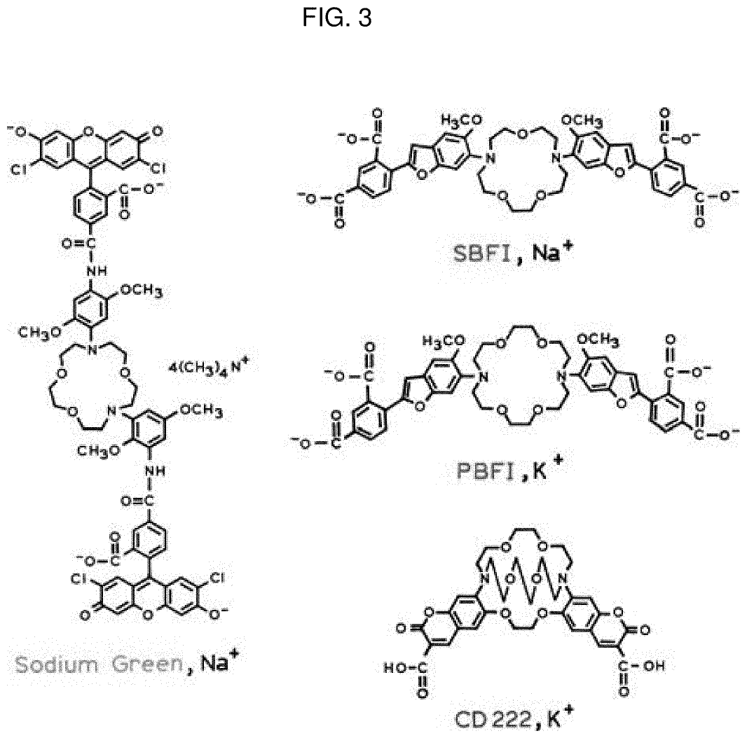

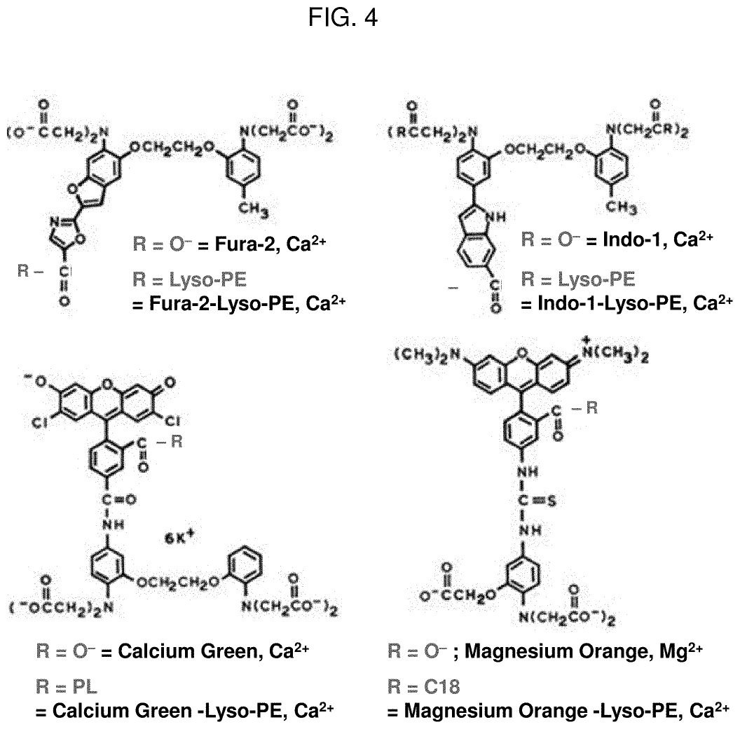

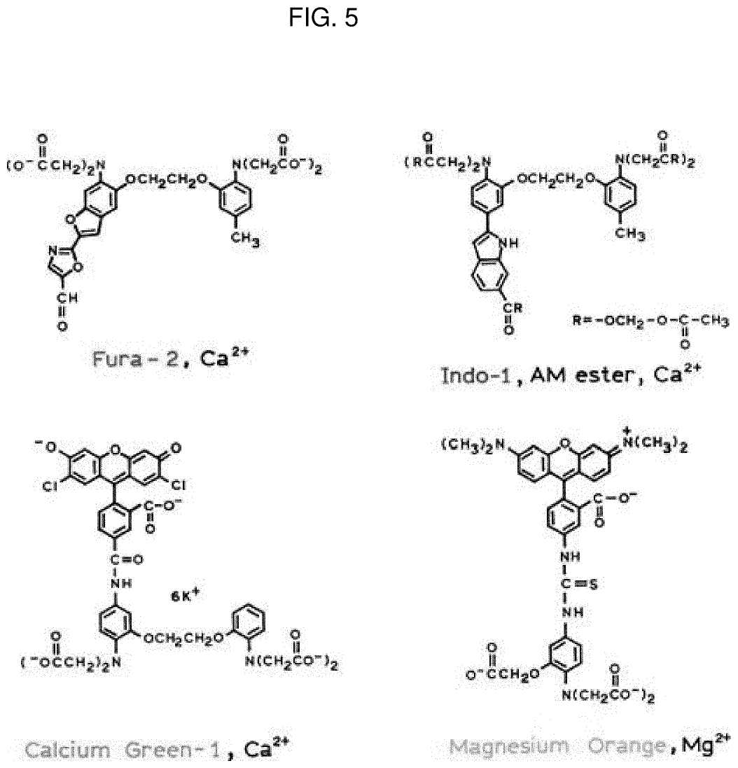

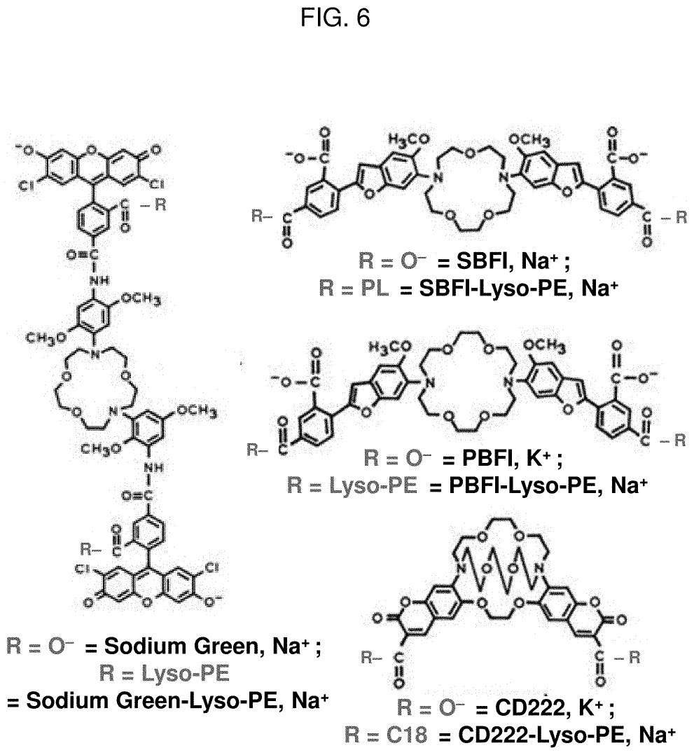

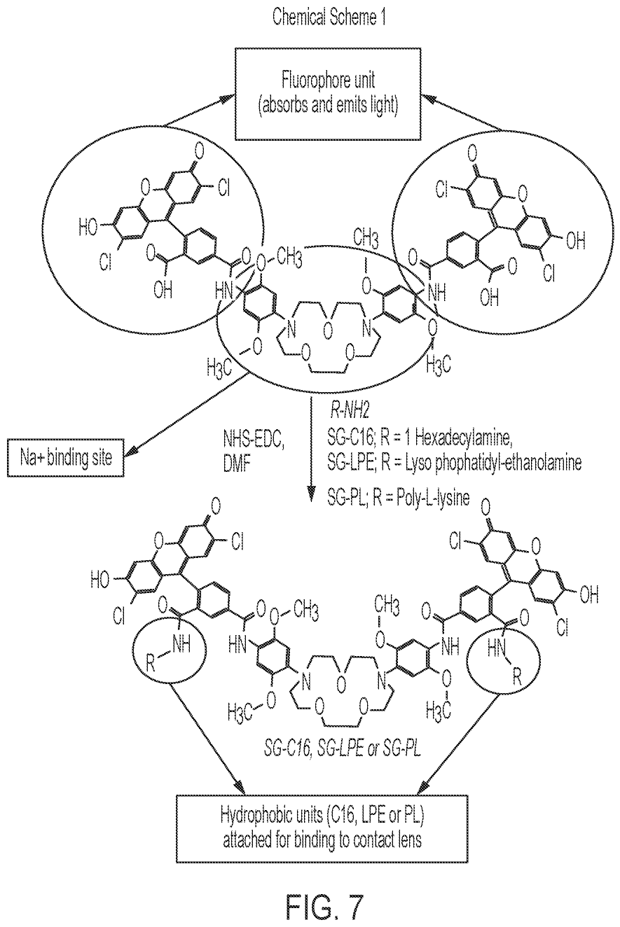

A suitable fluorophore for use in the probe generally is useful with excitation wavelengths in the visible to NIR range (i.e., about 280 nm to about 900 nm, preferably about 300 nm to about 800 nm, about 300 nm to about 700 nm, or about 350 nm to about 700 nm, be photostable, and be non-toxic in the amounts which can be released from the lens to the eye. In addition, the fluorophore needs to either display a change in lifetime upon changes in ion concentrations, or contain a non-ion sensitive fluorophore for wavelength-ratiometric measurements. Preferred fluorophores include, but are not limited to hydrophobic derivatized those described in FIG. 3, FIG. 4, FIG. 5, FIG. 6, and FIG. 7, such as Sodium Green or Sodium Green derivatives, CoroNa™ Green, CoroNa™ Red, SBFI, PBFI, PBFT, CD 222, BODIPY-azacrown compounds, Fura-2, Indo-1, calcium green, magnesium orange, SNAFL compounds and derivatives, SNARF compounds and derivatives, fluorescein and derivatives, BCECF, Mag-Quin1, Mag-Quin 2, Mag Fura 1, Mag Fura 2, Mag Green, MagOrange, and the like, including probes known in the art to be ion sensitive. Other multi-ring structures with hydroxy or amine groups such as naphthol compounds can be used.

See Table 1, below for concentrations of useful electrolytes. The concentrations can be higher or lower in certain disease states.

TABLE 1
Electrolyte Concentrations in Normal Tears.

| Ion | Concentration | |
|---|---|---|
| pH | 6.5-7.6 | |
| H⁺ | 25-316 | nM |
| Na⁺ | 132 | mM |
| K⁺ | 24 | mM |
| Ca⁺⁺ | 0.8 | mM |
| Mg⁺⁺ | .06 | mM |
| Cl⁻ | 118-138 | mM |

The hydrophobic portion of the fluorophore probe in most cases is a hydrophobic moiety added to the probe by covalent attachment to the fluorophore. The probe is derivatized to add this hydrophobic group, which is discussed herein.

A preferred hydrophilic moiety is poly-L-lysine (PL) with molecular weight of about 70 kDa to about 150 kD (approximately 500 to 1000 lysine units), but the poly-L-lysine size can vary, for example about 50 kDa, or about 70 kDa, or about 100 kDa, or about 120 kDa, and the like, or a range of molecular weights as available commercially. Other polyelectrolytes can also be used. If additional hydrophilic areas are to be attached, hydrophilic groups such as poly-arginine of about the same sizes, or groups or chains with oxygens, nitrogens or hydrophilic groups can be used. PL is a positively charged biopolymer that tends to bind negatively charged surfaces like contact lens and glass. Other positively charged biomolecules, like lysozyme, positively charged polyelectrolytes such as polyallylamine, and other similar polymers can be used as hydrophilic groups, can be used as can be determined by those of skill in the art.

The local environment of a contact lens can affect the dissociation constant of the fluorophore probe for proton or metallic ligands. Thus, the binding affinities of ion-sensitive fluorophores can change. A binding affinity measured for a fluorophore in buffer compared to the same fluorophore measured while bound to and in the unique environment of a particular contact lens may be quite different. The change in affinity can be different for different probes and in different contact lenses, therefore, a single fluorophore may not be useful in all lenses, and the modifications to the fluorophore can change these effects.

Uses of the invention involve, for example, using the fluorescent probe-bound contact lens to determine individual ion concentrations in tears using a remote detection device in a doctor's office or remotely (e.g., at home, in emergency situation, at an athletic event, and the like). In one embodiment, the contact lens includes at least one probe, such as those described herein, or others that may be known in the art, modified so that the analyte binding affinity is in the correct range.

In some embodiments, the probes can be used as lifetime-based sensors, while in other embodiments the probes can be used as wavelength-ratiometric sensors with reference fluorophores. Thus, according to at least one embodiment, a device and method is provided to bind sodium-sensitive fluorophores with other ion-sensitive fluorophores and/or contact lens polymers, resulting in an electrolyte contact lens with higher accuracy, ease-of-use, and lower costs compared to typical devices. The fluorophores bind to contact lenses and are not removed by washing for days with buffers or wearing on the eye of a subject for hours, days, or weeks. The intensity and lifetime changes of the probes are generally reversible to ion analyte concentration changes and not affected by several proteins in tears including lysozyme, human serum albumin and mucin type II.

In summary, probes according to the invention generally are modified with hydrophobic groups that promote very strong non-covalent binding to the contact lens material and may have activated side chains such as succinimidyl esters, maleimide, S-acetylmercapto or NHS esters, and the like. The probes have appropriate binding affinities for the analytes. Alternatively, the probes can be covalently linked to the lens by any method known in the art.

Probes for pH Measurements

A number of pH-sensitive fluorophores are known in the art and some are commercially available. Two possible fluorophores for pH measurements are a fluorescein derivative BCECF (2′,7′-bis(2-carboxyethyl)-5(6)-carboxyfluorescein) and a rhodamine derivative SNARF-1. The probes have pKa values close to the range of pH values found in tears (7.4 for BCECF and 7.5 for SNARF-1). BCECF has been studied extensively for sensing in tissue cultures. Both probes can be excited in the same wavelength range from 460 nm to 530 nm, but the emission spectra are widely separated. BCECF can be selectively observed at 525 nm and SNARF-1 at 650 nm. BCECF can be used as a wavelength ratiometric probe using different excitation wavelengths to provide measurements of the pH. The intensity of BCECF increases about 8-fold from low to high pH and the lifetime changes from 3.0 to 3.9. This lifetime change is suitable for lifetime-based sensing (LBS). SNARF-1 has the same advantages as BCBEF. The intensity changes about 5-fold and the mean lifetime at 650 increases with pH from 0.94 ns at low pH to 1.45 at high pH. SNARF-6 has a chemical structure very similar to SNARF-1, and displays a larger lifetime change from 0.94 to 4.51 ns from low to high pH. The emission spectrum shifts with pH SNARF-1 can also provide wavelength-ratiometric measurements of the pH.

Using the pH indicators SNARF-1 or SNARF-6, for example, allows one to adjust the ion-sensitive range to the physiological range needed for detecting the analyte in tears. This change is possible by selection of the observed emission wavelength. Because both forms of the SNARF probes (low and high pH forms) are fluorescent, the intensity decays can be multi-exponential at intermediate pH values. The observed fractional emission from each form results in a different intensity decay at each wavelength. As a result, the apparent pKa can be selected by changing the observed emission wavelengths. The intensity decay displays two decay times 4.9 ns and 0.95 ns, assigned to the low and high pH form, respectively. The relative contribution to the intensity decay depends on the observation wavelength. The apparent pKa can be changed from near 6.0 at 640 nm to 8.0 at 580 nm. This change occurs because the lifetime is an intensity-weighted value. The actual pKa does not change. The ability to choose the chosen apparent pKa can be used to keep the probe response in the optimal physiological range. The BCECF and SNARF dyes are derivatives of fluorescein and rhodamine and are not expected to be toxic to eyes. The structure requires attachment of a ligand for binding to the lenses, and in the case of BCECF, removal of the acetoxymethyl (AM) groups. The use of SNARF-1 will require adding a ligand to one of the free carboxyl groups.

Potential hydrophobic side chains for producing spontaneous binding at interfaces in SiHG lenses can include side chains that contain separating units of polyethylene glycol or arginine peptide. Below are example probe structures for of SNARF-1 bound to lyso-PE and carboxy SNARF-6.

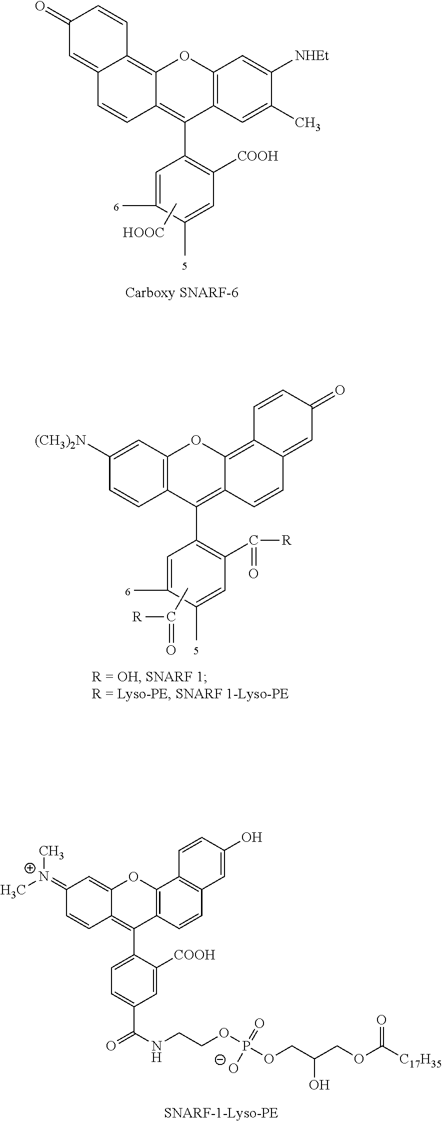

Other example potential R groups that can act as hydrophobic moieties include

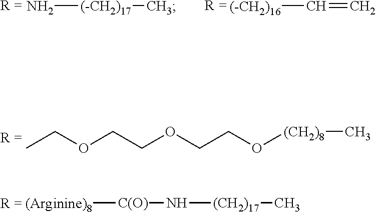

Resonance energy transfer (RET) offers some advantages for fluorescence sensing and can be used in the invention. Changes in energy transfer can occur due to changes in analyte proximity or due to analyte-dependent changes in the absorption spectrum of the acceptor. RET-based sensing simplifies the design of a fluorophore. For collisional quenching or analyte recognition probes, the probe should be specifically sensitive to the analyte. When RET is used, there is no need to find a single molecule that has both the needed sensitivity and the right fluorescence spectral properties since the donor and the acceptor can be separate molecules. The donor should be selected for use with the light source to be used and the acceptor chosen to display a change in absorption in response to the analyte. Alternatively, an affinity sensor can be based on a changing concentration of acceptor around the donor due to the association reaction. This idea can be used to create lifetime-based sensors for pH, pCO₂, and glucose. See Table 2, below for a non-inclusive list of pH probes.

TABLE 2
Spectral and Lifetime Properties of pH Probes.

| | Excitation | Emission | | Lifetime (ns)^{a} | |
|---|---|---|---|---|---|
| Probe^{b} | λ_{B}(λ_{A}) [nm] | λ_{B}(λ_{A}) [nm] | Q_{B} (Q_{A}) | τ_{B} (τ_{A}) | pK_{A} |
| BCECF | 503 (484) | 528 (514) | ~0.7 | 4.49 (3.17) | 7.0 |
| SNAFL-1 | 539 (510) | 616 (542) | 0.093 (0.33) | 1.19 (3.74) | 7.7 |
| C. SNAFL-1 | 540 (508) | 623 (543) | 0.075 (0.32) | 1.11 (3.67) | 7.8 |
| C. SNAFL-2 | 547 (514) | 623 (545) | 0.054 (0.43) | 0.94 (4.60) | 7.7 |
| C. SNARF-1 | 576 (549) | 638 (585) | 0.091 | 1.51 (0.52) | 7.5 |
| | | | (0.047) | | |
| C. SNARF-2 | 579 (552) | 633 (583) | 0.110 | 1.55 (0.33) | 7.7 |
| | | | (0.022) | | |
| C. SNARF-6 | 557 (524) | 635 (559) | 0.053 (0.42) | 1.03 (4.51) | 7.6 |
| C. SNARF-X | 575 (570) | 630 (600) | 0.160 (0.07) | 2.59 (1.79) | 7.9 |
| Resorufin | 571 (484) | 528 (514) | NA^{c} | 2.92 (0.45) | ~5.7 |
| HPTS | 454 (403) | 511 | NA | NA | 7.3 |
| [Ru(deabpy)(bpy)₂]²⁺ | 450 (452) | 615 (650) | NA | 380 (235) | 7.5 |
| Oregon-Green | 489 (506) | 526 | 0.65 (0.22) | 4.37 (2.47) | 1.8 |
| DM-Nerf | 497 (510) | 527 (536) | 0.88 (0.37) | 3.98 (2.50) | 1.6 |
| Cl-Nerf | 504 (514) | 540 | 0.78 (0.19) | 4.00 (1.71) | 2.3 |

τ_{B} and τ_{A} refer to the mean lifetimes of the acid and base forms, respectively.
NA indicates not available.
bpy is 2,2′-bipyridine.
deabpy is 4,4′-diethylaminomethyl-2,2′-bipyridine.
HPTS is 8-hydroxypyrene-1,3,6-trisulfonate.

Preferred pH probes are based on fluorescein:

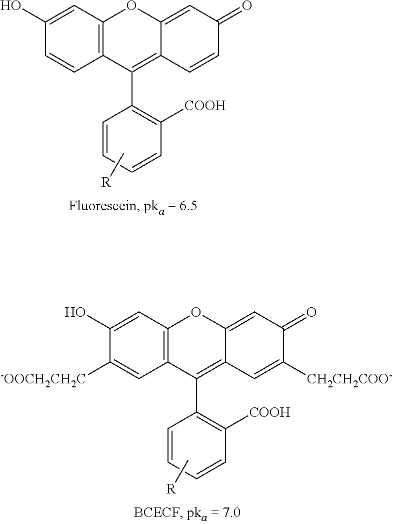

where R is —H for fluorescein and R is —COOH and placed at the 5- or 6-position for carboxyfluorescein.

Sodium-Sensitive Probes

Measurements of Na⁺ or K⁺ concentration by fluorescence are more difficult than pH. In the case of pH sensing, a weak covalent bond is broken when dissociation of a proton occurs. This dissociation can change the hybridization of electron orbitals in the fluorophore, and changes to its absorption and emission spectra. Only ionic bonds and non-covalent bonds are formed by Na⁺ and K⁺, however. As a result, the fluorophore needs to respond to the near field around the ion, typically by ion binding to a chelator which in turn affects the fluorophore.

Many sodium-sensitive fluorophores (SSF) are known. The requirements for use in a contact lens are the ability to distinguish the free and sodium-bound fluorophores from the spectral data and to have sodium affinity constants in the physiological range for tears. Almost all SSF are based on azacrown ethers which are attached to the fluorophores by the one or two azacrown nitrogen atoms.

A surprising consequence of the studies reported here is that the binding constants of the probes were found to change dramatically when bound to a contact lens. SG itself in buffer solution displays a Na⁺ binding constant near 10 mM, which suggested that SG would be fully saturated in tear fluid with 132 mM Na⁺ and therefore not useful for detecting physiological levels of the analyte. However, addition of the linker, the hydrophobic moiety, and/or binding to the lenses decreased the SG binding constant to near 100 mM so that it became useful in tear fluid analysis.

This application describes an example Na⁺-sensitive contact lens. Sodium Green was covalently linked to polylysine (PL) or a C16 chain, which rapidly bound to both Biofinity™ lenses and more slowly bound to MyDay™ lenses. Binding of Na⁺ to the lenses resulted in an approximate 3-fold increase in intensity and lifetime. The SG emission spectrum does not shift upon Na⁺ binding, but the lifetimes can be used to measure the Na⁺ concentration.

Both CoroNa™ Green and CoroNa™ Red (sodium ion indicators) can be excited with visible wavelengths near 450 nm. The emission spectra are widely separated with peaks at 520 and 580 nm, and the probes can be selectively observed at these wavelengths. The binding constants are reported to be 82 mM for CoroNa™ Green and 200 mM for CoroNa™ Red, which are ideal for sodium sensing in tears. CoroNa™ Green can be used for LBS because multi-exponential decay in the absence of Na⁺ is about 2-fold faster than the mono-exponential decay when bound to Na⁺. Lifetime data was not found for CoroNa™ Red, but from the intensity increase when bound to Na⁺ it is likely that the lifetime also increases with Na⁺ binding. These probes therefore are considered useful for Na⁺ measurements.

Chloride-Sensitive Probes

Heavy atoms like bromine and iodine can act as collisional quenchers. Chloride is a less effective quencher and does not quench all fluorophores, but is still important in biological systems because of its prevalence in biological fluids. The quenching constant thus depends on the probe chemical structure. Representative chloride probes include SPQ (6-methoxy-N-(3-sulfopropyl)quinolinium), SPA (N-sulfopropyl-acridium), MQAE (1-(ethoxycarbonylmethyl)-6-methoxyquinolinium bromide), MACA (N-methylacridium-9-carboxamide), MAMC (N-methylacridium-9-methylcarboxylate), and Lucigenin (N,N,N-dimethyl-9,9′-bisacridium nitrate). See Lakowicz, Principles of Fluorescence Spectroscopy, chapter 19 for more discussion. This reference is incorporated by reference herein. See also the structures below in Table 3. The probes shown are not modified to promote binding to a contact lens, but can be modified as discussed herein.

TABLE 3
Structures of Representative Unmodified Chloride Fluorophores.

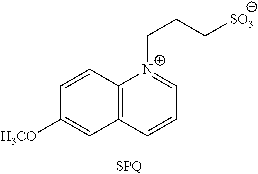

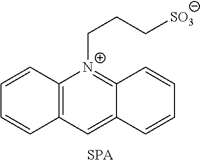

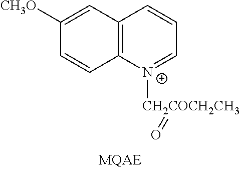

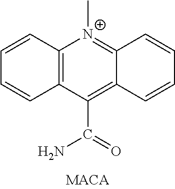 [SMILES: C[n+]1c2ccccc2c(C(N)=O)c2ccccc21]

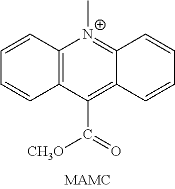

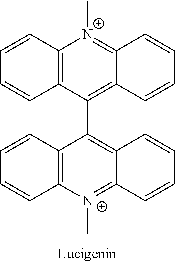 [SMILES: C[n+]1c2ccccc2c(-c2c3ccccc3[n+](C)c3ccccc23)c2ccccc21]

The selection of fluorophores sensitive to chloride is different from other ions because chloride sensing does not involve binding to the fluorophore. Two different chloride probes useful for the invention are shown below. The OD-MQB probe has two rings, and MAMC has three rings, all electron deficient. These probes can be selectively observed at 440 nm for OD-MQB and at 550 nm for MAMC.

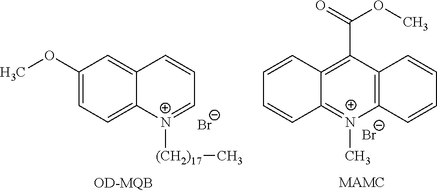

Both of these probes can be excited with incident light near 400 nm. In the excited state the probes are quenched (returned to the ground state without emission) upon contact with chloride. The probes are not destroyed by quenching but remain available for further excitation and quenching. The intensities (I) and lifetimes (t) are analyzed using the Stern-Volmer equation F₀/F=1+K[Cl⁻] or τ₀/τ=1+K[Cl⁻], where F₀ and τ_{D}/τ are the intensities and lifetimes in the absence of chloride, and K is the quenching constant and the inverse of the [Cl⁻] needed for 50% quenching. For collisional quenching, the values of F₀/F and τ₀/τ are typically the same. A possible difficulty with the chloride probes is that the emission spectra do not shift on quenching. This difficulty of detecting chloride can be avoided by using lifetime-based sensing, or by using a reference fluorophore and the wavelength ratiometric method. One example of a probe suitable for this method is an SPQ analog covalently linked to a fluorophore which is not sensitive to Cl⁻ quenching. The fluorophore not sensitive to chloride has an emission maximum at 50 mm which can be readily measured separately from the SPQ-like emission at 450 mm.

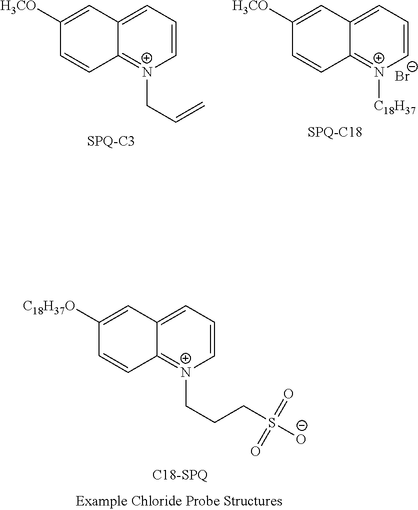

Osmolarity Sensing

There has been a rapid introduction of new optical methods for in-vivo studies of tear films and corneal health. Some of these methods are an automation of measurements which are already measured, such as tear break-up times (TBUT), using optical imaging and computer analysis in place of visual observation by an ophthalmologist or automated imaging of the Meibomian gland. Other examples include the use of interferometry and measurements of both the tear films and cornea using optical coherence tomography. Raman spectroscopy has been proposed to detect adenovirus in tears. These methods provide images but are not sensitive to or specific for electrolytes in tears. When these methods were first introduced, there was no body of evidence to correlate the measurements with disease states but such evidence is being rapidly accumulated.

The situation is different for an EL-CL. Specific ocular changes occur with changes in specific ion concentrations in tears, such as correlating magnesium concentrations in tears with glaucoma for example. However, conductivity measurements cannot detect changes in individual ion concentrations. For these reasons, the EL-CL can be rapidly adopted for research in ophthalmology and for use during routine eye exams.

Other Probes

See Table 4 for additional probes suitable for use with the invention and FIG. 3 for sodium and potassium probe structures. Chemical modification of these fluorophores is performed for binding to contact lenses.

TABLE 4
Spectral and Lifetime Properties of Mg⁺, Na⁺, and K⁺ Probes.

[symbol]

| | Excitation | Emission | | Lifetime (ns)^{c} | |
|---|---|---|---|---|---|
| Probe [symbol] | λ [symbol] (λ [symbol] ) [nm] | λ [symbol] (λ [symbol] ) [nm] | Q [symbol] (Q [symbol] ) | [symbol] | K [symbol] (mM) |
| Mg [symbol] ⁺ Probe [symbol] | | | | | |
| Mag-Quin-1 | 348 (335) | 499 (490) | 0.015 (0.009) | 0.57 (10.3) | 6.7 |
| Mag-Quin-2 | 353 (337) | 487 (493) | 0.003 (0.07) | 0.84 (7.16) | 0.8 |
| Mag- [symbol] -2 | 369 (330) | 511 (491) | 0.24 (0.30) | 1.64 (1.72) | 1.0 |
| Mag- [symbol] -5 | 369 (332) | 505 (482) | NA^{c} | 2.52 (2.39) | 2.3 |
| Mag- [symbol] | 349 (330) | 480 (417) | 0.36 (0.59) | 1.71 (1.90) | 2.7 |
| Mag- [symbol] -Red | 453 (427) | 659 (681) | 6.012 (0.007) | 0.38 (0.35) | 2.5 |
| Mg Green | 506 | 532 | 0.64 (0.42) | 0.98 (3.63) | 1.0 |
| Mg Orange | 550 | 575 | 0.13 (0.34) | 1.06 (2.15) | 3.9 |
| Na⁺ Probes | | | | | |
| SBF [symbol] | 348 (335) | 499 (490) | 0.645 (0.087) | 0.27 (0.47) | 3.8 |
| SBFO | 354 (343) | 515 (500) | 0.14 (0.44) | 1.45 (2.09) | 31.0 |
| Na Green | 506 | 535 | 7-fold [symbol] | 3.34 (2.38) | 6.0 |
| K [symbol] Probes | | | | | |
| PBF [symbol] | 336 (338) | 557 (507) | 0.24 (0.72) | 0.47 (0.72) | 5.1 |
| CD 222 | 396 (363) | 480 (467) | 3.7-fold | 0.17 (0.71) | 0.9 |

[symbol]

F and B refer to the free and cation-bound forms of the probes, respectively. [symbol]
Abbreviations: SBF [symbol] sodium-binding benzofuran isophthalate, SBFO, sodium-binding benzofuran oxazole; PBFL potassium-binding benzofuran isoph [symbol] . [symbol]
NA: not available [symbol]
Q [symbol] /Q [symbol] [symbol]
indicates data missing or illegible when filed

From Lakowicz, J. R., Principles of Fluorescence Spectroscopy, 3^{rd} edition, 2006. Springer Science+Business Media, LLC. Chapter 19, pages 623-641.

Calcium probes also are available based on the BAPTA chelator which binds Ca⁺⁺ with affinities near 100 nM. See Table 5 and the structure of Cal-520, below, showing an NHS ester and linker for binding to lenses. See also FIG. 4 and FIG. 5 for structures. Chemical modification is needed for binding to contact lenses.

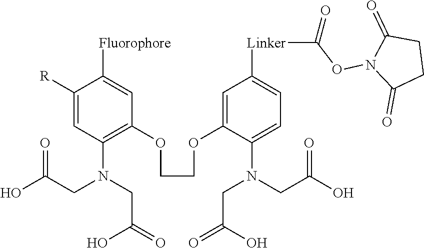

### Cal-520-C16: R=(CH2)15CH3

### Cal-520-PEG: R=(O—CH2-CH2)nOH

### Cal-520: R=H

TABLE 5
Spectral and Lifetime Properties of Ca⁺⁺ Probes.

[symbol]

| | Excitation | Emission | | Liftime (ns) [symbol] | |
|---|---|---|---|---|---|
| Probe | λ [symbol] (λ [symbol] ) [nm] | λ [symbol] (λ [symbol] ) [nm] | Q [symbol] (Q [symbol] ) | [symbol] | K [symbol] (nM) |
| Quin-2 | 356 (336) | 500 (503) | 0.03 (0.14) | 1.35 (11.6) | 60.0 |
| F [symbol] -2 | 362 (335) | 518 (510) | 0.23 (0.49) | 1.09 (1.68) | 145.0 |
| Indo-1 | 349 (331) | 482 (398) | 0.38 (0.50) | 1.40 (1.66) | 230.0 |
| Fura Red | 472 (436) | 657 (637) | Low QY^{b} | 0.12 (0.11) | 140.0 |
| BTC [symbol] | 464 (401) | 531 | NA [symbol] | 0.71 (1.38) | |
| Pluo-3 | 504 | 526 | 4 [symbol] -fold | 0.04 (1.28) | 390 |
| Rhod-2 | 550 | 581 | 100-fold | NA | 570 |
| Ca Green | 506 | 534 | 0.06 (0.75) | 0.92 (3.60) | 190 |
| Ca Orange | 555 | 576 | 0.11 (0.33) | 1.20 (2.31) | 185 |
| Ca Crimson | 588 | 611 | 0.18 (0.53) | 2.55 (4.11) | 185 |
| Ca Green-2 | 505 | 536 | ~100-fold [symbol] | NA | 550 |
| Ca Green-5N | 506 | 536 | ~30-fold | NA | 14,000 |
| Ca Orange-5N | 549 | 582 | ~5-fold | NA | 20,000 |
| Oregon Green | | | | | |
| BAPTA-1 | 494 | 523 | ~14-fold | 0.73 (4.0) | 170 |
| BAPTA-2 | 494 | 523 | 35-fold | NA | 580 |
| BAPTA-5N | 494 | 521 | NA | NA | 20,000 |

[symbol]

F and B refer to the Ca [symbol] free and Ca [symbol] bound forms of the probes, respectively. [symbol]
Low quantum yield. [symbol]
BTC, commarin benzothiazole-based indicator. [symbol]
NA: not available. [symbol]
The term [symbol] -fold refers to the relative increase in fluorescence upon cation binding. [symbol]
indicates data missing or illegible when filed

From Lakowicz, J. R., Principles of Fluorescence Spectroscopy, 3^{rd} edition, 2006. Springer Science+Business Media, LLC. Chapter 19, pages 623-641.

Magnesium probes also are available, including Mag-INDO-1, based on the calcium probe INDO-1. See also FIG. 4 and FIG. 5. Probes for metals such as zinc, mercury, and lead also can be useful and are contemplated for use with the invention.

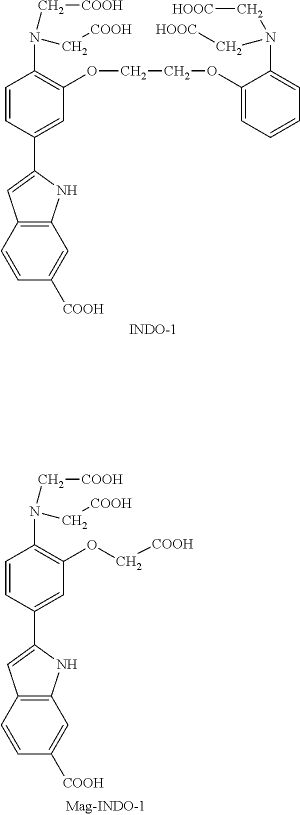

See also certain chelating groups specific for the indicated cations, which can be useful as probes:

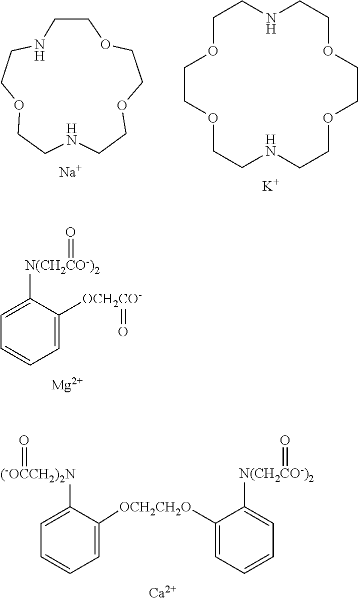

Probe Modifications

The probes described herein generally will be modified by addition of hydrophobic moieties that allow the probe to strongly bind (non-covalently or covalently) to the contact lens material. Appropriate moieties for this use include fatty acid chains and the like, with 12 or more carbon atoms, and preferably are selected from fatty acid chains having about 12 to about 25 carbon atoms, phytyl groups, lysophospholipid, cholesterol or steroid derivatives, hydrophobic peptides such as polylysine, unsaturated fatty acids, lysophosphatidyl ethanolamine (lyso-PE), and the like, or mixtures thereof. Most preferred moieties are Group 2 fatty acid chains with 12 or more carbon atoms, or alkyl chains with a terminal amino group. The choice of which hydrophobic group or groups should be added to the fluorophore probe can be determined by the person of skill based on the disclosures herein and knowledge in the art concerning fluorescent probe molecules. The hydrophobic moiety preferably is covalently attached to the probe fluorophore molecule, optionally using a linker.

For example, the uses of hydrophobic groups for sodium probes have been discussed above as examples. See also FIG. 6 for example structures. This figure shows the chemical structures of a visible wavelength Na⁺ probe Sodium Green (left) and UV-analogues Na⁺ probe (SBFI). PBFI and CD222 are selective K⁺ probes. R groups show example modifications to add a hydrophobic moiety to the compounds and probe analyte specificity, but others, such as poly-L-lysine (MS about 70 to 150 kD or about 500 to 1000 lysine units), or an alkyl chain of 12 or more carbon chains, and the like, also can be used. Probes for a particular ion can be selected by the skilled artisan. For example, by modifying a fluorophore, it becomes useful for contact lens applications. In this particular example, SG is the fluorophore. A hydrophilic moiety remains in aqueous regions of contact lens and binds with sodium ions in tears. The hydrophobic moieties (e.g., C-16, LPE and PL) tend to bind (non-covalently) to the hydrophobic silicone regions of a contact lens. See also FIG. 7 for synthesis information and a general description of some main features of the invention. The same hydrophobic units with other probes listed, such as those in FIG. 3 and FIG. 5 can show response to respective ions within the contact lens environment.

See below for two examples of chloride fluorophore probes showing main aspects of the structure.

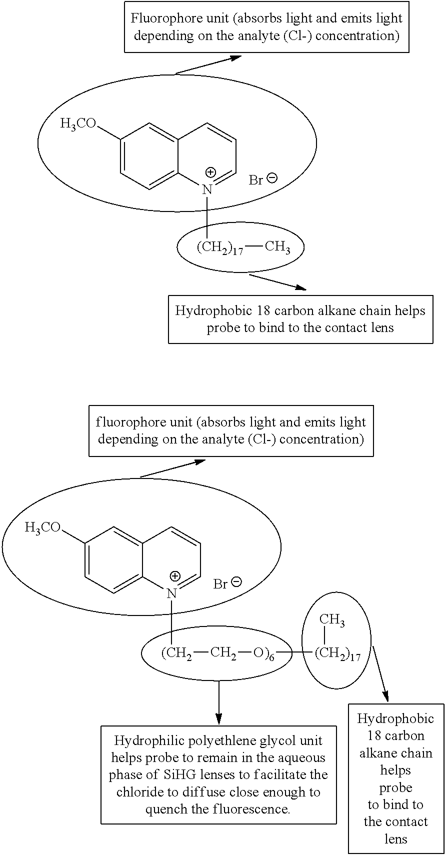

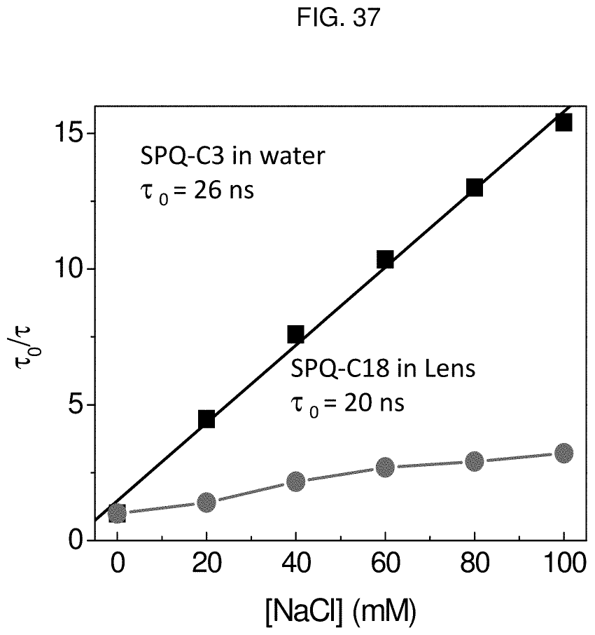

For these probe examples (diffusional quencher probes), there is no specific analyte binding site. in this probe. The same types of hydrophobic units, (e.g., C16 or C18, LPE or PL) can be used to bind these probes to the contact lens. The quenching constant for SPQ-C18 (in lens) is reduced by about 10-fold as compared to that of SPQ-C3 in buffer (FIG. 37). This unexpected result made SPQ-C18 useful for tear chloride sensing using contact lenses. By changing the overall probe structure, binding constants or quenching constants of the probes can be modulated. For some Na⁺ or other analyte binding probes, which are already hydrophilic in nature and remain in the aqueous regions of the lens, adding an additional hydrophilic moiety is not needed. Thus, the sodium probes, SG-C16, SG-LPE and SG-PL, for example, are not shown with added hydrophilic modifications.

### C. Lenses

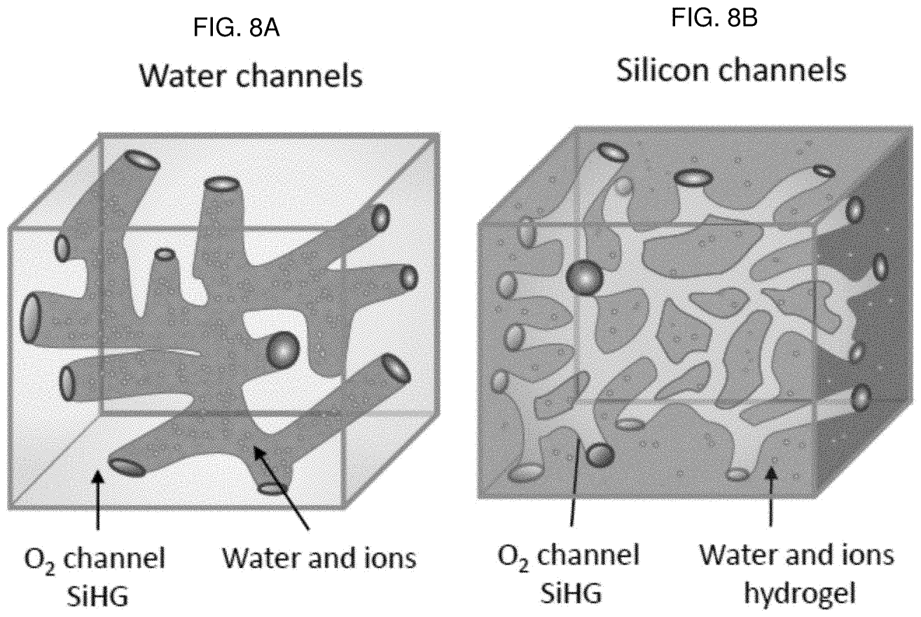

SiHG lenses have high permeability to both oxygen and ionic species. SiHG contact lenses are mostly silicon but contain a semi-interpenetrating polymer network (INP) which consists of continuous channels across the lens for tear or water transport. Oxygen transport occurs through the silicone regions (see FIG. 8A and FIG. 8B), as continuous channels for oxygen. In Biofinity™ lenses, there are continual pores from the front to the back of the lens which allows rapid transport of ions. MyDay™ lenses are claimed to have the opposite 3D structure, consisting of thin continuous silicon chains with the remaining value filled with a standard hydrogel and/or tear fluid. The MyDay™ lenses therefore have less hydrophobic properties of the Biofinity™ lenses or the high silicone content which can result in eye irritation. Both lenses are optically clear, which indicates the IPN network has sub-wavelength dimensions which do not scatter light. The pore size in the Biofinity™ lenses is thought to be about 50 nm in diameter. See FIG. 8.

In SiHG lenses, oxygen moves through the continuous silicone regions and is even greater in concentration there than an equivalent thickness of water. Because of the high oxygen transport, some SiHG lenses are approved for 30-day continuous wear including sleeping with the lenses in place. Presently, there are numerous types of contact lenses (CLs) on the market with slightly different polymers and surface treatments to reduce hydrophobicity. See Table 6, below.

TABLE 6
Selected Hydrogel and Silicone Hydrogel Contact Lenses.

| | | | Water | |
|---|---|---|---|---|
| Polymer | Trade Name | Manufacturer | (%) | Dk |
| Lotrafilcon A (SiHG) | Night and Day ™ | CIBA Vision | 24 | 140 |
| Galyfilcon A (SiHG) | Acuvue Advance ™ | Johnson & | 47 | 60 |
| | | Johnson | | |
| Comfilcon A (SiHG) | Biofinity ™ | Cooper Vision | 48 | 128 |
| Stenfilcon A (SiHG) | MyDay ™ | Cooper Vision | 54 | 80 |
| Latrofilcon B (SiHG) | Air OptixAqua ™ | Ciba Vision/ | 33 | 138 |
| | | Alcon | | |
| Nelfilcon A (HG) | Aqua Release ™ | Ciba Vision | 69 | 26 |
| Nelfilcon A (HG) | Dailies ™ | Ciba Vision | 31 | 26 |

Dk indicates the measure of oxygen permeability through the contact lens with a certain pressure difference, in a given time [Dk = 10⁻¹¹ (cm³ O₂ cm)/(ml sec Barrer)].

A second generation SiHG lens, Biofinity™, is made with the SiHG polymer Comfilcon A which contains a high silicon content (near 40%); MyDay™ lenses are made with Stenfilcon A (see FIG. 8A and FIG. 8B). The MyDay™ lenses have a very low silicone content, reported to be 4.4%, while maintaining high oxygen transmission. The remaining space in the MyDay™ lenses is probably a standard HG, but these details are not released by the company. In practice, the Biofinity™ and MyDay™ lenses are essentially the same in terms of comfort and absence of complications.

### D. Probe Binding (Labeling) to Contact Lenses

Non-covalent binding of the probes to the contact lens is performed by incubation of the lens in a solution of probes. The probes are prepared and then diluted in water at a concentration of about 0.3 μM to about 5 μM, preferably about 0.5 μM to about 2 μM, and most preferably about 1 μM, at room temperature. For a lens designed for a single electrolyte or with even distribution of one or more probes throughout the lens (i.e., uniformly labeled lenses), the lens is prepared by washing the lens to remove any contaminants, incubating the lens in the solution of probes (a single probe or more than one probe) for about 1 hour or longer (for example about 2 hours, about 4 hours, about 8 hours, overnight, 1 day, or longer as necessary). Biofinity™ lenses require less incubation time while MyDay™ lenses require several hours. The length of incubation can easily be determined by the skilled practitioner as a matter of routine. After incubation with the probes, the lenses are extensively washed with deionized water to eliminate any loosely bound probe from the lenses before being used. Unless stated otherwise, all in vitro work was performed in 20 mM MOPS buffer, 8 mM KCl, pH 7.2, at room temperature, with variable concentrations of NaCl.

Research use or clinical applications of the inventive methods advantageously involve measurements of more than a single ionic species. Measurements can be limited to uniformly labeled contact lenses, but for measurement of multiple analytes, multiple probes can be bound to the entire contact lens using this method. A preferred method of detection using many different fluorophores is to place different probes in separate distinct areas of the lens. For lenses designed to detect multiple analytes, the probe solutions are applied to the lenses in discrete areas (see FIG. 1B) as described above by applying a solution of probes such that only a portion of the lens is allowed to wet, repeating with different probe solutions, and incubating to allow binding. The diluted probe solutions mentioned above were dropped on the lens where the small portion of the lens was allowed to wet. Subsequently a second probe can be labeled similarly on to the lens

Certain contact lenses, such as MyDay™ lenses and other modern lens materials have lower silicone content (MeDay™ has about 4.4% silicone content) and contain a hydrogel component of the poly-HEMA type. The hydrogel portion of these types of contact lenses can be hydrolyzed to generate free carboxylic acid groups on the lens surface, which can be used for probe tethering with a free amine containing probes. As an example, MyDay™ lenses were treated with NaOH solution and then washed rigorously with deionized water before being used for amide forming reaction with fluorescein cadaverine. An NHS-EDC activation procedure was used. Fluorescein cadaverine is a fluorescein derivative with pH sensing ability. See the structure below.

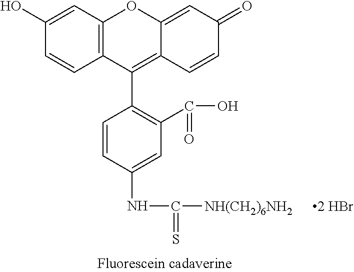

This kind of approach can be used to covalently graft other analyte sensitive dyes to the contact lens.

### E. Use of Contact Lenses

Patients

The invention disclosed herein is contemplated to be useful to subjects, including animal or human subjects. Laboratory animals, livestock, companion animals, and the like are contemplated as subjects, as well as humans. The contact lenses of the invention are contemplated to be useful as diagnostic tools for use in hospitals, doctor's offices (for example, ophthalmologists), at home, or in the field. Conditions such as dry eye disease, Sjogren's syndrome, allergic eye disease, rheumatoid arthritis, lupus, scleroderma, graft vs. host disease, sarcoidosis, thyroid disorders, vitamin A deficiency, total body dehydration, keratitis, and the like, can be diagnosed using the invention. Therefore, subjects preferably are suffering from or are suspected of suffering from these or related conditions. Dry eye disease or its symptoms can also be caused by certain medications (for example, antihistamines, decongestants, hormone replacement therapy, antidepressants, and drugs for high blood pressure, acne, birth control and Parkinson's disease) or procedures such as laser eye surgery or other procedures involving the eye. Subjects undergoing these treatments also can find the invention useful.

### F. Summary

Thus, the invention relates to a contact lens that is configured to allow non-contact measurements of the individual electrolytes in tears. These lenses are based on remote detection of fluorescence from ion-sensitive fluorophores that bind to contact lenses and are sensitive to specific ions. The contact lenses can be designed for detection of any desired set of analytes, depending on the patient's needs. One preferred embodiment of the device includes lenses that are sensitive to both sodium and chloride, which are the dominant electrolytes in tears and can provide a clinically useful estimate of the osmolality of tears (TmOsm; e.g., with calibration and testing). In particular embodiments, for example, sodium and chloride are the dominant electrolytes in blood, plasma and tears, so a sodium and chloride-sensitive contact lens (NaCl-lens) can be used for rapid non-invasive detection of dry eye disease or whole body hydration. The chloride-sensitive fluorophore possesses a hydrophobic side chain as described in the art. The sodium-sensitive fluorophore was linked to poly-L-lysine, which binds to SiHG lenses. The sodium and chloride sensitive lens (NaCl-lens) lenses were made using two very different contact lens polymers which demonstrates the wide applicability of our approach.

The concept of the invention is facilitated by the literature on fluorophores sensitive to a variety of cations and anions. Silicone hydrogels that contain low polarity regions rich in silicone and regions which are aqueous or tear fluid. The interface between these regions advantageously provides a location to bind ISF that contain hydrophobic side chains, as reported here. Unexpectedly, it was discovered here that modifying or derivatizing the fluorophore probes with an appropriate hydrophobic group not only permits binding to the lens material, but also affects the binding affinity of the analyte detecting portion of the probe. Modification of the binding affinity of the probes allows one to adjust the binding affinity to ensure the probe will detect the analyte at the necessary (physiological) concentrations, even though the probe may have a different non-useful binding affinity when in solution.

### F. Detection Methods and Computer Systems

Measurement of emitted fluorescent light preferably is performed under conditions where background emission from the fluorophore probes in the contact lens does not affect light detection due to specific activation/quenching of the fluorophore. In general, the main limit on sensitivity of fluorescence is background emission from the sample. Background emission is always present and usually strong. Surprisingly, the methods of this invention are effective under normal conditions in a doctor's office or laboratory, even though interfering background emission would have been anticipated. Here, background emission has not been a problem.

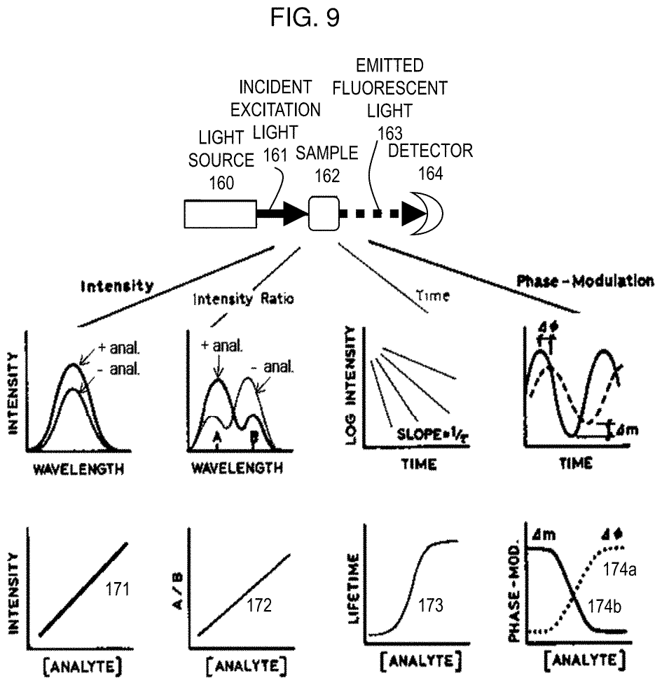

FIG. 9 illustrates example fluorescent light properties that can be measured according to various embodiments of the invention. In each embodiment, light 161 of a particular wavelength or wavelength band from a light source 160 is incident on a sample 162, such as material 100 with fluid 190 and analytes 192 therein. Fluorescent light 163 at a different wavelength or band is emitted in response and detected at an optical detector 164 that puts out a digital electrical signal or an analog electrical signal that can be digitized at an analog to digital converter (ADC). Although the emitted fluorescent light 163 is depicted in the same direction as the incident light for purposes of clarity of the diagram, the emitted fluorescent light 163 can be at a different angle than depicted. Example different properties of the emitted fluorescent light 163, among others known in the art, which can be measured include: intensity of the emitted light, represented by the column of graphs on the left; intensity ratio at two or more different wavelengths as indicated by the second column of graphs; intensity decay with time as indicated by the second column of graphs; and phase shift or modulation relative to the incident light The latter two properties both reflect the lifetime of the emitted fluorescent light 163 after the incident light is turned off, e.g., fluorescent lifetime after a pulse of incident light.

Referring now to the graphs of FIG. 9, if any of the measurable properties from a probe composition 150 are found to depend on the concentration of the analyte 192 in the fluid 190 for a range of analyte concentrations of interest, then that probe composition 150 is suitable for forming material 100. The top graph in each column shows examples of different responses for two different concentrations of an analyte, assuming for purposes of illustration that there is a useful dependence of that property on concentration of analyte. The bottom graph in each column depicts calibration curves for each property assuming for purposes of illustration that there is a useful dependence of that property over a useful range of concentrations of analyte.

Still referring to the graphs of FIG. 9, as examples, the top graph on the left column shows that fluorescent intensity forms a peak in a wavelength band at low concentrations of analyte 192 (labeled as “− anal” in the graph). The same graph shows that fluorescent intensity forms a peak in the same wavelength band at high concentrations of analyte (labeled as “+ anal” in the graph), but the graph shows the peak intensity value is greater for the high concentration than for the low concentration. If this relationship were to persist over the analyte concentration range of interest, the bottom graph in the column, with calibration curve 171, would result. Here the intensity of the peak increases with analyte concentration over a concentration range of interest. In this example, the intensity in the wavelength band is the property of the fluorescent light used to determine the concentration of the analyte.

Similarly, and still referring to the graphs of FIG. 9 the top graph on the second column from the left shows that fluorescent intensity forms peaks in two separate wavelength bands (called band A and band B in the graph) at low concentrations of analyte (labeled as “− anal” in the graph). The intensity of the first peak (band A) is less than the intensity of the second peak (band B). The same graph shows that fluorescent intensity peaks in the same two separate wavelength bands at high concentrations of analyte (labeled as “+ anal” in the graph), but the intensity of the first peak (band A) is greater than the intensity of the second peak (band B). A ratio defined by dividing the intensity of the first peak (band A) by the intensity of the second peak (band B) is lower for low concentration of analyte and higher for the high concentration of analyte. If this relationship were to persist over the analyte concentration range of interest, the bottom graph in the column, with calibration curve 172, would result. Here, the ratio of the intensities of the two peaks increases with analyte concentration over a concentration range of interest. In other embodiments, the ratio of the intensities of the two peaks decreases with analyte concentration over a concentration range of interest. In these examples, the ratio of the intensities in the two bands is the property of the fluorescent light used to determine the concentration of the analyte.

As another example from FIG. 9, the top graph on the third column from the left shows that fluorescent intensity in a particular wavelength band decreases with time. The rate of decay is different for different concentrations of the analyte. The lifetime of the fluorescent response (τ) is given by a reciprocal of a slope of a line in the graph of the log of the intensity in the wavelength band against time. The lifetime τ is lower for low concentration of analyte and higher for the high concentration of analyte 192. If this relationship were to persist over the analyte concentration range of interest, the bottom graph in the column, with calibration curve 173, would result. Here, the lifetime (τ) increases with analyte concentration over a concentration range of interest. In this example, the lifetime τ is the property of the fluorescent light used to determine the concentration of the analyte.

In another example from FIG. 9, the top graph on the fourth column from the left shows electric field changes in time associated with a modulation frequency f. The method uses intensity-modulated light at some modulation frequency f that is much less than the optical frequency. Light modulation frequencies f typically range from 10 megaHertz (MHz, 1 MHz=10⁶ Hertz) to 300 MHz, but can be from 1 MHz to 10 gigaHertz (GHz, 1 GHz=10⁹ Hz), while optical frequencies are in the range of terahertz (THz, 1 THz=10¹² Hertz). The solid curve shows the timing (phase, ϕ) of the measured light modulations relative to that reference beam of light, with successive modulation peaks separated by 2π in phase for a wave period given by 1/f. The graph also shows an amplitude called a modulation for the wave which is related to the intensity. A measured field from emitted fluorescent light is given by the dashed curve and has a slightly different phase Δϕ and modulation Δm from the reference field. If the phase difference Δϕ or the modulation difference Δm from the reference changes with different concentrations of analyte 192, and if either or both were to persist over the analyte concentration range of interest, the bottom graph in the column would result. Here the phase difference Δϕ given by the dotted line increases with analyte concentration over a concentration range of interest, providing calibration curve 174a and the modulation difference Δm given by the solid line decreases with analyte concentration over a concentration range of interest, providing calibration curve 174b. In this example, either phase difference Δϕ or modulation difference Δm is the property of the emitted fluorescent light 163 used to determine the concentration of the analyte 192.

### Fluorescence Measurement System

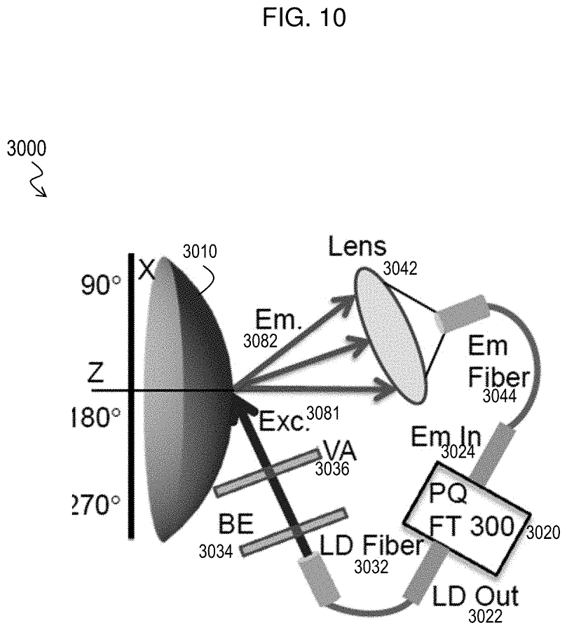

FIG. 10 illustrates an example measurement system 3000, according to an experimental embodiment. The system 3000 includes the probe material contact lens 3010 disposed on the cornea of a subject, with an axis of rotational symmetry defining the z direction, and the positive x direction at 90 degrees counterclockwise from the z axis. Tear fluid from the subject penetrates the contact lens 3010 and any analyte binds to the probe composition at the interface between the hydrogel channels and the silicone interstices.

An optical analyzer 3020, such as PicoQuant™ instrument (PQ FT 300 from PicoQuant™ Photonics International of West Springfield, Mass.) includes a pulsed laser diode (LD) that is energized to produce an laser diode (LD) source light that passes through LD output port 322 through optical fiber 3032 and optical couplers, such as beam expander (BE) and variable aperture (VA) 3036 to produce directional excitation light 3081 that impinges on the contact lens 3010. For example, the incident angle is selected to illuminate the contact lens 3010 at an angle to avoid direct incidence into the eye of the subject. The VA 3036 is used to illuminate part of the iris (1-2 mm diameter), the center outer surface of the pupil (1-2 mm), or the entire iris (about 10 mm) to obtain a maximal signal with minimal background.

The fluorescent emitted light, if any, emerges from the contact lens 3010 and is focused by an optical coupler, such as lens 3040, into the emission optical fiber 3044 where it is fed into the optical analyzer 3020 at optical input port 3024. The analyzer 3020 includes an LED source and control circuits, as well as an optical detector, analog to digital converter and Fourier analyzer. For example, the emission is collected with a 1-inch lens positioned about 1 inch from the eye, and focused into an optical fiber. The observation angle is close to perpendicular from the eye or slightly off the z axis. The emission is directed to the PicoQuant™ instrument for measurements of intensities, emission spectra or lifetimes. One can use light reflected or scattered from the eye as the timing reference for the lifetime analysis.

A digital input port (not shown) provides a digital signal from a separate processing system 380, such as a processor in a computer system 3100, chip set 3200 or mobile terminal 3300, or some combination, to control those components. A digital output port (not shown) outputs a digital signal carrying data that indicates the power fed into the LED and the spectrum of the received light. That output signal is received at the separate processing system 380 such as a processor in a computer system 3100, chip set 3200 or mobile terminal 3300, or some combination. At the processing system, the digital signal is used to determine the concentration of analyte in the tear fluid of the subject using a calibration curve appropriate for the probe material and detection method, as described above.

Computational Hardware Overview

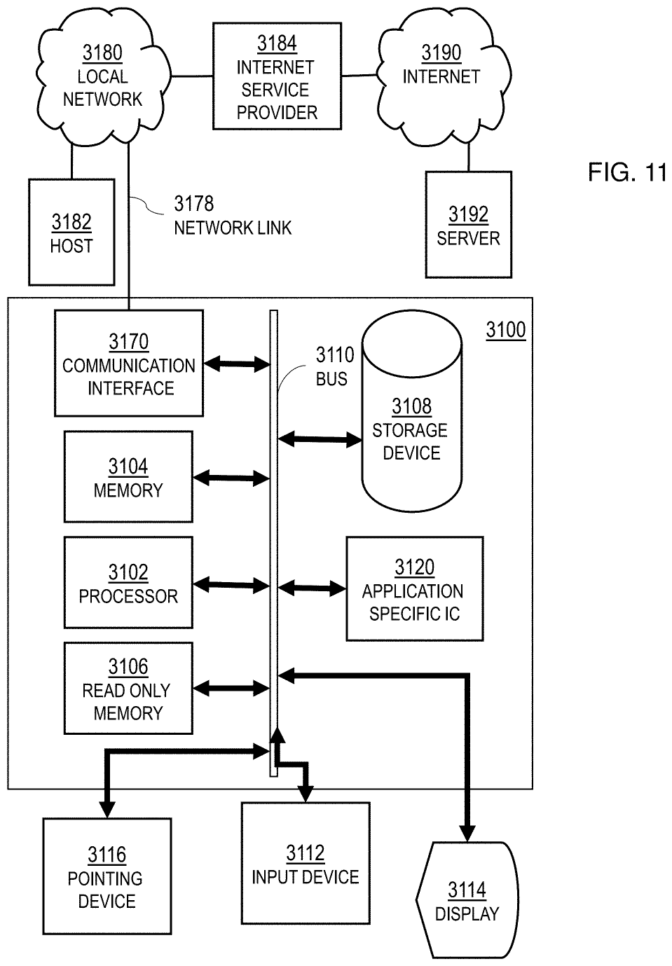

FIG. 11 illustrates a computer system 3100 upon which an embodiment of the invention may be implemented. Computer system 3100 includes a communication mechanism such as a bus 3110 for passing information between other internal and external components of the computer system 3100. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 3100, or a portion thereof, constitutes a means for performing one or more steps of one or more methods described herein.

A sequence of binary digits constitutes digital data that is used to represent a number or code for a character. A bus 3110 includes many parallel conductors of information so that information is transferred quickly among devices coupled to the bus 3110. One or more processors 3102 for processing information are coupled with the bus 3110. A processor 3102 performs a set of operations on information. The set of operations include bringing information in from the bus 3110 and placing information on the bus 3110. The set of operations also typically include comparing two or more units of information, shifting positions of units of information, and combining two or more units of information, such as by addition or multiplication. A sequence of operations to be executed by the processor 3102 constitutes computer instructions.

Computer system 3100 also includes a memory 3104 coupled to bus 3110. The memory 3104, such as a random access memory (RAM) or other dynamic storage device, stores information including computer instructions. Dynamic memory allows information stored therein to be changed by the computer system 3100. RAM allows a unit of information stored at a location called a memory address to be stored and retrieved independently of information at neighboring addresses. The memory 3104 is also used by the processor 3102 to store temporary values during execution of computer instructions. The computer system 3100 also includes a read only memory (ROM) 3106 or other static storage device coupled to the bus 3110 for storing static information, including instructions, that is not changed by the computer system 3100. Also coupled to bus 3110 is a non-volatile (persistent) storage device 3108, such as a magnetic disk or optical disk, for storing information, including instructions, that persists even when the computer system 3100 is turned off or otherwise loses power.

Information, including instructions, is provided to the bus 3110 for use by the processor from an external input device 3112, such as a keyboard containing alphanumeric keys operated by a human user, or a sensor. A sensor detects conditions in its vicinity and transforms those detections into signals compatible with the signals used to represent information in computer system 3100. Other external devices coupled to bus 3110, used primarily for interacting with humans, include a display device 3114, such as a cathode ray tube (CRT) or a liquid crystal display (LCD), for presenting images, and a pointing device 3116, such as a mouse or a trackball or cursor direction keys, for controlling a position of a small cursor image presented on the display 3114 and issuing commands associated with graphical elements presented on the display 3114.

In the illustrated embodiment, special purpose hardware, such as an application specific integrated circuit (IC) 3120, is coupled to bus 3110. The special purpose hardware is configured to perform operations not performed by processor 3102 quickly enough for special purposes. Examples of application specific ICs include graphics accelerator cards for generating images for display 3114, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 3100 also includes one or more instances of a communications interface 3170 coupled to bus 3110. Communication interface 3170 provides a two-way communication coupling to a variety of external devices that operate with their own processors, such as printers, scanners and external disks. In general, the coupling is with a network link 3178 that is connected to a local network 3180 to which a variety of external devices with their own processors are connected. For example, communication interface 3170 may be a parallel port or a serial port or a universal serial bus (USB) port on a personal computer. In some embodiments, communications interface 3170 is an integrated services digital network (ISDN) card or a digital subscriber line (DSL) card or a telephone modem that provides an information communication connection to a corresponding type of telephone line. In some embodiments, a communication interface 3170 is a cable modem that converts signals on bus 3110 into signals for a communication connection over a coaxial cable or into optical signals for a communication connection over a fiber optic cable. As another example, communications interface 3170 may be a local area network (LAN) card to provide a data communication connection to a compatible LAN, such as Ethernet. Wireless links also may be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 3170 sends and receives electrical, acoustic or electromagnetic signals, including infrared and optical signals that carry information streams, such as digital data.

The term computer-readable medium is used herein to refer to any medium that participates in providing information to processor 3102, including instructions for execution. Such a medium may take many forms, including, but not limited to, non-volatile media, volatile media and transmission media. Non-volatile media include, for example, optical or magnetic disks, such as storage device 3108. Volatile media include, for example, dynamic memory 3104. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 3102, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 3102, except for carrier waves and other signals.

Logic encoded in one or more tangible media includes one or both of processor instructions on a computer-readable storage media and special purpose hardware, such as ASIC 313120.

Network link 3178 typically provides information communication through one or more networks to other devices that use or process the information. For example, network link 3178 may provide a connection through local network 3180 to a host computer 3182 or to equipment 3184 operated by an Internet Service Provider (ISP). ISP equipment 3184 in turn provides data communication services through the public, world-wide packet-switching communication network of networks now commonly referred to as the Internet 3190. A computer called a server 3192 connected to the Internet provides a service in response to information received over the Internet. For example, server 3192 provides information representing video data for presentation at display 3114.

The invention is related to the use of computer system 3100 for implementing the techniques described herein. According to one embodiment of the invention, those techniques are performed by computer system 3100 in response to processor 3102 executing one or more sequences of one or more instructions contained in memory 3104. Such instructions, also called software and program code, may be read into memory 3104 from another computer-readable medium such as storage device 3108. Execution of the sequences of instructions contained in memory 3104 causes processor 3102 to perform the method steps described herein. In alternative embodiments, hardware, such as application specific integrated circuit 3120, may be used in place of or in combination with software to implement the invention. Thus, embodiments of the invention are not limited to any specific combination of hardware and software.

The signals transmitted over network link 3178 and other networks through communications interface 3170, carry information to and from computer system 3100. Computer system 3100 can send and receive information, including program code, through the networks 3180, 3190 among others, through network link 3178 and communications interface 3170. In an example using the Internet 3190, a server 3192 transmits program code for a particular application, requested by a message sent from computer 3100, through Internet 3190, ISP equipment 3184, local network 3180 and communications interface 3170. The received code may be executed by processor 3102 as it is received or may be stored in storage device 3108 or other non-volatile storage for later execution, or both. In this manner, computer system 3100 may obtain application program code in the form of a signal on a carrier wave.

Various forms of computer readable media may be involved in carrying one or more sequence of instructions or data or both to processor 3102 for execution. For example, instructions and data may initially be carried on a magnetic disk of a remote computer such as host 3182. The remote computer loads the instructions and data into its dynamic memory and sends the instructions and data over a telephone line using a modem. A modem local to the computer system 3100 receives the instructions and data on a telephone line and uses an infra-red transmitter to convert the instructions and data to a signal on an infra-red a carrier wave serving as the network link 3178. An infrared detector serving as communications interface 3170 receives the instructions and data carried in the infrared signal and places information representing the instructions and data onto bus 3110. Bus 3110 carries the information to memory 3104 from which processor 3102 retrieves and executes the instructions using some of the data sent with the instructions. The instructions and data received in memory 3104 may optionally be stored on storage device 3108, either before or after execution by the processor 3102.

### 5. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are described. All publications mentioned herein, are incorporated by reference in their entirety; nothing herein is to be construed as an admission that the invention is not entitled to antedate such disclosure by virtue of prior invention.

### Example 1. Methods and Technical Discussion

Fluorescence Spectroscopic Measurements. Fluorescence intensities and intensity decays were measured using a Varian™ Eclipse 4 spectrofluorometer and a FluoTime300 instrument from PicoQuant™ (Berlin, Germany) and analyzed with the EasyTau™ software. The excitation source was a 473 nm laser diode with a repetition rate of 40 MHZ and a pulse width less than 100 ps. In other embodiments, the excitation source for SG-PL was at 495 nm from a Solea™ supercontinuum laser (PicoQuant™, Germany) and a 375 nm laser diode (PDL 375, PicoQuant™), a repetition rate of 40 MHz, and a pulse width less than 100 ps. Some intensity and lifetime measurements for SG-PL (as indicated) were performed using Alba V confocal fluorescence lifetime microscopy, FLIM (ISS, Urbana, Ill.), with excitation source of a 473 nm laser diode with a repetition rate of 40 MHz and a pulse width near 250 ps.

Intensity decays were measured at the emission maximum with the band of 10 to 20 nm, analyzed in terms of the multi-exponential model:

I(t)=Σαᵢ exp(−t/tᵢ) (equation 1)

where tᵢ are the individual decay times and αᵢ the initial time-zero amplitudes. The mean decay time can be represented in two ways. The average lifetime is given by:

$${τ}_{f} = \frac{Σ {α}_{i} {τ}_{1}^{2}}{Σ {α}_{i} {τ}_{i}^{2}} = Σ {f}_{i} {τ}_{f} \qquad ( equation 2 )$$

where Σαᵢ=1.0 and Σfᵢ=1.0. The term fᵢ=αᵢ τᵢ is proportional to the fractional contribution of each component to the total intensity decay. The term τᵢ/Σαᵢ τᵢ is the fractional contribution to the steady state emission. Here, we use the amplitude-weighted lifetime given by

$${τ}_{α} = \frac{Σ {α}_{i} {t}_{i}}{Σ {α}_{i}} , \qquad ( equation 3 )$$

which is appropriate when the emission is from a single fluorophore with a changing quantum yield. For a single exponential decay, the lifetime is given by τ=1.0/(kₙᵣ+Γ) and the quantum yield is given by Q=Γ/(kₙᵣ+Γ) where kₙᵣ is the sum of all non-radiative decay rates and Γ is the radiative decay rate. The value of Γ is determined by the molecular extinction coefficient that remains constant under most conditions. For a single type of fluorophore with a constant value of Γ the normalized αᵢ values represent the molecular fraction of each decay time component. The value of αᵢ and τᵢ were determined by non-linear least squares fitting using PicoQuant™ software.

Three decay time components were required for the intensity decays. Fluorescence anisotropy decays were determined by individual measurements of the polarized intensity decays with corrections for the G-factor. Resolution of multi-exponential decays is not necessary. The average lifetime can be used as a single measurement.

Fluorescence Microscopy Measurements. Fluorescence microscopy and fluorescence lifetime imaging microscopy (FLIM) were performed using a laser scanning confocal microscope from ISS (Champaign, Ill.) with a large area (1 cm×lcm) stage scanner. The excitation source was a 473 nm pulsed laser diode, with observation at 560 nm (50 nm bandwidth) and a 20× objective.

Sodium Binding Affinity. The sodium affinity of the labeled lenses can be described in terms of the dissociation constant. The dissociation reaction for the probe complexed with Na+ can be written as

P—Na⁺↔P+Na⁺ (equation 4)

where P is the free probe with no bound sodium in the dissociation constant K_{D} is given. The ratio of sodium free and sodium bound probe is given by

$${K}_{D} = \frac{[ P ] [ {Na}^{+} ]}{[ P - {Na}^{+} ]} \qquad ( equation 5 )$$

The binding affinity is given as the mid-point (MP) of the ion-dependent response where the spectral change is 50% complete. In isotropic media, the value of MD is a measure of KD. A lager value of KD corresponds to a weaker binding affinity and a lower value of K_{D} indicates stronger affinity for Na+ ions. The definition of K demonstrates the value of the amplitude weighted lifetimes, which represents the fraction of SG-PL in the free or Na+-bound forms.

In some embodiments, the binding affinities are described as the sodium concentration at the mid-point of the spectral responses, which is comparable to the dissociation constant of the probe-sodium complex. The ratio of sodium free and sodium bound probe is given by

$$\frac{[ P ]}{[ P - {Na}^{+} ]} = \frac{{K}_{D}}{[ {Na}^{+} ]} \qquad ( equation 6 )$$

Quenching by Chloride. Collisional quenching occurs, for example, when SPQ-C18 (also referred to as OD-MQB (N-Octadecyl-6-methoxyquinolinium bromide)) has a diffusion collision with a chloride ion. The intensities (I) and lifetimes (6) are analyzed using the Stern-Volmer equation,

$$\begin{matrix}\frac{{F}_{0}}{F} = 1 + K [ {CL}^{-} ] & ( equation 7 ) \\ \frac{{τ}_{0}}{τ} = 1 + K [ {Cl}^{-} ] & ( equation 8 )\end{matrix}$$

where F₀ and τ₀ are the intensities and lifetimes in the absence of chloride, and K is the quenching constant and the inverse of the [Cl⁻] needed for 50% quenching. For collisional quenching, the value of F₀/F and τ₀/τ are typically the same. In solution, in the absence of lenses, the value of K=k_{q} τ₀ can be used to calculate the biomolecular quenching constant k_{q} and the quenching efficiency. In the lenses, the [Cl−] may not be the same as in the bulk solution, but k_{q} in the lenses is estimated by assuming the same concentration of chloride around the SPQ moiety in the lenses and in bulk solutions.

Light sources and detectors for fluorescence. Many solid-state LEDs and laser diodes (LO) are available for most wavelengths above 260 nm and extending to over 900 nm in the NIR. Pulsed laser diodes (without frequency doubling) are available down to 375 nm. Wavelengths below 400 nm are absorbed by the cornea and do not reach the retina. Shorter wavelengths are blocked by many contact lenses. Therefore, the structures of most known ion-sensitive fluorophores can be used as starting points for making probes that are functional in SiHG lenses.

Fluorescence and FLIM Images of the EL-CL. The rapidly evolving technology for CMOS detector arrays enables hand-held battery powered EL-CL reading devices that can measure the intensities and lifetimes in different locations on a contact lens (see FIG. 1B). Charge-coupled devices (CCDs) are rapidly being replaced by CMOS detector arrays (CDAs) that require 100-fold less power than CCDs. CDAs have high sensitivity and frame rates, have been used for live cell imaging and single molecule detection. CDAs are capable of measuring nanosecond decay times and therefore useful for fluorescence lifetime imaging microscopy (FLIM). A new CDA is now available that obtains 30 images using the time-of-flight from camera-to-surface and back. Since the distance resolution appears to be below 1 inch the time resolution must be below 1 ns.

Iris Tracking Technology. In embodiments configured for iris imaging (e.g., for iris tracking), the EL-CL can contain multiple locations that provide known emission intensities and/or lifetimes for calibration, and thus be self-calibrating. Thus, the contact lens can be configured to measure multiple ions or multiple spots on a lens even when the exact iris location is not known and the iris may be moving. For example, typical imaging devices can be used for identification of individuals by imaging the iris. The availability of point-of-care measurements of tear electrolytes can provide an immediate health benefit for individuals with DED.

Absence of Electronics in Lenses. Typical lenses contain electronic circuits and antennas to capture energy to power the device. As a result they are likely to be expensive, not suitable for one day use and available in only a single type of contact lens polymer. The lenses disclosed here does not require any electronics in the lenses. Addition of probes is likely to be inexpensive compared to typical devices and the labeled lenses will be generally compatible with one-day use of contact lenses.

Selection of Probe Structure.

The structures of the sodium sensitive fluorophores were selected in consideration of the composition of the lens's SiHG polymers. The Comfilcon A lenses were designed for high permeability to both oxygen and the ions present in tears. The diffusion of polar ions is facilitated by a semi-interpenetrating polymers network (IPN) that contains continuous water channels from the front to back surfaces of the lenses (see FIG. 2). Oxygen transport occurs through the silicone-rich regions. Oxygen is almost 10-fold more soluble in non-polar solvents than in water, and the permeability silicone to oxygen is 100-fold higher than for other organic polymers. In previous publications, the presence of non-polar regions in SiHG lenses was demonstrated using polarity-sensitive fluorophores. The structure of SG-C16 contains two non-polar alkyl chains that bind at the water-silicone interface. Lysophosphatidyl ethanolamine was used in SG-LPE because it is a natural biomolecule and amphipathic lipids are used to increase the wettability of the hydrophobic silicone regions. Lysolipids differ from typical phospholipids by the loss of one alkyl chain. As a result, lyso lipids form micelles that are 2-4 nm in diameter instead of the larger 200-400 nm diameters of liposomes. The pore size of a SiHG lens is thought to be around 50 nm, so that smaller micelles would diffuse into the water channels and facilitate labeling lenses with the hydrophobic sodium probes. SG-PL contains poly-L-lysine (see Chemical Scheme 1, FIG. 7), which is widely used to decrease the hydrophobicity of glass, PDMS and silicone, and can be expected to bind at the interface region of the lenses. Polylysine also could bind the non-polar regions of SiHG. The monomers used in making SiHG polymers include regions with partially oxidized carbon atoms, carboxyl groups and cross-liners. These groups can contribute a negative charge to this region of the lens and possible electrostatic binding to polylysine. In addition, electrostatic binding could become important for probe binding to newer and technically emerging low-silicone contact lenses.

### Example 2. Synthesis of a Sodium-Sensitive Fluorophore Probe Compound

Three sodium-sensitive fluorophores were produced. Each is a derivative of Sodium Green (SG). See Chemical Scheme 1, in FIG. 7.

For compound SG-C16, R=1-hexadecyl amine. For compound SG-LPE, R=lyso phosphatidyl-ethanolamine. For compound SG-PL, R=poly-L-lysine. SG-C16 was prepared using NHS+EOG-activated amide bond formation with two 1-hexadecylamines. A second hydrophobic sodium probe was prepared using the same method to link two molecules of 1-oleoyl-2-hydroxy-sn-glycero-3-phosphoethanolamine (18:1 Lyso-PE, Avanti Polar Lipids Inc.), forming SG-LPE. The third sodium probe was SG linked to poly-l-lysine (Sigma-Aldrich™, MW=70-150 kDa). A typical reaction was performed as follows. A solution of SG (tetramethylammonium) salt (cell impermeant, Thermo Fisher Scientific™ 1 mg, 6.0×10⁻⁴ mmol), in 2 ml dimethylformamide (DMF) was prepared and mixed with N-(3-dimethylaminopropyl)-N′-ethylcarbodiimide hydrochloride (EDC, 0.34 mg, 2.0×10·³ mmol) and N-hydroxysuccinimide (NHS, 0.2 mg, 2.0×10.3 mmol). The solution was stirred overnight at room temperature under an inert atmosphere. To the reaction mixture either 1-hexadecylamine (0.17 mg, 7.2×10⁴ mmol) or lyso PE (0.38 mg, 7.2×10⁴ mmol) was added and the reaction continued for an additional 6 hours. For SG-PL, we used 0.15 ml of 0.01% poly-l-lysine in water and continued reaction for additional 6 hours. 0.015 ml aliquots of as prepared reaction mixtures were diluted in 2 ml of water to bind the probes into the contact lens. The probe concentrations in the solution used for labeling were about 1 μM. The solution contains the probe of choice, for example SG-C16, in water at about 1 mM. Typically, about 2 to 3 mL of the solution was used for a single lens, enough solution to fully cover the lens. The labeled lenses were extensively washed with deionized water to eliminate any loosely bound probe from the lenses before being used for ion responsive studies. Unless stated otherwise, all work was performed in 20 mM MOPS buffer, 8 mM KCl, pH 7.2, at room temperature, with variable concentrations of NaCl. The emission spectra and lifetimes data for each sodium concentrations were result of five separate experiments.

In one embodiment, the sodium-sensitive fluorophores is a conjugate of Sodium Green (SG) with poly-L-lysine (PL). SG-PL was prepared using activated amide bond formation (to 1 mg of SG (6.6×10⁻² mmol) (tetramethylammonium salt, cell impermeant) in 2 mL of dimethylformamide (DMF) and mixed with N-(3-dimethylaminopropyl)-N-ethylcarbodiimide hydrochloride (EDC, 0.34 mg, 2.0×10-3 mmol) and N-hydroxysuccinimide (NHS, 0.2 mg, 2.0×10-3 mmol). The solution was stirred overnight at room temperature under an inert atmosphere. 0.015 mL aliquots of prepared reaction mixture were diluted in 2 mL of water to bind the probes to the contact lens.

### Example 3. Synthesis of a Chloride-Sensitive Fluorophore Probe

SPQ-C18 was synthesized using 6-methoxy quinolinium (SPQ) and 1-bromooctadecane (C18) as described in the art. Lenses were labeled by incubation with 3 ml of a 1 μM solution of SPQ-C18 in methanol-water. SPQ-C18 bound rapidly to the Biofinity™ lenses. SPQ-C18 binding to the MyDay™ lenses was slower and required several days of incubation in the labeling solution. Unless stated otherwise, all spectral measurements conducted for the present study were performed in 20 mM MOPS buffer, 8 mM KCl, pH 7.2, at room temperature. See Table 11 for structure.

### Example 4. Labeling of Biofinity™ Lenses with Sodium Probes

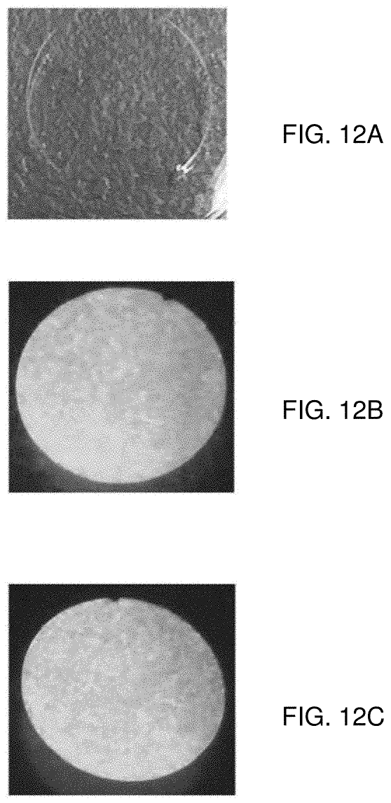

The lenses each were labeled by incubation in 2 ml of a 1 μm solution of probes. The rates of probe uptake into the lenses were not studied in detail but SG-PL appeared to bind the most rapidly. After an hour, the lenses were roughly equally intense with the three probes. We did not determine the amounts of probe bound to the lenses or the amount of probe remaining in the labeling solution. After labeling, the lenses were washed several times in 3 ml of probe-free buffer, which is a 200-fold larger volume than the volume of a contact lens (approximately 15 μl). This washing resulted in about half of the intensity being removed from the lenses. This result is consistent with unbound probe in the aqueous IPN channels being removed by sensing. After this initial decrease in intensity, the lenses retained the same brightness for a period of weeks when stored in 3 ml of probe-free buffer. Each of the three sodium probes (SG-C16, SG-LPE, and SG-PL) were found to bind strongly to the Biofinity™ lenses, resulting in uniform emission across the lens that was visible in a darkened room (FIG. 12). This figure presents photographs of SG-PL in a Biofinity™ contact lens in room lights (FIG. 12A) and with diffuse 473 nm illumination for 0 (FIG. 12B) and 150 mM (FIG. 12C) NaCl.

### Example 5. Binding of the Sodium Probe to Contact Lenses

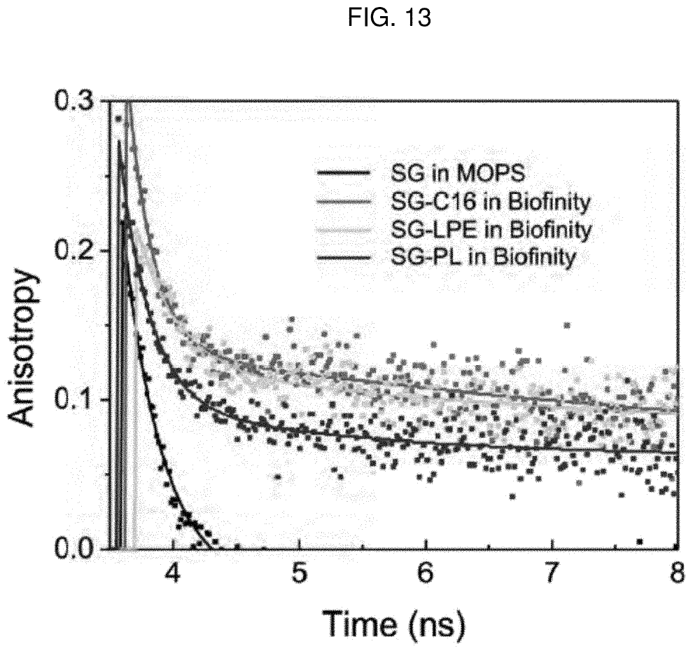

Sodium probe binding to the lenses was demonstrated by measurements of the anisotropy decays. For the free probe SG, the anisotropy decayed quickly to zero (FIG. 13). The three sodium probes all displayed in lenses a rapid initial anisotropy decay followed by a long correlation time that did not decay to zero during the 1-3 ns lifetime of the fluorophores. Similar anisotropy decays have been observed for numerous fluorophores bound to large proteins or membranes. These results (see FIG. 13) are consistent with the ion-binding region of the probes being free to rotate in the aqueous channels, but the overall global rotation of the probes limited by the interfacial regions of the lenses.

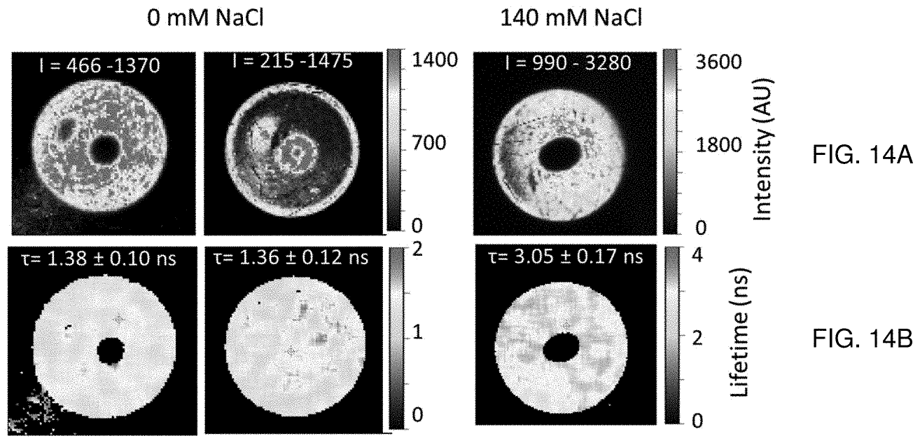

Strong and essentially complete binding of SG-C16 to the Comfilcon A lenses was confirmed by the confocal fluorescence intensity images shown in FIG. 14. The images show confocal intensity and lifetime images of SG-C 16 in Biofinity™ contact lenses for 0 and 140 mM NaCl. Images at 0 mM NaCl were acquired at two different focal planes.

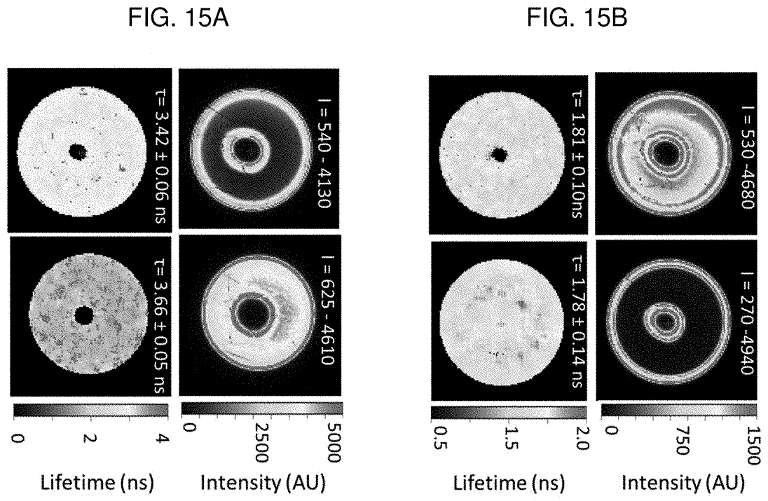

No significant intensity was observed outside the lens with 0 mM or 140 mM NaCl. The confocal images are different because they were measured at different focal planes. Importantly, the FLIM images show lifetimes that were essentially identical in all regions of the lens. The lifetime changed uniformly from 1.37 ns in the absence of sodium to 3.05 ns in the presence of sodium, which indicates that any region of the lens can be used for sodium sensing. Similar results were obtained for SG-LPE (FIG. 15) and SG-PL (FIG. 16), which also show complete binding of the probes to the lenses and an increase in lifetime in the presence of sodium. These results demonstrate that several different approaches can be used to bind ion-sensitive fluorophores to SiHG contact lenses. For FIG. 15, confocal intensity and lifetime images of SG-LPE are shown in Biofinity™ contact lenses. The images at 0 mM NaCl (top) were acquired at two different focal planes. Images at 100 and 240 mM NaCl were acquired at the same focal plane. For FIG. 16, confocal intensity and lifetime images of SG-PL are shown in Biofinity™ contact lenses with 0 and 100 mM NaCl. Images at 0 mM NaCl were acquired at two different focal planes.

### Example 6. Binding of the Chloride Probe to Contact Lenses

The probe concentrations in the doping solution were about 1 μM. SL-PL and SPQ-C18 were found to bind rapidly to the Biofinity™ lenses, within one hour. Probe uptake was noticeably slower with the MyDay™ lens, which were left in the labeling solution for several days. The labeled lenses were extensively washed with ionized water to eliminate any loosely bound probe from the lenses before use in the ion responsive studies.

### Example 7. Sodium Response of the Labeled Contact Lens

The SG probe is based on an aza crown ether with two nitrogen atoms. This structure was originally designed to obtain a suitable high affinity probe for detecting sodium with a binding constant (mid-point) near 6-10 mM (the physiological range for intracellular sodium). However, the sodium concentration in tears is near 120 mM and comparable to the concentration in blood. Therefore, this probe would not be expected to operate in a system to detect sodium in tears.

In embodiments of the invention, it was discovered that the sodium affinity of SG, when bound to contact lenses, actually decreased for two reasons. First. the aza crown ether and attached fluorophores could be partially buried in silicone regions of the lenses and less accessible to the aqueous phase. Second, the parent fluorophore SG contains two negatively charged carboxyl groups that may contribute to the sodium binding affinity.

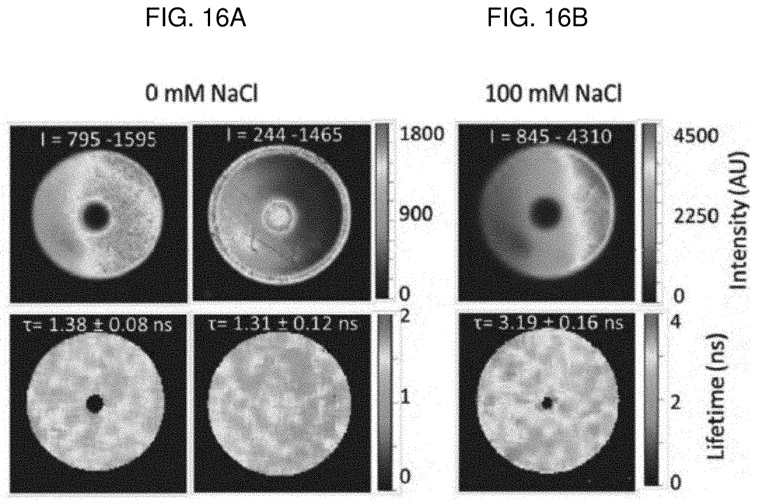

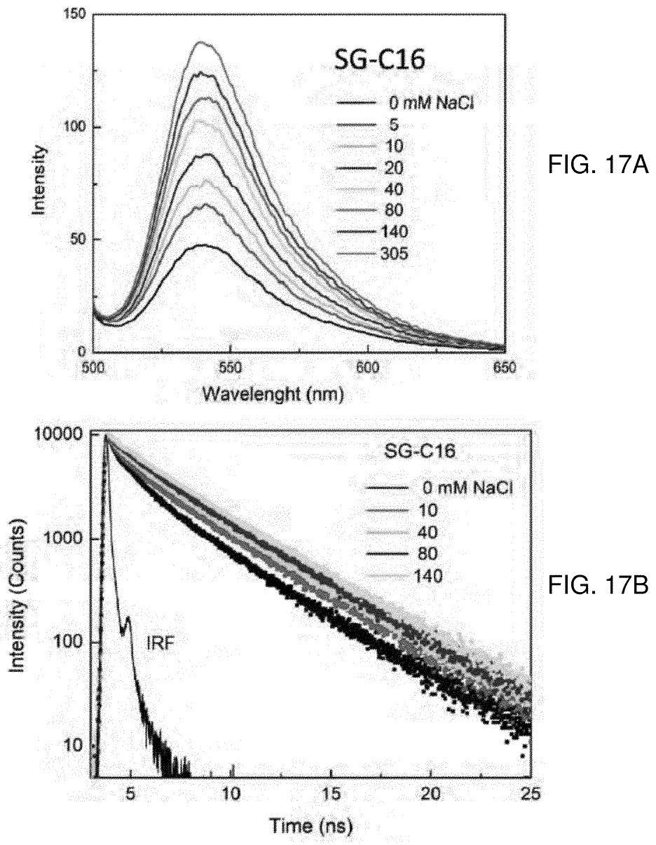

This role of carboxyl groups for increased sodium affinity of SG is supported by the weaker sodium binding of CoroNa™ Green, CoroNa™ Red and Asanti NaTrium™ Green that do not have negative carboxyl groups near the sodium binding sites. Negatively charged carboxyl groups are not present in the three preferred sodium probes. FIG. 17 shows the effects of sodium on the fluorescence intensities and lifetimes of SG-C16 in Biofinity™ lenses. This figure shows the sodium-dependent emission spectra (FIG. 17A) and intensity decays (FIG. 17B) of SG-C 16 in Biofinity™ contact lenses.

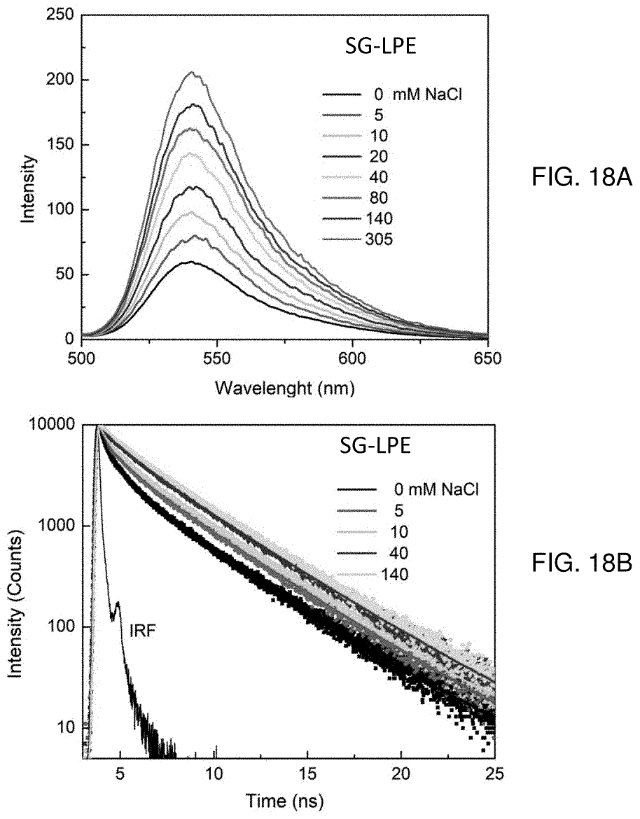

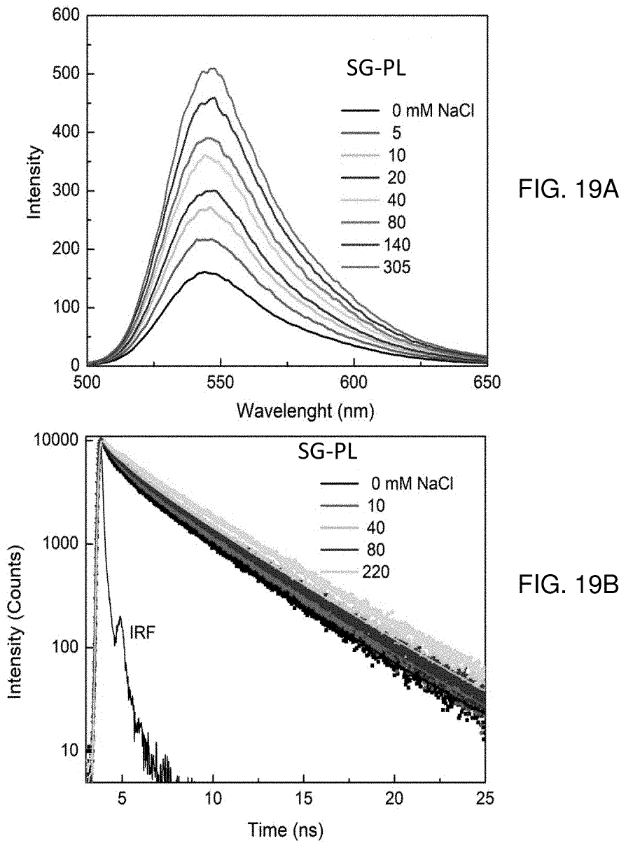

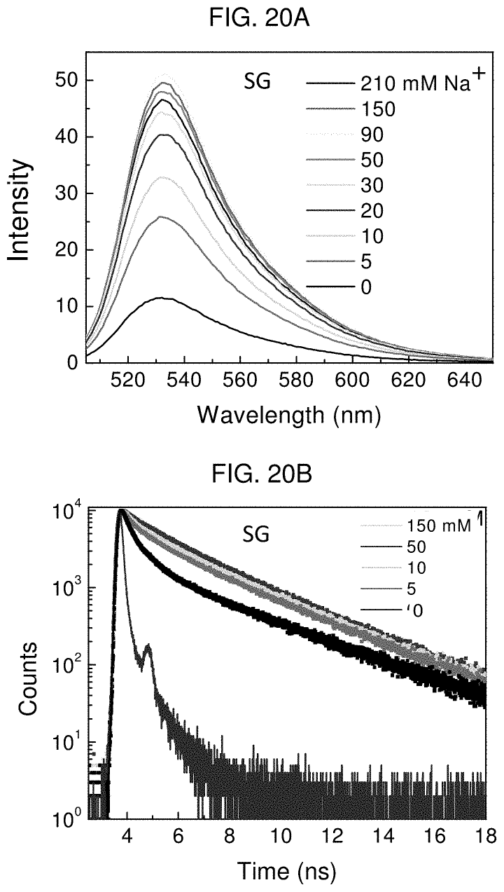

The fluorescence intensities increased about 3-fold as the sodium concentration is increased from O to 150 mM. The emission spectra do not change shape or wavelength in a way that would make it difficult to use the intensities alone to determine tear sodium concentrations. Addition of a fluorophore not sensitive to sodium would allow wavelength-ratiometric sensing using SG-C16. Importantly, the rate of the intensity decay decreases (FIG. 17) and the fluorescence lifetime increases, which allows lifetime-based sensing of sodium. Similar changes in intensity and lifetime were observed for SG-LPE and SG-PL. See FIG. 18 and FIG. 19, which show sodium-dependent emission spectra (FIG. 18A and FIG. 19A) and intensity decays (FIG. 18B and FIG. 19B) of SG-LPE and SG-PL, respectively, in Biofinity™ contact lenses. See also, for comparison, FIG. 20A and FIG. 20B for sodium-dependent emission spectra and intensity decays respectively, Sodium Green (SG) in 20 mM MOPS buffer with 8 mM KCl.

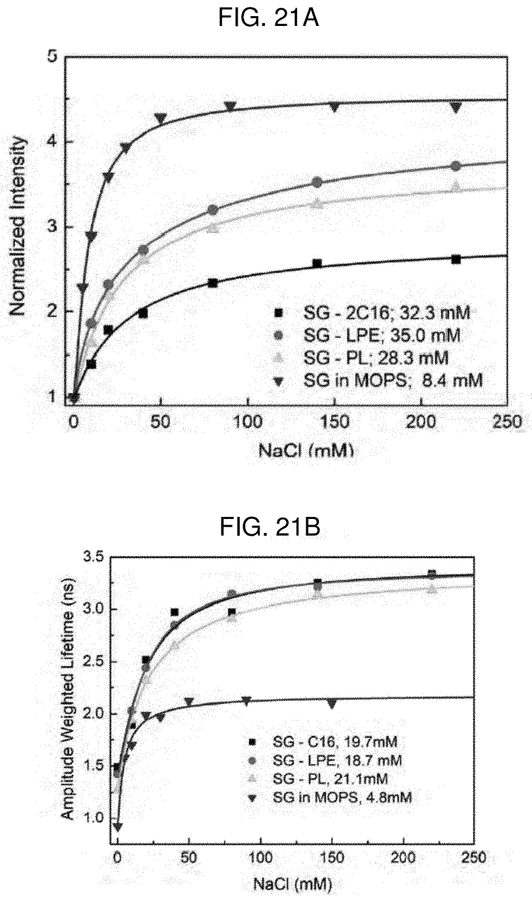

Intensity and lifetime sodium titration curves for SG in buffer and the three probes (in lens) are shown in FIG. 21. The figure shows sodium-dependent intensities and lifetimes for SG in MOPS buffer and the three sodium probes in Biofinity™ lenses. Lifetime measurements were performed on Fluotime 300 instrument. Numerical values correspond to the mid points of sodium-dependent responses.

The multi-exponential analyses are summarized in Tables 7-10. below. SG in buffer displayed a binding affinity near 6.5 mM, which is too high for measurements of sodium in tears.

TABLE 7
SG-C16 in Biofinity ™ Lens (20 mM MOPS, pH 7.3).

| NaCl | τ₁ | τ₂ | τ₃ | | | | | | | τ_{α} | τ_{f} |
|---|---|---|---|---|---|---|---|---|---|---|---|
| (mM) | (ns) | (ns) | (ns) | α₁ | α₂ | α₃ | f₁ | f₂ | f₃ | (ns) | (ns) |
| 0 | 0.26 | 1.28 | 3.54 | 0.376 | 0.317 | 0.306 | 0.062 | 0.257 | 0.681 | 1.59 | 2.76 |
| 5 | 0.29 | 1.33 | 3.73 | 0.378 | 0.198 | 0.424 | 0.056 | 0.135 | 0.810 | 1.95 | 3.21 |
| 10 | 0.38 | 1.66 | 3.79 | 0.351 | 0.229 | 0.420 | 0.063 | 0.181 | 0.757 | 2.10 | 3.19 |
| 20 | 0.36 | 1.76 | 3.86 | 0.284 | 0.218 | 0.499 | 0.042 | 0.160 | 0.798 | 2.41 | 3.37 |
| 40 | 0.51 | 2.02 | 3.83 | 0.260 | 0.244 | 0.495 | 0.053 | 0.196 | 0.751 | 2.52 | 3.30 |
| 80 | 0.42 | 1.75 | 3.81 | 0.161 | 0.359 | 0.580 | 0.025 | 0.166 | 0.809 | 2.73 | 3.38 |
| 140 | 0.39 | 1.63 | 3.82 | 0.183 | 0.168 | 0.649 | 0.025 | 0.097 | 0.878 | 2.82 | 3.52 |

TABLE 8
SG-LPE in Biofinity ™ Lens (20 mM MOPS, pH 7.3).

| NaCl | τ₁ | τ₂ | τ₃ | | | | | | | τ_{α} | τ_{f} |
|---|---|---|---|---|---|---|---|---|---|---|---|
| (mM) | (ns) | (ns) | (ns) | α₁ | α₂ | α₃ | f₁ | f₂ | f₃ | (ns) | (ns) |
| 0 | 0.25 | 1.10 | 3.58 | 0.524 | 0.256 | 0.220 | 0.110 | 0.235 | 0.655 | 1.20 | 2.63 |
| 5 | 0.25 | 1.18 | 3.64 | 0.426 | 0.249 | 0.325 | 0.067 | 0.186 | 0.748 | 1.58 | 2.96 |
| 10 | 0.30 | 1.35 | 3.70 | 0.378 | 0.240 | 0.382 | 0.061 | 0.175 | 0.764 | 1.85 | 3.08 |
| 20 | 0.37 | 1.57 | 3.74 | 0.328 | 0.231 | 0.441 | 0.057 | 0.169 | 0.773 | 2.13 | 3.18 |
| 40 | 0.39 | 1.67 | 3.78 | 0.262 | 0.244 | 0.494 | 0.043 | 0.171 | 0.786 | 2.38 | 3.27 |
| 80 | 0.41 | 1.94 | 3.97 | 0.210 | 0.252 | 0.538 | 0.032 | 0.181 | 0.788 | 2.71 | 3A9 |
| 140 | 0.43 | 1.63 | 3.75 | 0.189 | 0.221 | 0.590 | 0.031 | 0.136 | 0.833 | 2.65 | 3.36 |

TABLE 9
SG-PL in Biofinity ™ Lens (20 mM MOPS, pH 7.3).

| NaCl | τ₁ | τ₂ | τ₃ | | | | | | | τ_{α} | τ_{f} |
|---|---|---|---|---|---|---|---|---|---|---|---|
| (mM) | (ns) | (ns) | (ns) | α₁ | α₂ | α₃ | f₁ | f₂ | f₃ | (ns) | (ns) |
| 0 | 0.24 | 1.12 | 3.60 | 0.473 | 0.267 | 0.260 | 0.083 | 0.221 | 0.696 | 1.35 | 2.77 |
| 5 | .047 | 1.86 | 3.85 | 0.417 | 0.221 | 0.363 | 0.097 | 0.205 | 0.697 | 2.00 | 3.11 |
| 10 | .044 | 1.62 | 3.86 | 0.381 | 0.209 | 0.410 | 0.080 | 0.162 | 0.758 | 2.09 | 3.22 |
| 20 | .069 | 2.74 | 4.49 | 0.387 | 0.414 | 0.199 | 0.116 | 0.495 | 0.390 | 2.30 | 3.19 |
| 40 | 0.38 | 1.54 | 4.02 | .0239 | 0.248 | 0.514 | 0.036 | 0.151 | 0.814 | 2.54 | 3.51 |
| 80 | 0.69 | 2.17 | 3.99 | 0.262 | 0.232 | 0.506 | 0.067 | 0.186 | 0.747 | 2.70 | 3.43 |
| 140 | .047 | 1.75 | 4.21 | 0.200 | 0.198 | 0.602 | 0.032 | 0.117 | 0.852 | 2.98 | 3.81 |
| 220 | .040 | 1.86 | 4.23 | 0.149 | 0.241 | 0.610 | 0.019 | 0.145 | 0.836 | 3.09 | 3.81 |

TABLE 10
SG in 20 mM MOPS (8 mM KCl).

| NaCl | τ₁ | τ₂ | τ₃ | | | | | | | τ_{α} | τ_{f} |
|---|---|---|---|---|---|---|---|---|---|---|---|
| (mM) | (ns) | (ns) | (ns) | α₁ | α₂ | α₃ | f₁ | f₂ | f₃ | (ns) | (ns) |
| 0 | 0.36 | 1.03 | 3.59 | 0.703 | 0.155 | 0.142 | 0.272 | 0.174 | 0.554 | 0.92 | 2.27 |
| 5 | 0.39 | 1.44 | 3.28 | 0.439 | 0.244 | 0.317 | 0.111 | 0.223 | 0.666 | 1.57 | 2.55 |
| 10 | 0.40 | 1.43 | 3.22 | 0.341 | 0.276 | 0.383 | 0.078 | 0.224 | 0.699 | 1.77 | 2.60 |
| 20 | 0.57 | 2.00 | 3.39 | 0.324 | 0.353 | 0.323 | 0.094 | 0.355 | 0.551 | 1.99 | 2.63 |
| 30 | 0.54 | 1.72 | 3.27 | 0.255 | 0.349 | 0.396 | 0.068 | 0.295 | 0.638 | 2.03 | 2.63 |
| 50 | 0.66 | 1.95 | 3.35 | 0.259 | 0.380 | 0.361 | 0.081 | 0.349 | 0.570 | 2.12 | 2.64 |
| 90 | 0.64 | 1.93 | 3.36 | 0.242 | 0.416 | 0.342 | 0.074 | 0.380 | 0.545 | 2.11 | 2.61 |
| 150 | 0.81 | 2.48 | 4.38 | 0.305 | 0.605 | 0.091 | 0.116 | 0.699 | 0.185 | 2.15 | 2.64 |
| 210 | 0.70 | 1.88 | 3.29 | 0.224 | 0.424 | 0.352 | 0.074 | 0.377 | 0.549 | 2.12 | 2.57 |

The sodium affinity was decreased for the three probes bound to Biofinity™ lenses to values near 30 mM. Changes in intensity and lifetime continue to the physiological sodium range in tears of 120 mM, but the probes are mostly saturated and the changes smaller above 100 mM sodium. We expect the sodium probes for the final EL-CL can display slightly weaker sodium binding. This can be accomplished by modification of the sodium binding structure by the use of non-azo crown ethers or mono-azo crown ethers. Additionally, the sodium affinity may be dependent on the type of contact lens polymer, which will be shown in a future report. The essential point is that sodium concentrations can be determined with labeled contact lenses and the sodium binding affinities can be adjusted to match the physiological sodium concentration in tears.

### Example 8. Reversibility and Effects of Potential Interfering Proteins

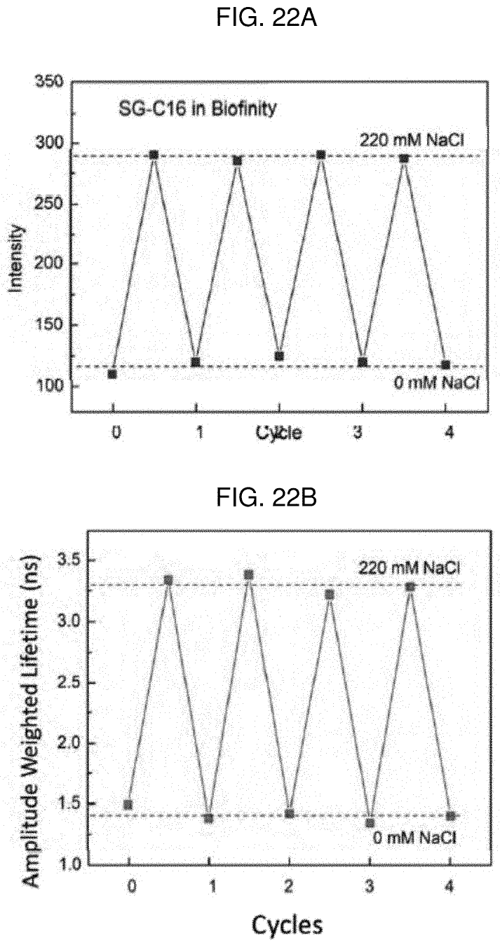

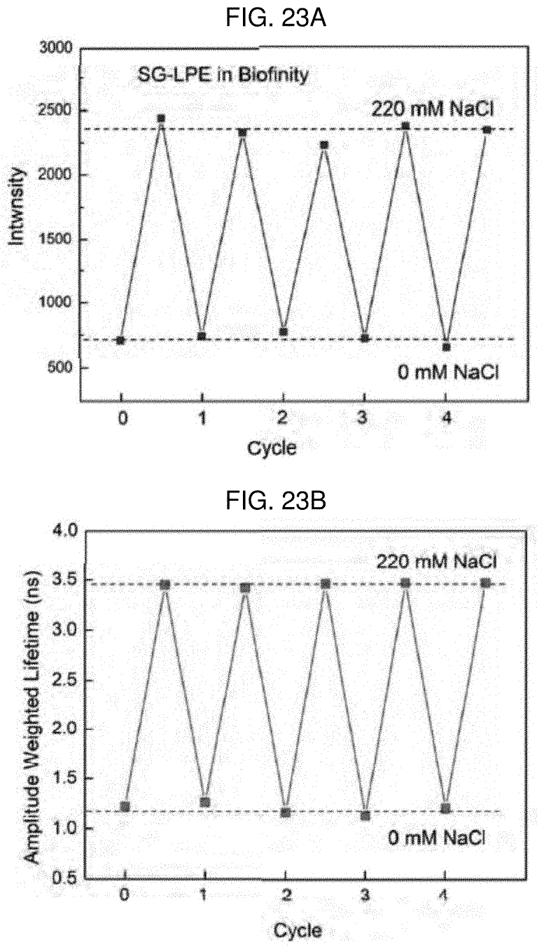

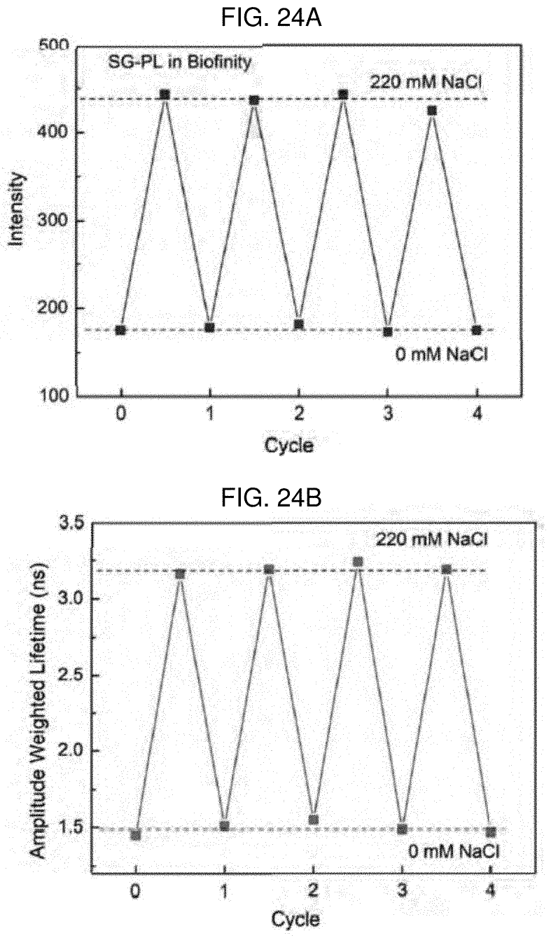

A clinically useful sodium contact lens must display a reversible response to sodium. To test this, the lenses were washed several times in 3 ml of probe-free buffer for each cycle. The constant fluorescence intensities demonstrate that SG-C16 is not washed out of the lenses. The sodium-dependent lifetime and intensity changes for SG-C16 lenses were completely reversible for concentration changes from 0 to 220 mM NaCl (FIG. 22). This figure shows the reversibility of a SG-C16-labeled Biofinity™ contact lens measured by intensity (FIG. 22A) and lifetime (FIG. 22B) with repeated cycling between no sodium and 220 mM NaCl. Measurements were performed on the center area of the lens using FLIM instrumentation. Similar reversibility and absence of probe washout were observed for SG-LPE and SG-PL (see FIG. 23 and FIG. 24, which present the same data as FIG. 22).

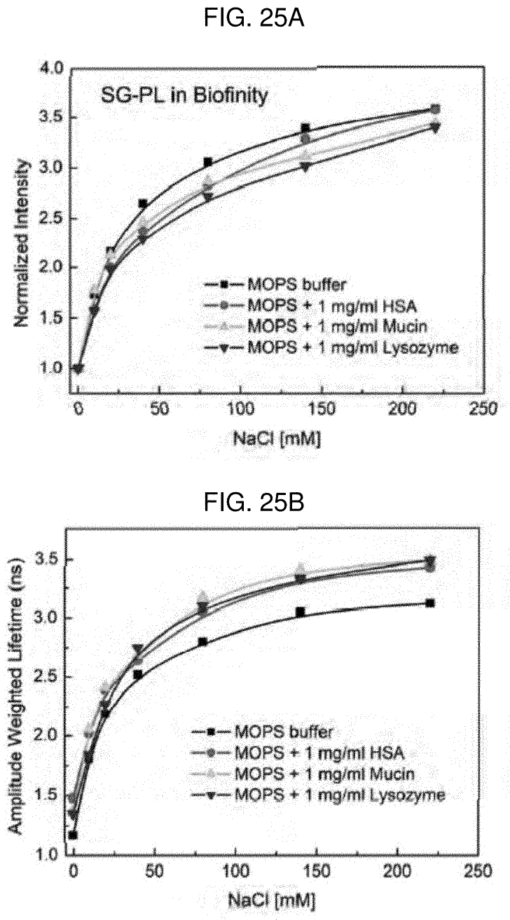

Tears contain a large number of proteins and water-soluble glycoproteins that could affect the sodium response. It was not practical to test all proteins and components in tears, so three different proteins were selected for testing. Lysozyme was selected because it is the most abundant (comprising 20-40% of tear proteins). Lysozyme binds quickly to many contact lenses, which is believed to be due to its net positive charge at neutral pH and the negative charges on most lenses. Serum albumin is present in tears at low concentrations but can increase under some conditions, so this protein was also selected. We also tested mucin type II (MUC2) which is 80% oligosaccharide by weight and is present in tears in a freely diffusible form. The sodium responses of SG-PL were completely unchanged in the presence of 1 mg/ml of each of these proteins. See results in FIG. 25, which shows the sodium-dependent intensity (FIG. 25A and lifetime responses (FIG. 25B) of SG-PL in Biofinity™ lenses in the absence and presence of the HSA, mucin or lysozyme. Measurements were performed on the center area of the lens using FLIM instrumentation.

### Example 9. Selection of Contact Lenses

Two commercially available contact lenses were selected for testing, Biofinity™ lenses and MyDay™ lenses, from Cooper Vision. Both lenses are based on silicone hydrogel (SiHG) polymers which have replaced a large fraction of the previous contact lenses made with HEMA-type hydrogels. The Biofinity™ and MyDay™ lenses appear to have different chemical and physical properties. Biofinity™ lenses are based on the polymer Comfilcon A, are approved for extended wear up to 30 days, and do not need to be removed while sleeping, but the current recommendations are for daily removal and cleaning. See FIG. 8A and FIG. 8B for illustrations of the lens material.

Extended wear is possible because SiHG lenses are highly permeable to oxygen. The Dk values of 128 for Biofinity™ lenses is higher than many SiHG lenses (see Table 11, below) and even higher than an equivalent thickness of water, which is near 80 Dk units. The high Dk values for the Biofinity™ lenses are due to silicone-rich regions which are highly permeable to oxygen. The water content is 48% and the total silicone volume is thought to be about 30%.

TABLE 11
Contact Lens Properties

| | | | Water | | Modulus |
|---|---|---|---|---|---|
| Polymer | Trade Name | Manufacturer | (%) | Dk | (MP) |
| Comfilcon | Biofinity ™ | Cooper Vision | 48 | 128 | 0.87 |
| Stenfilcon | MyDay ™ | Cooper Vision | 54 | 80 | 0.40 |

Even with this high silicone content, the Biofinity™ lenses remain highly permeable to water and ions in tears. This permeability is due to the presence of a semi-interpenetrating polymer network (IPN) with continuous channels which are essentially pure water or tear fluid, from the front to back surfaces of the lens (see FIG. 8). Since the lenses are optically clear the water channels must be smaller than visible wavelengths of light and are thought to be about 50 nm in diameter. The macromolecular structure shown in FIG. 8 suggests the presence of low polarity and non-polar to polar interface regions which have been demonstrated in recent papers. In addition, the Biofinity™ lenses bound pH and glucose-sensitivity fluorophores which contained hydrophobic side chains to localize the sensing fluorophores at the interface.

The MyDay™ lenses based on Stenfilcon A. These lenses were developed more recently, but are quickly becoming one of the most frequently prescribed lenses. MyDay™ lenses are prescribed for one-day use and contain a very low silicone content of 4.4% (Table 10, above), much less than the Biofinity™ lenses. Even with this low silicone content, the MyDay™ lenses still have high Dk values near 80. The high Dk value is claimed to be the result of “Smart Silicone Technology” which results in continuous silicone channels from the front to the back of the lenses. The IPN network in MyDay™ lenses is thought to be the opposite of the Biofinity™ lenses. See FIG. 8. The non-silicone region of the MyDay™ lenses is probably a non-silicone HG. The MyDay™ lenses are expected to have interference regions for binding of hydrophobic ISF. Binding of SG-PL was attempted because PL is frequently used to coat hydrophobic surfaces and hydrogels are typically negatively charged. The MyDay™ lenses were expected to be superior to Biofinity™ because of their higher flexibility and higher water content. However, the lenses are nearly identical in terms of patient comfort and corneal health.

### Example 10. Sodium-Responses of SG-PL in Lenses

For clinical use the labeled lenses must display spectral changes at sodium concentration present in lenses, which is near 120 mM. The parent fluorophore, Sodium Green itself, has a binding affinity near 5 mM. At 100 mM Na+ concentration the probe will be 95% in the sodium-bound form (equation 6) and essentially non-responsive to Na+ at higher concentrations. This consideration caused us to consider other Na+ probes or synthesizing a modified form of SG. The SG Na+ binding affinity can be decreased by changing the di-azacrown with mono-azacrown ether or chromium ether but that would allow the presence of only one dichloro-fluorescein per sensing molecule. In one embodiment, the sodium affinity of SG-PL may be lower in the lenses because binding to PL neutralizes the two carboxyl groups on the parent molecule SG (see Table 11, below) and these groups may contribute to the sodium binding affinity. Additionally, the positive charges of the large PL molecule may provide a repulsive effect on Na+ and decrease in the affinity of SG-PL. And finally, the SG portion of SG-PL may become partially buried in the non-polar regions of the lenses which could decrease the affinity.

TABLE 11
Chemical Structures of SPQ-C18 and the polarity-sensing probes 1,8-ANS
and Prodan ™.

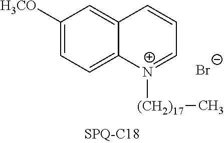

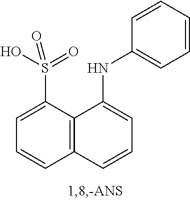 [SMILES: O=S(=O)(O)c1cccc2cccc(Nc3ccccc3)c12]

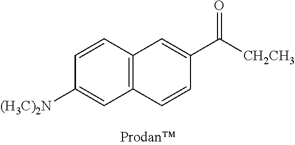

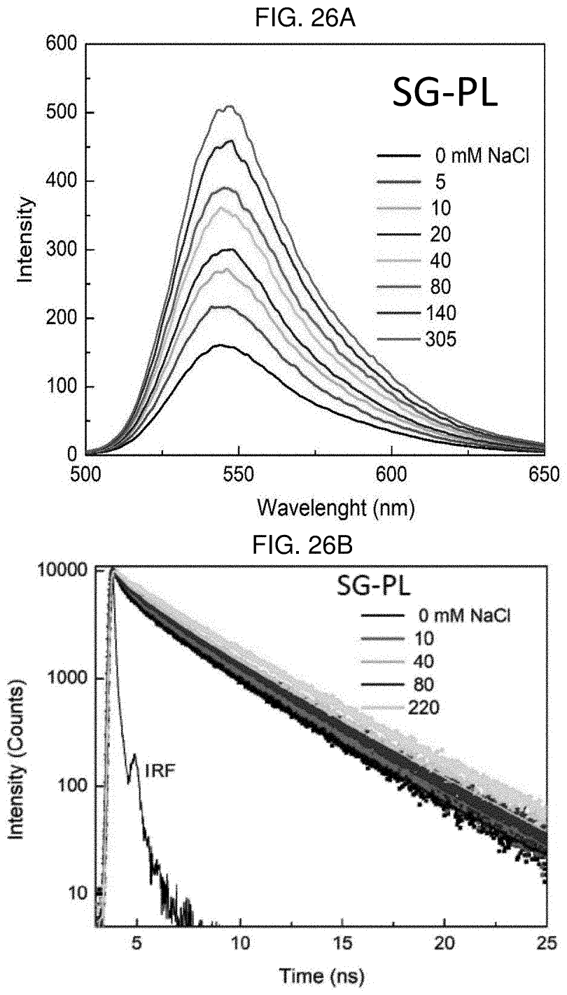

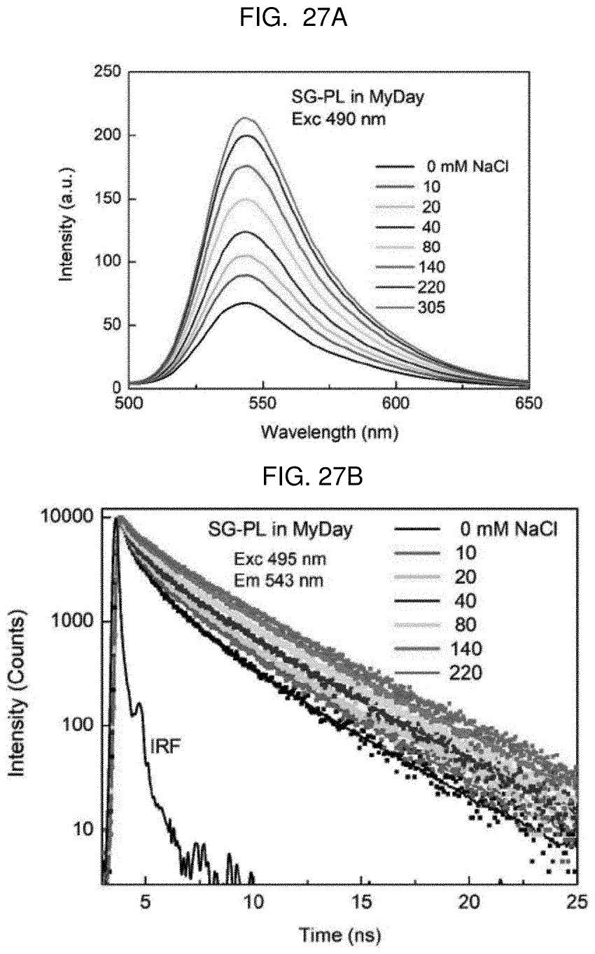

FIG. 26 presents sodium-dependent emission spectra (FIG. 26A) and intensity decays (FIG. 26B) of SG-PL in Biofinity™ contact lenses; FIG. 27 presents sodium-dependent emission spectra (FIG. 27A) and intensity decays (FIG. 27B) of SG-PL in MyDay™ contact lenses. The SG-PL emission spectra and intensity decay in Biofinity™ and MyDay™ lenses are shown in FIG. 26 and FIG. 27, respectively. The intensities increase about 2-fold upon addition of NaCl. The intensity decays become less rapid at higher concentrations of NaCl. The SG-PL intensity decays are strongly multi or non-exponential in the absence of Na+ and appear to be less heterogeneous at high Na+ concentrations. Results of the multi-exponential analyses are shown in second provisional, Tables 12 and 13, below. The intensities increase over 2-fold upon addition of NaCl to the surrounding solution. The intensity increased, the intensity decays became less rapid, the lifetimes became longer at higher Na⁺ concentrations. The SG-PL intensity decays are strongly multi or non-exponential in the absence of Na⁺ and appear to be less heterogeneous at high Na⁺ concentrations.

TABLE 12
Sodium-dependent intensity decays of SG-PL in Biofinity ™ lens, 20 mM MOPS,
pH 7.3.

| NaCl | τ₁ | τ₂ | τ₃ | | | | | | | τ_{α} | τ_{f} |
|---|---|---|---|---|---|---|---|---|---|---|---|
| (mM) | (ns) | (ns) | (ns) | α₁ | α₂ | α₃ | f₁ | f₂ | f₃ | (ns) | (ns) |
| 0 | 0.24 | 1.12 | 3.60 | 0.473 | 0.267 | 0.260 | 0.083 | 0.221 | 0.696 | 1.35 | 2.77 |
| 5 | 0.47 | 1.86 | 3.85 | 0.417 | 0.221 | 0.363 | 0.097 | 0.205 | 0.697 | 2.00 | 3.11 |
| 10 | 0.44 | 1.62 | 3.86 | 0.381 | 0.209 | 0.410 | 0.080 | 0.162 | 0.758 | 2.09 | 3.22 |
| 20 | 0.69 | 2.74 | 4.49 | 0.387 | 0.414 | 0.199 | 0.116 | 0.495 | 0.390 | 2.30 | 3.19 |
| 40 | 0.38 | 1.54 | 4.02 | 0.239 | 0.248 | 0.514 | 0.036 | 0.151 | 0.814 | 2.54 | 3.51 |
| 80 | 0.69 | 2.17 | 3.99 | 0.262 | 0.232 | 0.506 | 0.067 | 0.186 | 0.747 | 2.70 | 3.43 |
| 140 | 0.47 | 1.75 | 4.21 | 0.200 | 0.198 | 0.602 | 0.032 | 0.117 | 0.852 | 2.98 | 3.81 |
| 220 | 0.40 | 1.86 | 4.23 | 0.149 | 0.241 | 0.610 | 0.019 | 0.145 | 0.836 | 3.09 | 3.81 |

TABLE 13
Sodium-dependent intensity decays of SG-PL in MyDay, 20 mM MOPS, pH 7.3.

| NaCl | τ₁ | τ₂ | τ₃ | | | | | | | τ_{α} | τ_{f} |
|---|---|---|---|---|---|---|---|---|---|---|---|
| (mM) | (ns) | (ns) | (ns) | α₁ | α₂ | α₃ | f₁ | f₂ | f₃ | (ns) | (ns) |
| 0 | 0.23 | 1.17 | 3.41 | 0.477 | 0.291 | 0.231 | 0.090 | 0.274 | 0.636 | 1.24 | 2.51 |
| 10 | 0.26 | 1.29 | 3.54 | 0.376 | 0.317 | 0.307 | 0.062 | 0.257 | 0.681 | 1.59 | 2.76 |
| 20 | 0.27 | 1.35 | 3.61 | 0.416 | 0.313 | 0.271 | 0.075 | 0.278 | 0.646 | 1.51 | 2.73 |
| 40 | 0.27 | 1.33 | 3.60 | 0.362 | 0.300 | 0.339 | 0.057 | 0.231 | 0.711 | 1.72 | 2.89 |
| 80 | 0.34 | 1.50 | 3.72 | 0.339 | 0.289 | 0.372 | 0.059 | 0.224 | 0.717 | 1.93 | 3.02 |
| 140 | 0.35 | 1.48 | 3.72 | 0.275 | 0.273 | 0.452 | 0.044 | 0.185 | 0.770 | 2.18 | 3.15 |
| 220 | 0.34 | 1.55 | 3.75 | 0.260 | 0.282 | 0.457 | 0.039 | 0.195 | 0.765 | 2.24 | 3.19 |
| 305 | 0.36 | 1.61 | 3.81 | 0.242 | 0.267 | 0.491 | 0.037 | 0.180 | 0.783 | 2.39 | 3.29 |
| 405 | 0.56 | 2.07 | 3.86 | 0.242 | 0.316 | 0.442 | 0.054 | 0.263 | 0.683 | 2.50 | 3.21 |

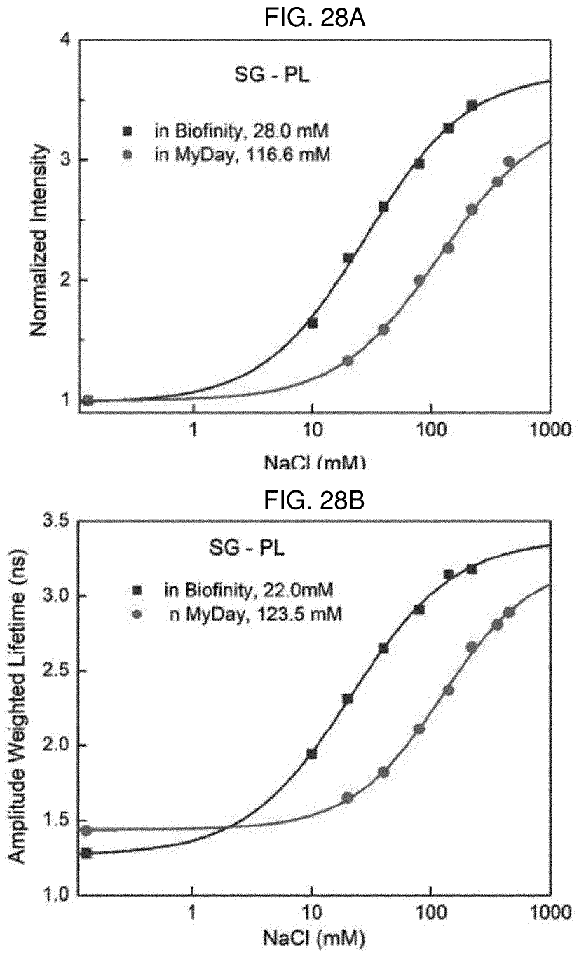

FIG. 28 presents binding curves (log scale) of sodium binding to SG-PL in Biofinity™ and MyDay™ lenses as measured by intensities (FIG. 28A) or lifetimes (FIG. 28B). Numerical values indicate the mid-points of the sodium response. The sodium binding curves (see FIG. 28) showed very different Na+ affinities for SG-PL in Biofinity™ as compared to MyDay™ lenses. In the Biofinity™ lenses, sodium binding is too strong to respond at 120 mM Na⁺ concentrations, but SG-PL in MyDay™ lenses displayed spectral changes up to 150 mM Na+ and are therefore suitable for measurement of Na⁺ concentrations in tears.

In some embodiments, weaker Na+ binding in MyDay™ as compared to Biofinity™ lenses can be caused by sodium concentrations around the fluorophore that are different in the two lenses. Furthermore, the Na⁺ concentrations near the fluorophore in Biofinity™ lenses could be the same as the bulk solution because the channels are thought to be pure water or tear fluid. In the MyDay™ lenses, the silicone channels may be surrounded by a hydrogel which occupies about one-half of the volume around the fluorophore, which decreases the effective Na⁺ concentration near the fluorophore, which in turn requires higher bulk-phase Na+ concentrations for binding.

### Example 11. Reversibility and Protein Interference in MyDay™ Lenses

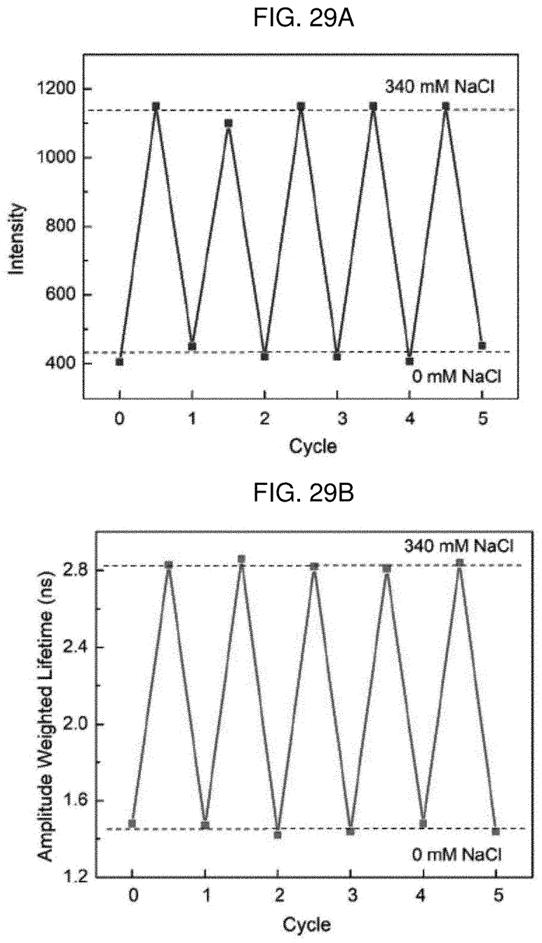

For clinical use the spectral changes must be reversible for increases and decreases in Na+ concentrations. Here, we have showed that the response of SG-PL was reversible and the probe did not wash out of the Biofinity™ lenses. The MyDay™ lenses also were tested for reversibility by cycling between 0 and 320 mM NaCl. During each cycle, the lenses were rinsed several times in 3 ml of buffer then placed in buffers with and without NaCl. The intensity and lifetime changes were completely reversible (FIG. 29). The reversible changes in fluorescence intensity (FIG. 29A) demonstrate that SG-PL is not washed out of the MyDay™ lenses.

Another important consideration in the effects of proteins or other tear components which could bind to the lenses and change the sodium response. Tears contain a large number of proteins and other biomolecules and it was not practical to test all of these. Three proteins were selected which are known to be present in tears; human tear lysozyme, human serum albumin and mucin type 2 (MUC2). For accuracy, we note that other mucins were detected in tears, but MUC2 was commercially available and somewhat water soluble.

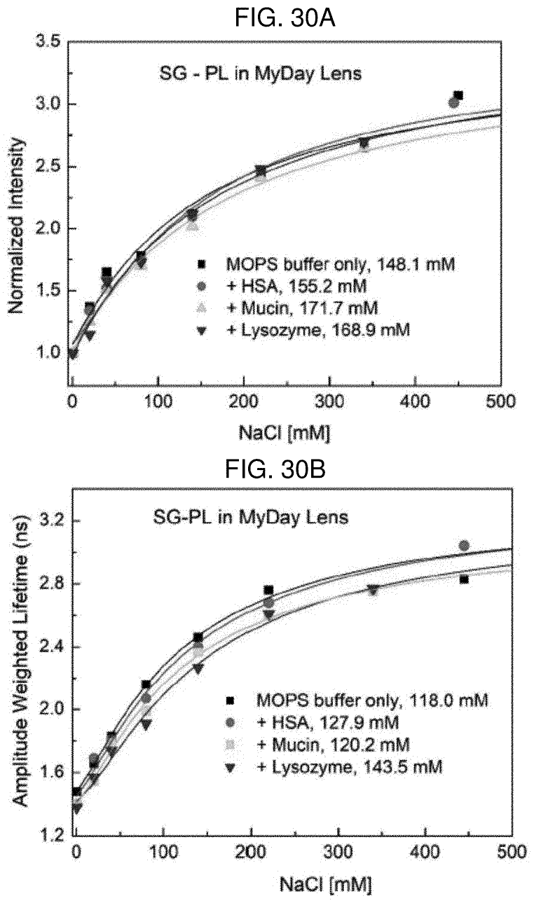

The response of SG-PL in MyDay™ lenses was not affected by the presence of 1 mg/ml of any of these three proteins. See FIG. 30. FIG. 30 shows the sodium responses of SG3-PL labeled MyDay™ lenses in the absence (MOPS buffer only) and presence of 1 mg/ml of HSA, Mucin and Lysozyme. Numerical values indicate the midpoints of respective curves. Similar results (no interference by the proteins) was found for SG-PL in Biofinity™ and MyDay™ lenses. The lenses were exposed to these proteins for about 2 hours, demonstrating that protein interference will not occur after about 2 hours contact with tear proteins.

### Example 12. Chloride-Sensitive Contact Lenses

Sodium sensing was accomplished by direct binding of Na⁺ to the fluorophore. Chloride sensing is accomplished by a different mechanism, collisional quenching. A large number of fluorophores are known which are by quenched by chloride ions, and there are typically quinolinium or acridinium derivatives. Collisional quenching occurs when a chloride-sensitive fluorophore undergoes diffusion-mediated contact with a chloride ion while the fluorophore is in the excited state. As a result, the amount of quenching is limited only by the highest possible concentrations of the quenchers. The sensitivity of the sensors depends on its fluorescent lifetime in the absence of quenching (τ₀). A longer lifetime provides more time for a molecular collision and more quenching at a given chloride concentration (equation 8). The unquenched lifetime τ₀ of the fluorophore must be long enough for quenching to occur, but not too long because then the emission will be too weak.

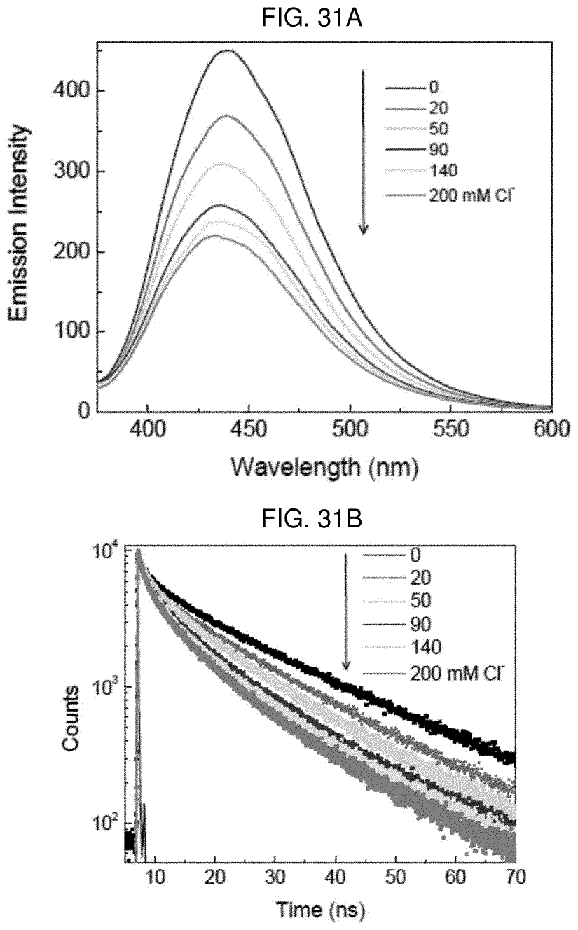

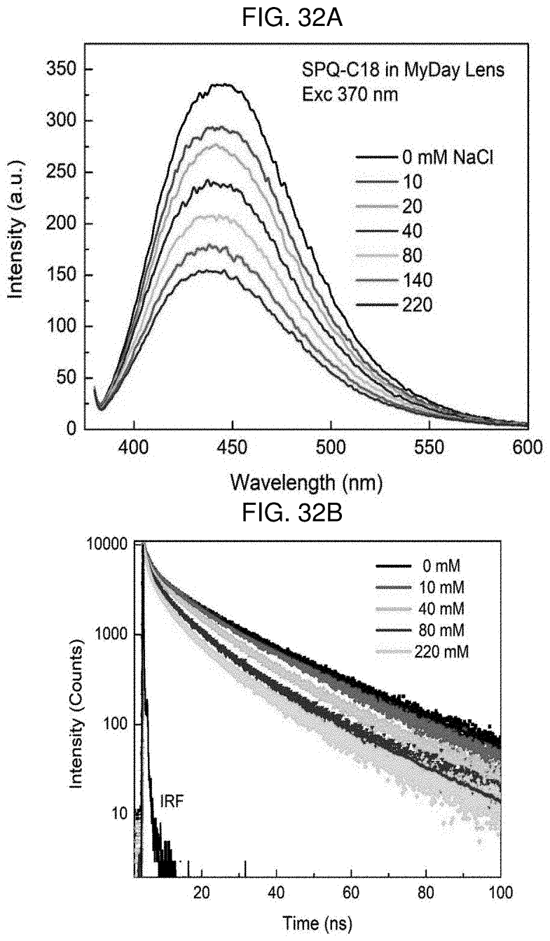

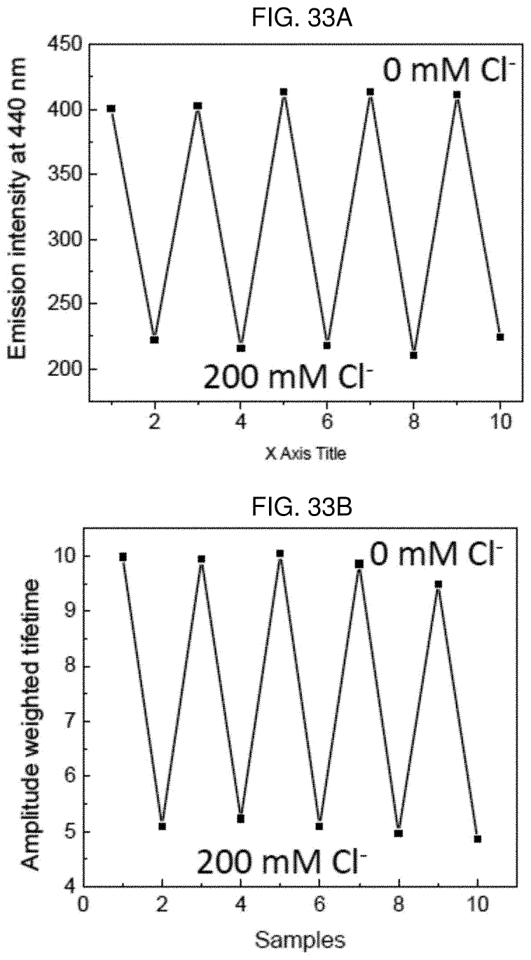

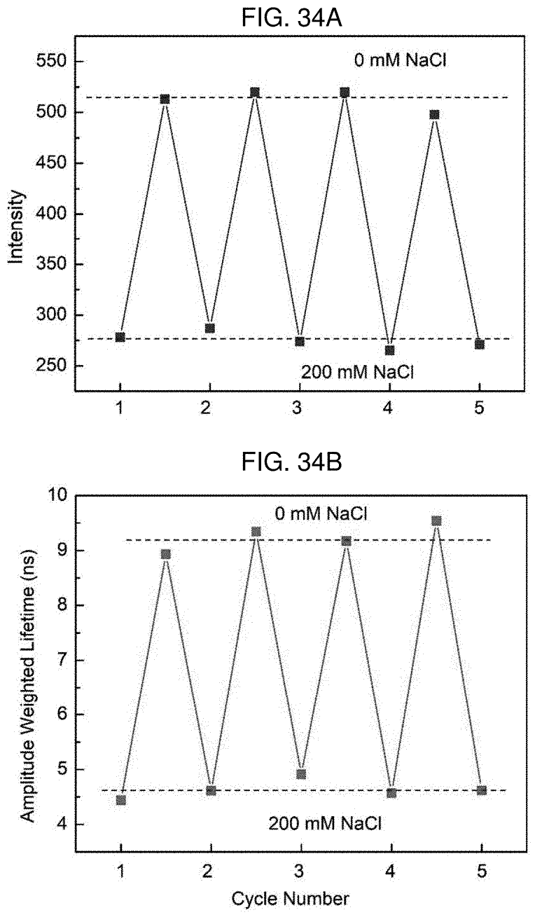

Chloride-sensitive lenses were prepared using SPQ-C18. The quinolinium moiety in SPQ-C18 can be used for chloride sensing in tears. Its unquenched lifetime near 18.5 ns (see Table 14 and Table 15, below) which results in significant changes up to chloride concentrations of 220 mM, which is well above the physiological range. Biofinity™ and MyDay™ lenses labeled with SPQ-C18 were both found to display significant quenching at physiological concentrations of chloride near 120 mM. The emission intensities of SPQ-C18 were quenched by about 50% at this chloride concentration (FIG. 31 and FIG. 32). The emission spectra are not changed by quenching so intensity-based sensing may be different in a realworld application. However, the rate of the intensity decays is greatly increased by chloride, so that lifetime-based sensing is the most promising approach here. Chloride quenching was completely reversible in both Biofinity™ and MyDay™ lenses (see FIG. 33 and FIG. 34 respectively).

Traditional intensities measurements could be difficult if the patient's eye is moving or blinking. For this reason, in some embodiments, a clinically useful chloride-sensitive lens contains a reference fluorophore which is not sensitive to chloride and displays emission in a different wavelength range. A more direct measurement of the chloride concentration can be obtained from measurements of the fluorescence lifetime which can be independent of total intensity or intensity fluctuations.

Chloride quenching was analyzed using the Stern-Volmer equation for intensities and lifetimes (equations 7 and 8). In this analysis, we used the average lifetimes (equation 2) that is more appropriate for quenching. These plots show that both the intensities and lifetimes continue to change up to 200 mM chloride. In contrast quenching of SP-PL, both intensity and lifetime, continue to decrease as the chloride concentration is increased. At the molecular level, the most interesting term is the biomolecular quenching constant k_{q}. This value can be understood by comparison with quenching of a fluorophore in solution without any steric barriers. For the water-soluble fluorophores SPQ-C3, values of 6=26 ns, K=140 M-1, and k=5.4×109 M-1 sec-1 were found.

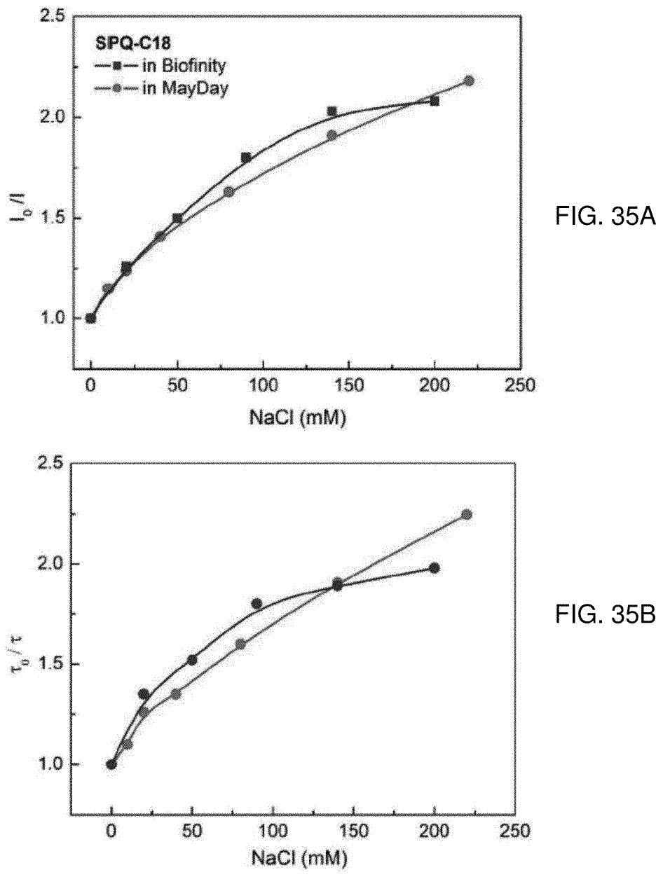

This approach for SPQ-C18 in lenses is shown in both intensities and amplitude-weighted lifetimes (FIG. 35A and FIG. 35B). These Stern-Volmer plots show that both the intensities and lifetimes continue to change up to 200 mM chloride. The similar quenching constants in both lenses suggests a similar local environment. This behavior is different from SG-PL where the binding site can be saturated at high Na⁺ concentrations. In contrast, quenching of OD-MQB, both intensity and lifetime, continue to decrease as the chloride concentration increased. The steady-state intensities and intensity decays can be used to calculate the bimolecular quenching constant k_{q}. This value can be interpreted by comparison with quenching of a fluorophore in solution without any steric barriers. For this measurement the water-soluble fluorophore 1-propenyl-6-methoxyquinolinium bromide (SPQ-C3) was used. This is the same structure of SPQ-C18 but with a shorter, three carbon propenyl chain. The unquenched lifetime (τ₀) of P-MQB was 26 ns, and the Stern-Volmer constant K was 140 M⁻¹. From these values, we calculated the k_{q} of 5.4×10⁹ M⁻¹ sec⁻¹ which is a typical value for efficient quenching.

Using the lifetime measurement (see Tables 14 and 15, below) values of 8.12 M-1 and 7.44-1 M-1 for SPQ-C18 in the Biofinity™ and MyDay™ lenses, respectively, were found. Using the unquenched lifetimes the respective values of k are 0.41×10⁹ and 0.37×10⁹ m-1 sec-1. These values show that quenching is reduced by about a factor of 10 for lens-bound fluorophores. This unexpected change allows these fluorophores to be effective for tear electrolyte detection.

The observed limit of detection and limit of quantification of chloride ion with SPQ-C18 labeled lens are 10 and 25 mM, respectively. The reduced quenching observed for SPQ-C18 in lens is a favorable result because the intensities and lifetimes are sensitive to chloride while the SPQ-C18 signal is not quenched too strongly. Importantly, the quenching constants are nearly the same in both lenses, which supports the conjecture that the sensing portion of SPQ-C18 is located in a similar aqueous phase. The 10-fold reduction in k_{q} from SPQ-C18 can be explained by chloride diffusion from only the aqueous side of the interface, by partial shielding of the quinolinium moiety in the silicon phase or the presence of other molecules located at the water-silicon interface.

TABLE 14
Chloride-dependent intensity decay analysis of SPQ-C18 in Biofinity ™ contact
lenses at 0 nM and 100 mM chloride concentration (phosphate buffer, pH 7.2).

| NaCl | τ₁ | τ₂ | τ₃ | | | | | | | τ_{α} | τ_{f} |
|---|---|---|---|---|---|---|---|---|---|---|---|
| (mM) | (ns) | (ns) | (ns) | α₁ | α₂ | α₃ | f₁ | f₂ | f₃ | (ns) | (ns) |
| 0 | 0.69 | 5.15 | 20.74 | 0.36 | 0.21 | 0.44 | 0.02 | 0.10 | 0.88 | 10.35 | 18.66 |
| 20 | 0.65 | 4.98 | 17.88 | 0.41 | 0.21 | 0.38 | 0.03 | 0.13 | 0.84 | 8.08 | 15.65 |
| 50 | 0.73 | 5.54 | 16.81 | 0.38 | 0.29 | 0.33 | 0.03 | 0.22 | 0.75 | 7.38 | 13.76 |
| 90 | 0.75 | 5.33 | 16.41 | 0.43 | 0.31 | 0.26 | 0.05 | 0.26 | 0.68 | 6.18 | 12.49 |
| 140 | 0.82 | 5.43 | 16.26 | 0.44 | 0.33 | 0.22 | 0.06 | 0.31 | 0.62 | 5.81 | 11.91 |
| 200 | 0.77 | 4.92 | 15.93 | 0.48 | 0.34 | 0.20 | 0.08 | 0.34 | 0.58 | 4.90 | 11.03 |

TABLE 15
Chloride-dependent intensity decay analysis of SPQ-C18 in MyDay ™ contact
lenses at 0 nM and 100 mM chloride concentration (20 mM MOPS, pH 7.3).

| NaCl | τ₁ | τ₂ | τ₃ | | | | | | | τ_{α} | τ_{f} |
|---|---|---|---|---|---|---|---|---|---|---|---|
| (mM) | (ns) | (ns) | (ns) | α₁ | α₂ | α₃ | f₁ | f₂ | f₃ | (ns) | (ns) |
| 0 | 1.03 | 8.00 | 22.78 | 0.560 | 0.187 | 0.253 | 0.074 | 0.191 | 0.735 | 7.83 | 18.36 |
| 10 | 1.05 | 7.22 | 18.80 | 0.496 | 0.290 | 0.214 | 0.078 | 0.315 | 0.607 | 6.63 | 13.76 |
| 20 | 1.12 | 7.04 | 19.59 | 0.554 | 0.263 | 0.183 | 0.102 | 0.306 | 0.592 | 6.05 | 13.86 |
| 40 | 1.06 | 6.78 | 18.24 | 0.590 | 0.275 | 0.136 | 0.126 | 0.375 | 0.499 | 4.96 | 11.78 |
| 80 | 1.05 | 5.58 | 17.70 | 0.601 | 0.284 | 0.115 | 0.148 | 0.373 | 0.478 | 4.25 | 10.71 |
| 140 | 1.01 | 5.39 | 17.05 | 0.649 | 0.253 | 0.099 | 0.177 | 0.368 | 0.455 | 3.70 | 9.92 |
| 220 | 0.94 | 4.71 | 14.81 | 0.636 | 0.282 | 0.081 | 0.191 | 0.425 | 0.284 | 3.13 | 7.87 |

### Example 13. Excitation and Emission Spectra

Excitation and emission spectra of 6HQ-C18 in Comfilcon A lens were obtained as follows. Emission and excitation spectra of the 6HQ-C18 labeled lenses fixed to a specially designed contact lens mount were measured using a Varian Cary Eclipse spectrofluorometer using xenon arc lamp as the excitation source.

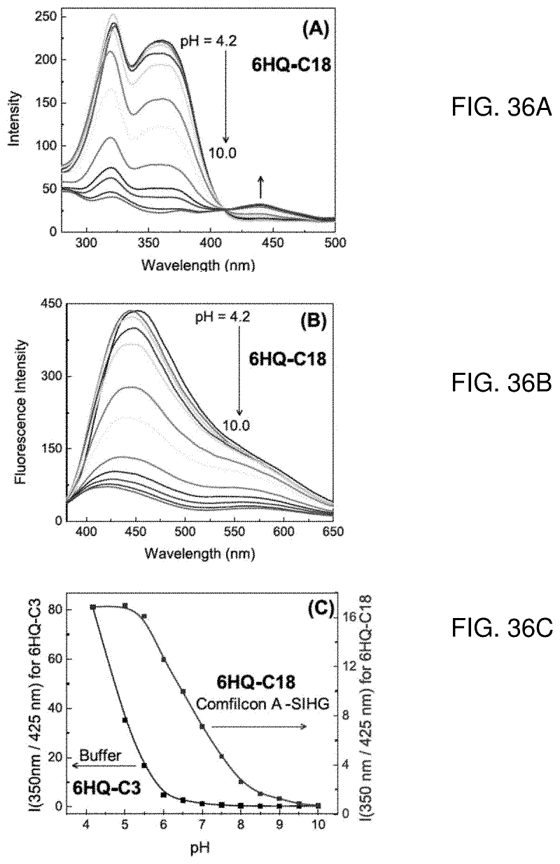

See FIG. 36A and FIG. 36B for the excitation (FIG. 36A) and emission (FIG. 36B) spectra of 6HQ-C18 in a Comfilcon A lens. Emission was monitored at 580 nm and λex=350 nm. The mid-point of the transition for 6HQ-C18 in the lens was near 6.5-7.0, which is 2 log units higher than observed for a water-soluble version of the same probe, 6HQ-C3 (See FIG. 36C). This is a favorable result because the pKa is shifted to a value closer to physiological pH. This shift in the pKa suggests that the 6HQ moiety is either partially buried in the silicone region of the lens or proton dissociation is restricted to some extent by the silicone-water interface. Thus, binding to the contact lens as described herein surprisingly allows one to use this fluorophore, even though the spectra measured in solution would indicate it would not be useful for measuring the analyte in tears.

### Example 14. Comparison of SPQ-C3 in Water and SPQ-C18 in a Stenfilcon a Contact Lens

Chloride quenching was observed for SPQ-C3 in water and for SPQ-C18 in a Stenfilcon A (Aspire™) contact lens. Emission spectra and time-dependent decays were obtained (data not shown). Stern-Volmer plots were obtained in water and in a Stenfilcon A (Aspire™) contact lens by plotting the normalized intensities with respect to the added chloride concentrations.

The bimolecular quenching constant for SPC-C3 in water was measured at 5.4×10⁹ M⁻¹ s⁻¹ (see FIG. 37). This value is consistent with the known diffusion constant of chloride in water with a quenching efficiency of 100%, which means every diffusive contact results in quenching. The Bimolecular quenching constant for SPQ-C18 bound to the contact lens was measured at 1×10⁹ M⁻¹ s⁻¹ (see FIG. 37), which is a 5.4 decrease in the collision rate of SPQ-C3 in water. This is a favorable result for an electrolyte contact lens because binding of SPQ-C18 to the SiHG lens results in a H-ISF which is most sensitive to chloride concentrations present in tears. These data show that addition of the hydrophobic moiety to the fluorophore and binding to the contact lens alters the quenching constant, allowing the probe to be used for detecting chloride at physiological concentrations.

### Example 15. Binding Affinity of Sodium to SG-PL

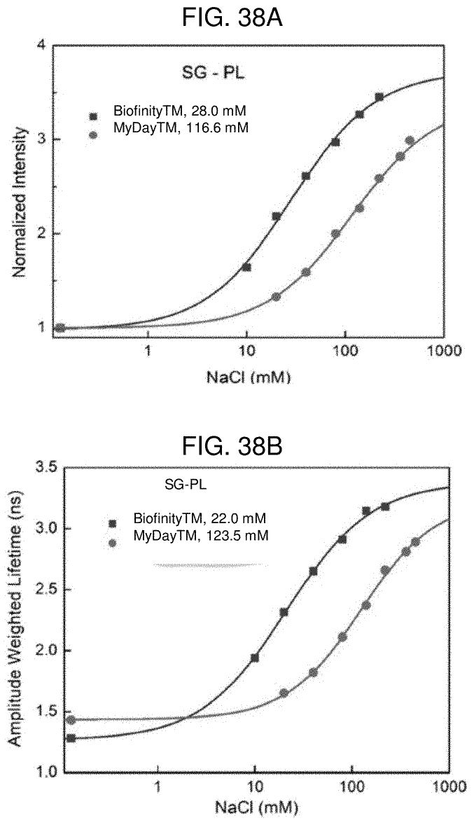

Binding affinity curves of SG-PL with sodium in Biofinity™ and MyDay™ lenses were produced by plotting the normalized intensities with respect to the added sodium concentrations. The curves were measured by intensities at peak maximum (FIG. 38A) or amplitude-weighted lifetimes (FIG. 38B). The numerical values indicate the mid-points of the sodium response.

The sodium binding curves revealed different Na⁺ affinities for SG-PL in Biofinity™ as compared to MyDay™ lenses. In the Biofinity™ lenses, sodium binding is close to saturation at 120 mM Na⁺, but SG-PL in MyDay™ lenses displayed spectral changes up to 150 mM Na⁺, and therefore are responsive across the physiological Na⁺ concentrations in tears. Without wishing to be bound by theory, one possible reason for this difference between MyDay™ and Biofinity™ lenses is the different sodium concentrations around the fluorophore in the two lenses. The Na⁺ concentrations near the fluorophore in Biofinity™ lenses could be similar to that in the bulk solution because the channels are thought to be pure water or tear fluid. In the MyDay™ lenses, the silicone channels may be surrounded by other hydrogels which occupy about one-half of the volume around the fluorophore. These polymers could decrease the effective Na⁺ concentration near the fluorophore. Overcoming this displacement effect would then require higher bulk phase Na⁺ concentrations for binding.

### Example 16. Analysis of SG Sodium Fluorophore Probes

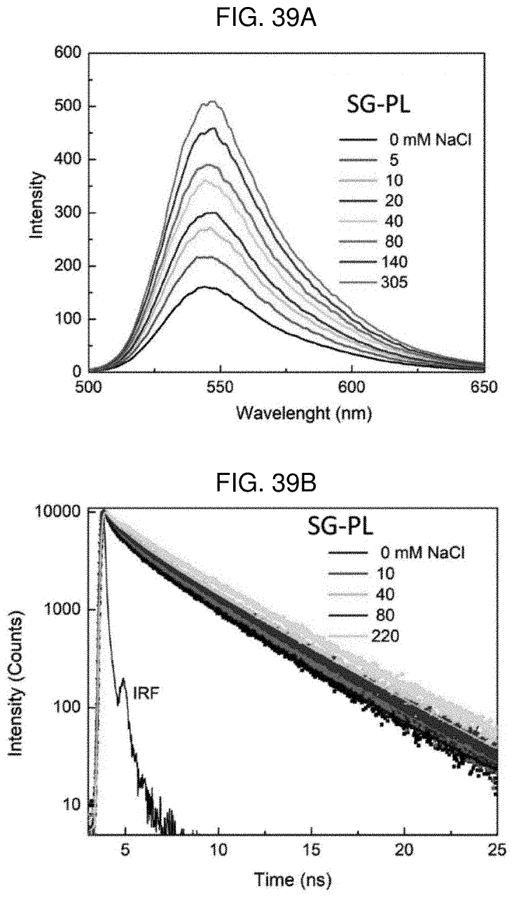

Multi-exponential analyses are summarized in Tables 16-19, below. SG in buffer displayed a binding affinity near 6.5 mM, which is too high for measurements of sodium in tears (see FIG. 39). For FIG. 39, lifetime measurements were performed on FluoTime 300 instrument. Numerical values correspond to midpoints of sodium-dependent responses.

The sodium affinity was decreased to values near 30 mM for the three probes (SG-C16, SG-PLE, SG-PL) bound to Biofinity™ lenses. Changes in intensity and lifetime continue through the physiological sodium range in tears of 120 mM, but the changes are smaller above 150 mM sodium. We expect the sodium probes for the final electrolyte contact lens will require slightly weaker sodium binding. This can be accomplished by modification of the sodium binding structure by the use of crown ethers with no nitrogen atoms or mono-azo crown ethers. Additionally, the sodium affinity may be dependent on the type of contact lens polymer. The essential point is that sodium concentrations can be determined with labeled contact lenses and the sodium binding affinities can be adjusted to match the physiological sodium concentration in tears.

TABLE 16
Intensity decays of SG-C16 in Biofinity lenses, 20 mM MOPS, pH 7.3

| NaCl | τ₁ | τ₂ | τ₃ | | | | | | | τ_{α} | τ_{f} |
|---|---|---|---|---|---|---|---|---|---|---|---|
| (nM) | (ns) | (ns) | (ns) | α₁ | α₂ | α₃ | f₁ | f₂ | f₃ | (ns) | (ns) |
| 0 | 0.26 | 1.28 | 3.54 | 0.376 | 0.317 | 0.306 | 0.062 | 0.257 | 0.681 | 1.59 | 2.76 |
| 5 | 0.29 | 1.33 | 3.73 | 0.378 | 0.198 | 0.424 | 0.056 | 0.135 | 0.810 | 1.95 | 3.21 |
| 10 | 0.38 | 1.66 | 3.79 | 0.351 | 0.229 | 0.420 | 0.063 | 0.181 | 0.757 | 2.10 | 3.19 |
| 20 | 0.36 | 1.76 | 3.86 | 0.284 | 0.218 | 0.499 | 0.042 | 0.160 | 0.798 | 2.41 | 3.37 |
| 40 | 0.51 | 2.02 | 3.83 | 0.260 | 0.244 | 0.495 | 0.053 | 0.196 | 0.751 | 2.52 | 3.30 |
| 80 | 0.42 | 1.75 | 3.81 | 0.161 | 0.359 | 0.580 | 0.025 | 0.166 | 0.809 | 2.73 | 3.38 |
| 140 | 0.39 | 1.63 | 3.82 | 0.183 | 0.16.8 | 0.649 | 0.025 | 0.097 | 0.878 | 2.82 | 3.52 |

TABLE 17
Intensity decays of SG-LPE in Biofinity lenses, 20 mM MOPS, pH 7.3

| NaCl | τ₁ | τ₂ | τ₃ | | | | | | | τ_{α} | τ_{f} |
|---|---|---|---|---|---|---|---|---|---|---|---|
| (nM) | (ns) | (ns) | (ns) | α₁ | α₂ | α₃ | f₁ | f₂ | f₃ | (ns) | (ns) |
| 0 | 0.25 | 1.10 | 3.58 | 0.524 | 0.256 | 0.220 | 0.110 | 0.235 | 0.655 | 1.20 | 2.63 |
| 5 | 0.25 | 1.18 | 3.64 | 0.426 | 0.249 | 0.325 | 0.067 | 0.186 | 0.748 | 1.58 | 2.96 |
| 10 | 0.30 | 1.35 | 3.70 | 0.378 | 0.240 | 0.382 | 0.061 | 0.175 | 0.764 | 1.85 | 3.08 |
| 20 | 0.37 | 1.57 | 3.74 | 0.328 | 0.231 | 0.441 | 0.057 | 0.169 | 0.773 | 2.13 | 3.18 |
| 40 | 0.39 | 1.67 | 3.78 | 0.262 | 0.244 | 0.494 | 0.043 | 0.171 | 0.786 | 2.38 | 3.27 |
| 80 | 0.41 | 1.94 | 3.97 | 0.210 | 0.252 | 0.538 | 0.032 | 0.181 | 0.788 | 2.71 | 3.49 |
| 140 | 0.43 | 1.63 | 3.75 | 0.189 | 0.221 | 0.590 | 0.031 | 0.136 | 0.833 | 2.65 | 3.36 |

TABLE 18
Intensity decays of SG-PL in Biofinity ™ lenses, 20 mM MOPS, pH 7.3

| NaCl | τ₁ | τ₂ | τ₃ | | | | | | | τ_{α} | τ_{f} |
|---|---|---|---|---|---|---|---|---|---|---|---|
| (mM) | (ns) | (ns) | (ns) | α₁ | α₂ | α₃ | f₁ | f₂ | f₃ | (ns) | (ns) |
| 0 | 0.24 | 1.12 | 3.60 | 0.473 | 0.267 | 0.260 | 0.083 | 0.221 | 0.696 | 1.35 | 2.77 |
| 5 | 0.47 | 1.86 | 3.85 | 0.417 | 0.221 | 0.363 | 0.097 | 0.205 | 0.697 | 2.00 | 3.11 |
| 10 | 0.44 | 1.62 | 3.86 | 0.381 | 0.209 | 0.410 | 0.080 | 0.162 | 0.758 | 2.09 | 3.22 |
| 20 | 0.69 | 2.74 | 4.49 | 0.387 | 0.414 | 0.199 | 0.116 | 0.495 | 0.390 | 2.30 | 3.19 |
| 40 | 0.38 | 1.54 | 4.02 | 0.239 | 0.248 | 0.514 | 0.036 | 0.151 | 0.814 | 2.54 | 3.51 |
| 80 | 0.69 | 2.17 | 3.99 | 0.262 | 0.232 | 0.506 | 0.067 | 0.186 | 0.747 | 2.70 | 3.43 |
| 140 | 0.47 | 1.75 | 4.21 | 0.200 | 0.198 | 0.602 | 0.032 | 0.117 | 0.852 | 2.98 | 3.81 |
| 220 | 0.40 | 1.86 | 4.23 | 0.149 | 0.241 | 0.610 | 0.019 | 0.145 | 0.836 | 3.09 | 3.81 |

TABLE 19
Intensity decays of SG in 20 mM MOPS buffer 8 mM KCl

| NaCl | τ₁ | τ₂ | τ₃ | | | | | | | τ_{α} | τ_{f} |
|---|---|---|---|---|---|---|---|---|---|---|---|
| (mM) | (ns) | (ns) | (ns) | α₁ | α₂ | α₃ | f₁ | f₂ | f₃ | (ns) | (ns) |
| 0 | 0.36 | 1.03 | 3.59 | 0.703 | 0.155 | 0.142 | 0.272 | 0.174 | 0.554 | 0.92 | 2.27 |
| 5 | 0.39 | 1.44 | 3.28 | 0.439 | 0.244 | 0.317 | 0.111 | 0.223 | 0.666 | 1.57 | 2.55 |
| 10 | 0.40 | 1.43 | 3.22 | 0.341 | 0.276 | 0.383 | 0.078 | 0.224 | 0.699 | 1.77 | 2.60 |
| 20 | 0.57 | 2.00 | 3.39 | 0.324 | 0.353 | 0.323 | 0.094 | 0.355 | 0.551 | 1.99 | 2.63 |
| 30 | 0.54 | 1.72 | 3.27 | 0.255 | 0.349 | 0.396 | 0.068 | 0.295 | 0.638 | 2.03 | 2.63 |
| 50 | 0.66 | 1.95 | 3.35 | 0.259 | 0.380 | 0.361 | 0.081 | 0.349 | 0.570 | 2.12 | 2.64 |
| 90 | 0.64 | 1.93 | 3.36 | 0.242 | 0.416 | 0.342 | 0.074 | 0.380 | 0.545 | 2.11 | 2.61 |
| 150 | 0.81 | 2.48 | 4.38 | 0.305 | 0.605 | 0.091 | 0.116 | 0.699 | 0.185 | 2.15 | 2.64 |
| 210 | 0.70 | 1.88 | 3.29 | 0.224 | 0.424 | 0.352 | 0.074 | 0.377 | 0.549 | 2.12 | 2.57 |

### Example 17. Spotting Fluorophore Probes for Multiplex Lenses

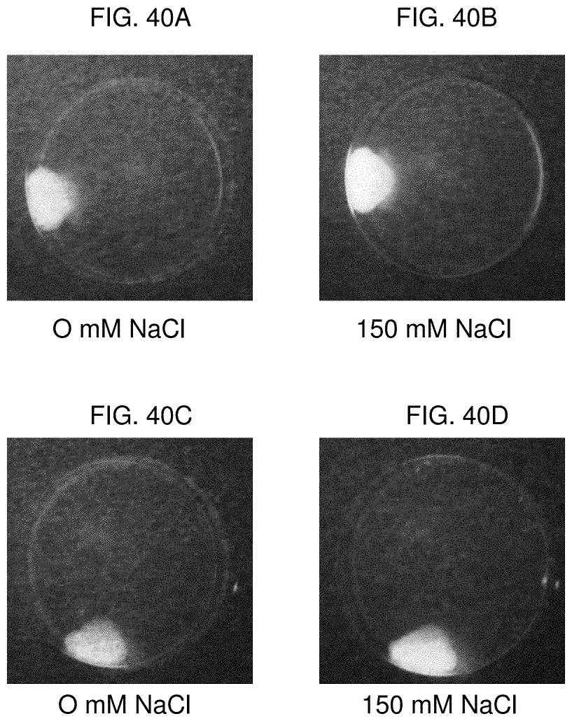

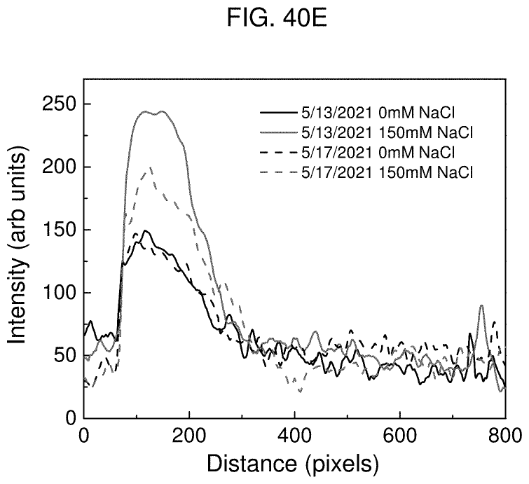

SG3 was applied to a discrete area on the edge of Biofinity™ lenses in pH 7.3 MOPS buffer with 0 mM NaCl or 150 mM NaCl as indicated in FIG. 40A-FIG. 40D. λex=dispersed 473 nm laser, with 500 nm LP before camera. FIG. 40E shows an intensity line tracing along the dotted lines shown in the photographs using ImageJ™. These data show that the probes do not diffuse laterally so that multiplex lenses can be made by spotting the different fluorophore probes on discrete areas or spots on the contact lens.

### Example 18. Single Contact Lens Sensitive to Both Sodium and Chloride

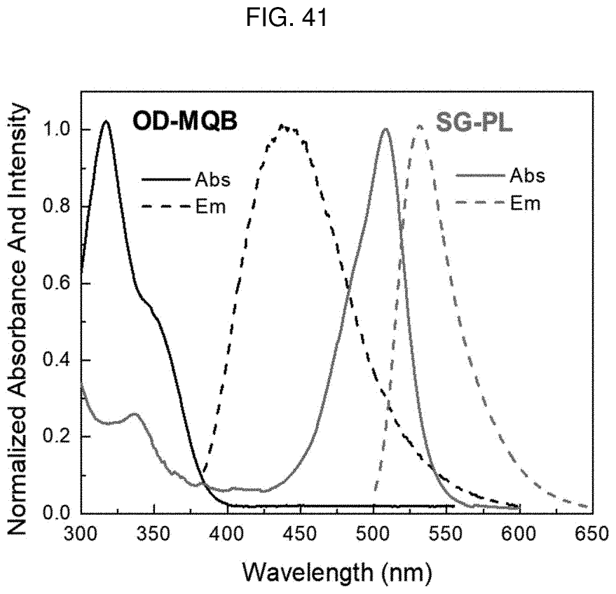

SG-PL and SPQ-C18 have widely spaced absorption and emission spectra (see FIG. 41). Emission spectra are in Biofinity™ lenses. This allows one to use both probes in a single contact lens easily, to provide lens sensitive to both Na⁺ and Cl⁻, with or without physical separation of the probes in the lens in discrete regions of the lens. The absorption and emission spectra show that SPQ-C18 can be excited near 360 nm with the emission observed at 454 nm. SG-PL will also absorb light near 360 nm but its emission at 550 nm will not overlap or interfere with measurement of SPQ-C18 emission. SG-PL can be excited at 490 nm where SPQ-C18 does not absorb and will not interfere with the SG-PL measurements.

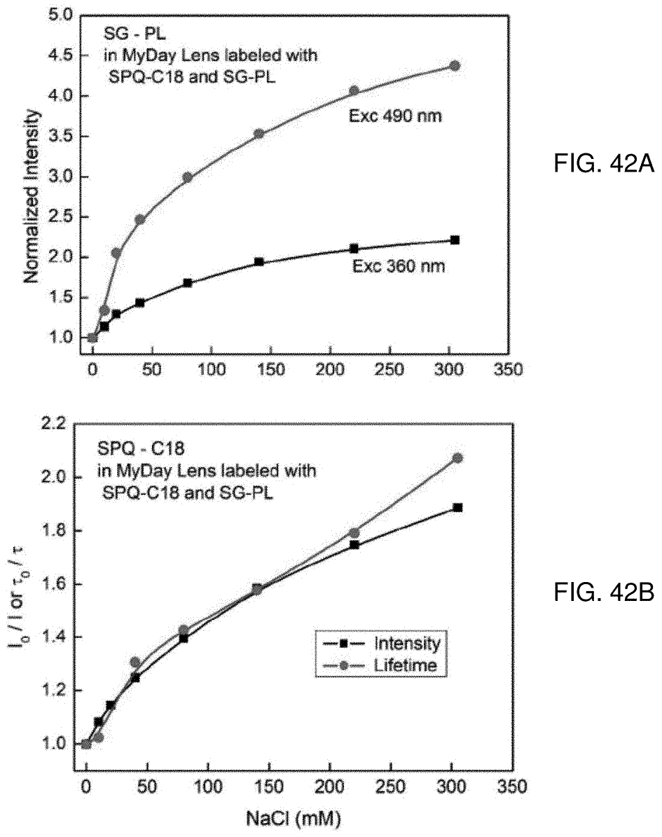

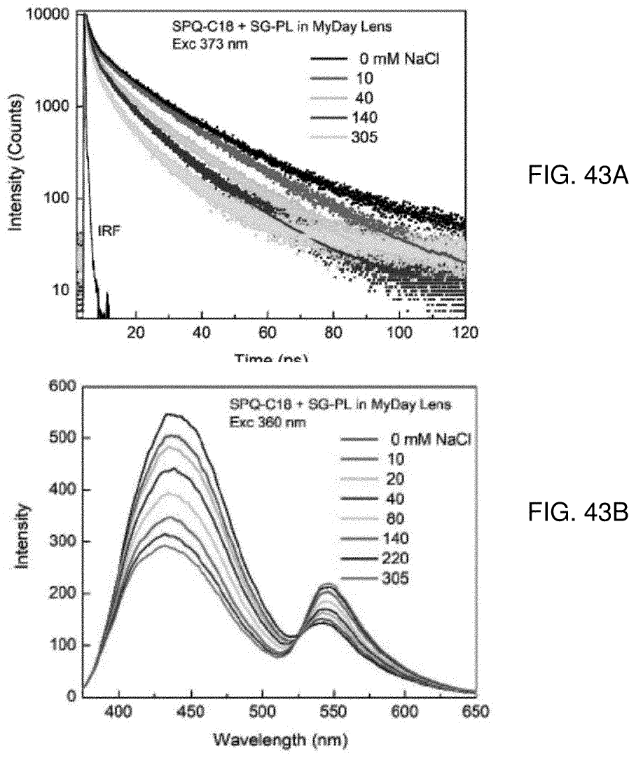

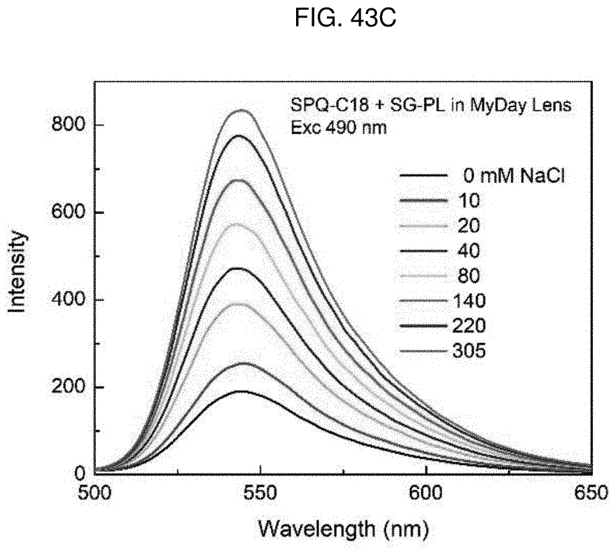

MyDay™ lenses were labeled with both SG-PL and SPQ-C18 using both probes in aqueous methanolic solution (50:50 v/v) instead of single probe. The labeled lenses were washed with deionized water prior to use in the analyte response studies. The sodium and chloride responses were measured for the doubly labeled MyDay™ lens. Surprisingly, the response to chloride (see FIG. 42 and FIG. 43) in the double-labeled lens was essentially identical to the response of the single-labeled lens (labeled only with SPQ-C18). According to accepted theory, it was possible for fluorescence resonance energy transfer to occur from SPQ-C18 to SG-PL in the small silicone volume of the MyDay™ lenses when both probes are in close proximity. Such an effect would decrease the unquenched lifetime of SPQ-C18, change the calibration curve, and decrease the sensitivity to chloride. Additionally, the sodium response of SG-PL remained the same in the presence of SPQ-C18, showing that the presence of SPQ-C18 in the lens does not alter sodium response. These results demonstrate a contact lens to detect both sodium and chloride can be made with or without physical separation of the probes into different regions of the contact lens.

### Example 19. Binding of Polarity Sensitive Probes to MyDay™ Lenses

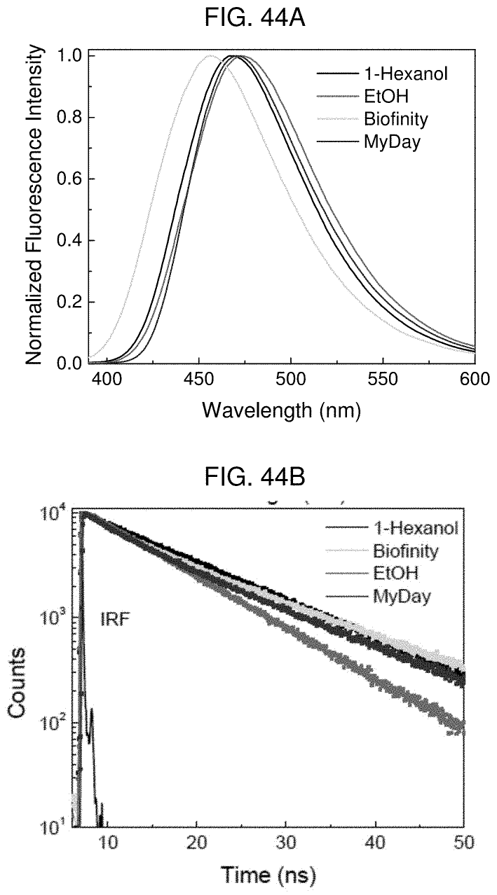

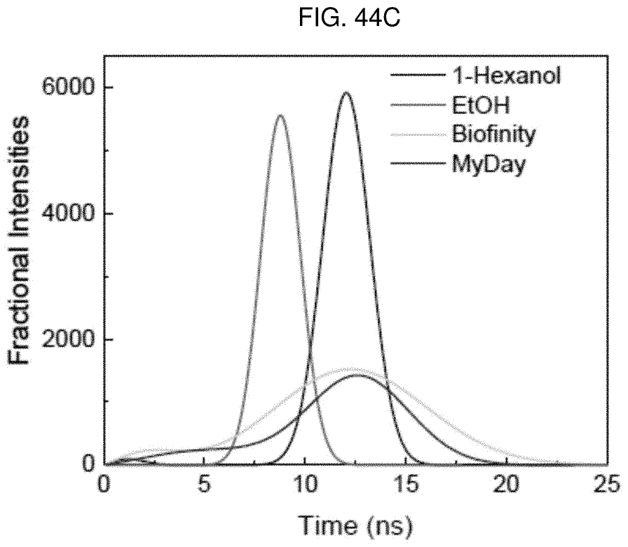

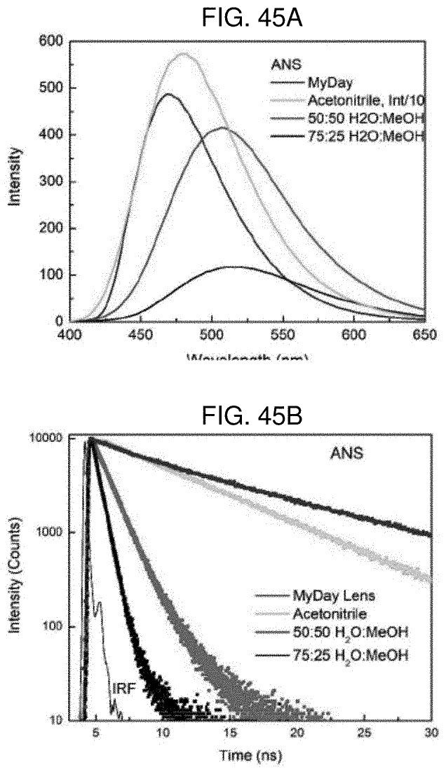

In polar solvents like water, the emission of many fluorophores is shifted to longer wavelengths. In addition, the intensities can be much lower and the lifetimes much shorter. The probe 1-anilino-8-naphthalene sulfonic acid (1,8-ANS) displays a short wavelength emission maximum with high intensity and long lifetime in non-polar environments or in non-polar solvents like acetonitrile. Table 20, below. However, it exhibits relatively weak and long wavelength emission with short fluorescence lifetime in polar or water-containing solvents. See FIG. 44 and FIG. 45.

TABLE 20
Intensity decay analysis of 1,8-ANS in various solvents and contact lenses.

| | τ₁ | τ₂ | | | | | τ_{α} | τ_{f} |
|---|---|---|---|---|---|---|---|---|
| Solvent/Contact Lens | (ns) | (ns) | α₁ | α₂ | f₁ | f₂ | (ns) | (ns) |
| 1-hexanol | 1.77 | 12.02 | 0.08 | 0.92 | 0.01 | 0.99 | 11.18 | 11.88 |
| EtOH | 3.13 | 8.83 | 0.05 | 0.95 | 0.02 | 0.98 | 8.57 | 8.74 |
| Acetonitrile | 7.04 | — | 1.0 | — | 1.0 | — | 7.04 | 7.04 |
| 50/50 MeOH/H₂O | 1.09 | 2.33 | 0.925 | 0.075 | 0.85 | 0.15 | 1.19 | 1.27 |
| 25/75 MeOH/H₂O | 0.52 | 2.36 | 0.996 | 0.004 | 0.98 | 0.02 | 0.52 | 0.54 |
| Biofinity ™ | 0.40 | 11.00 | 0.36 | 0.64 | 0.05 | 0.95 | 8.76 | 11.91 |
| MyDay ™ | 4.01 | 12.79 | 0.421 | 0.578 | 0.20 | 0.80 | 9.70 | 11.97 |

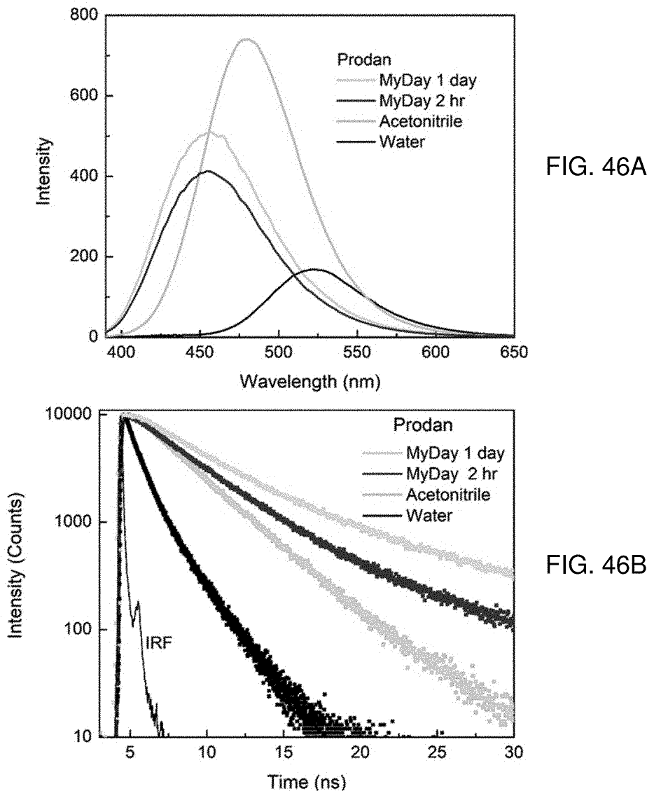

1,8-ANS bound rapidly to the MyDay™ lenses and displayed a short wavelength emission maximum and a long intensity decay time (see FIG. 46), even longer than that of the probe in acetonitrile. The observed spectral properties of 1,8-ANS in lens are consistent with 1,8-ANS properties noticed in a non-polar environment. The probe 6-propionyl-2-dimethylaminonaphthalene (Prodan™), which is also highly sensitive to local polarity, also was used. In contrast to 1,8-ANS, Prodan™ displays good emission in water but with a long emission maximum of 550 nm (see FIG. 44 and FIG. 45; see also Table 21). In MyDay™ lenses the emission maximum is at a shorter wavelength than that in acetonitrile. These results indicated that the MyDay™ lenses contain non-polar regions, comparable to silicone.

TABLE 21
Intensity decay of Prodan ™ in various solvents and contact lenses.

| | τ₁ | τ₂ | | | | | τ_{α} | τ_{f} |
|---|---|---|---|---|---|---|---|---|
| Solvent/Contact Lens | (ns) | (ns) | α₁ | α₂ | f₁ | f₂ | (ns) | (ns) |
| 1-hexanol | 3.40 | — | 1.0 | — | 1.0 | — | 3.40 | 3.40 |
| EtOH | 3.28 | — | 1.0 | — | 1.0 | — | 3.28 | 3.28 |
| Acetonitrile | 3.38 | — | 1.0 | — | 1.0 | — | 3.38 | 3.38 |
| Water | 0.69 | 2.02 | 0.740 | 0.260 | 0.494 | 0.51 | 1.04 | 1.36 |
| Biofinity ™ | 3.98 | — | 1.0 | — | 1.0 | — | 3.98 | 3.98 |
| MyDay ™, 2 hr | 3.81 | 12.15 | 0.94 | 0.07 | 0.82 | 0.18 | 4.36 | 5.34 |
| MyDay ™, 1 day | 4.48 | 14.41 | 0.87 | 0.13 | 0.67 | 0.33 | 5.80 | 7.76 |

### Example 20. Testing of Contact Lenses in Rabbits

A Biofinity™ lens was labeled by immersion in a 1 μM solution of SG-PL in buffer for 10 minutes followed by extensive rinsing. This lens was placed on the eye, ex vivo, of a fresh rabbit head and held in place by the eyelids. The emission from SG-PL was observed using a fiber optic rig.

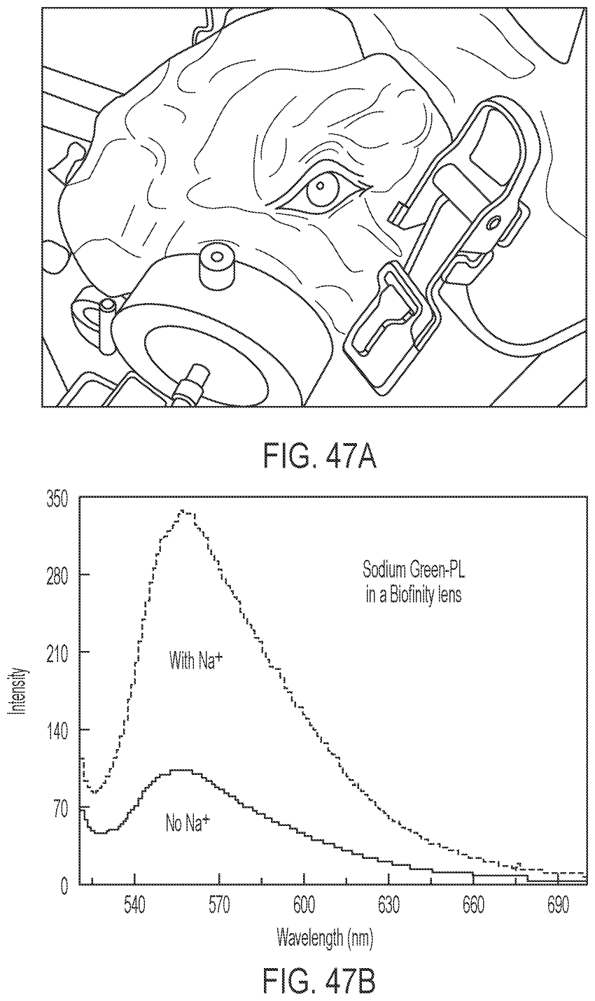

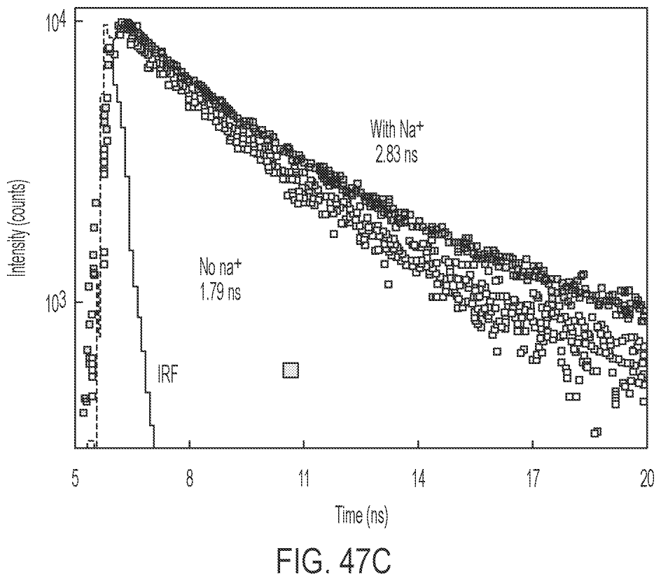

Measurements can be made at a single location in the cornea or eye, or the incident beam can be defocused to obtain a large average signal from the labeled lens. Both single point measurements and lifetime images can be taken to determine if the probes respond to ion concentrations. The emission from SG-PL was readily observed without significant background emission from the rabbit eye. See FIG. 47 for emission spectra and intensity decays. The top panel shows a rabbit in a restrainer used for measurements. Changing the Na⁺ concentration on the eye from 0 to 150 mM resulted in a 3.4-fold increase in emission intensity and an increase in lifetime from 1.79 to 2.83 ns. These spectral changes are in close agreement with the changes observed for SG-PL in a Biofinity™ lens observed in buffer solution.

### REFERENCES

All references listed below and throughout the specification are hereby incorporated by reference in their entirety.
1. Van Haeringen. N. J. (1981). Clinical biochemistry of tears, Sur. Ophthal., 26:84-96.
2. Fullard, R. J., and Tucker, D. L. (1991). Changes in human tear protein levels with progressively increasing stimulus, Invest. Oph. Vis. Sci., 32:2290-2301.
3. von Thun und Hohenstein-Blau, N., Funke, S., and Grus, F. H. (2013). Tears as a source of biomarkers for ocular and systemic diseases, Exp. Eye Res., 126-137.
4. Pflugfelder, S. C. (2003). Anti-inflammatory therapy of dry eye, The Ocular Surface, 1(1):31-36.
5. van Zwieten, R., Wever, R., Hamers, M. N., Weening, R. S., and Roos, D. (1981). Extracellular proton release by stimulated neutrophils, J. Clin. Invest., 66(1):310-313.
6. Bhavsar, A. S., Bhavsar, S. G. and Jain, S. M. (2011). A review on recent advances in dry eye: Pathogenesis and management, Oman J. Ophthalmol., 4(2):50-56.
7. Colligris, B., Alkozi, H. A., and Pintor, J. (2014). Recent developments on dry eye disease treatment compounds, Saudi J. Ophthalmol., 28:19-30.
8. Johnson, M. E., and Murphy, P. J. (2004). Changes in the tear film and ocular surface from dry eye syndrome, Prag. Ret. Eye Res., 23:449-474.
9. Thulasi, P., and Djalilian, A. R. (2017). Update in current diagnostics and therapeutics of dry eye disease, Ophthalmology, 124:S27-S33.
10. Phadatare, S. P., Momin, M., Nighojkar, P., Askarkar, S., and Singh, K. K. (2015). A comprehensive review on dry eye diseases: diagnosis, medical management, recent developments, and future challenges, Adv. Pharmac., article ID 704946, pp 1-12.
11. Tearlab™ Osmalarity Test Card package insert. Tearlab Corp: (2008). September.
12. Versura, P., and Campos, E. C. (2014). Tearlab osmolarity system for diagnosing dry eye, Exp. Rev. Molec. Diagn., 13(2):119-129.
13. Badugu, R., Szmacinski, H., and Lakowicz, J. R. (2020). Fluorescent contact lens for continuous non-invasive measurements of sodium and chloride ion concentrations in tears, in preparation. Analytical Biochemistry (608) 113902.
14. Badugu, R., Lakowicz, J. R., and Geddes, C. D. (2005). Boronic acid fluorescent sensors for monosaccharide signaling based on the 6-methoxy-quinlinium heterocyclic nucleus: progress toward noninvasive and continuous glucose monitoring, Bioorg. Med. Chem. Lett., 15:3974-3977.
15. Versura, P., Profazio, V., and Campos, E. C. (2010). Performance of tear osmolarity compared to previous diagnostic tests for dry eye diseases, Curr. Eye Res., 35(7):553-564.
16. Tomlinson, A., Khanal, S., Ramaesb, K., Diaper, C., and Mcfadyen, A. (2006). Tear film osmolarity: determination of a referent for dry eye diagnosis, Invest. Ophthalmol. Vis. Sci, 47(10):4309-4315.
17. Szczesna-lskander, D. H. (2016). Measurement variability of the Tearlab osmolarity system, Con. Lens Ant. Eye, 39:353-358.
18. Goren. M. B., and Goren, S. B. (1988). Diagnostic tests in patients with symptoms of keratoconjunctivitis sicca, Am. J. Ophthal., 106:570-574.
19. Rao, S., Revathi, R., Durairajan, R., Palanisamy, S., Reddy, S. R., Nainappan, M., and Chandrasekhar, D. (2017). Symposium on dry eye disease management, J. Ophthal. Sci. Res., 55:321-324.
20. Mason, W. T. (Ed.) (1999). Fluorescent and Luminescent Probes for Biological Activity, Academic Press. New York, 647 pp.
21. Javier Ruiz-Ederra, Marc H. Levin, and Verkman, A. S. (2009). In situ fluorescence measurement of tear film [Na.]. [K.], [Cl.] and pH in mice shows marked hypertonicity in Aquaporin-5 deficiency, Invest. Opthalmol. Vis. Sci., 50(5):2132-2138.
22. Foulks, G. N., and Pflugfelder, S. C. (2014). New testing options for diagnosing and grading dry eye disease, Am. J. Ophthalmol., 157:1122-1129.
23. Liu, D. E., Kotsmar, C., Nguyen, F., Sells, T., Taylor, N. O., Prausnitz, J. M., and Radke, C. J. (2013). Macromolecule sorption and diffusion in HEMA/MAA hydrogels, Ind. Eng. Chem. Res., 52:18109-18120.
24. Garrett, Q., Chatelier, R. C., Griesser, H. J., and Milthorpe, B. K. (1988). Effect of charged groups on the adsorption and penetration of proteins onto and into carboxymethylated poly(HEMA) hydrogels, Biomaterials, 19:2175-2186.
25. Tighe, B. (2004). Silicone hydrogel contact lens materials update—Part 1.
26. Weber, G. and Lakowicz, J. R. (1973). Subnanosecond solvent relaxation studies by oxygen quenching of fluorescence, Chem. Phys. Letts., 22:419-423.
27. Schreiner, A. E., and Rose, C. R. (2012). Quantitative imaging of intracellular sodium, Curr. Micro. Con. Adv. Sci. Tech., 119-129.
28. He, H., Mortellaro, M. A, Leiner, M. J. P., Young, S. T., Fraatz, R. J., and Tusa, J. K. (2003) A fluorescent chemosensor for sodium based on photoinduced electron transfer, Anal. Chem., 75:549-555.
29. Nandhikonda, P., Begaye, M. P., and Heagy, M. D. (2009). Highly water-soluble, off-on dual fluorescent probes for sodium and potassium ions, Tethed. Letts., 50:2459-2461.
30. Ohashi, Y., Dogru, M., and Tsubota, K. (2006). Laboratory findings in tear fluid analysis, Clin. Chim. Acta., 369:17-28.
31. Yao, H., Shum, A. J., Cowan, M., Lahdesmaki, I., and Parviz, B. A. (2011). A contact lens with embedded sensor for monitoring tear glucose level, Biosen. Boe/ec., 26:3290-3296.
32. Kim, J., Kim, M., Lee, M-S., Kim, K., Ji, S., Kim, Y-T., Park, J., Na, K., Bae, K-H., Kim, H. K., Bien, F., Lee, C. Y., and Park, J-U (2016). Wearable smart sensor systems integrated on soft contact lenses for wireless ocular diagnostics, Nature Commun., 1-8.
33. Tseng, R. C., Chen, C-C., Hsu, S-M., and Chuang, H-S. (2018). Contact-lens biosensors, Sensors, 18:1-24.
39. International Patent Publication No. WO 2018/017842 A1 to Lakowicz.
34. Kastelan, S., Tomic, M., Salopek-Rabatic, S., and Novak, B. (2013). Diagnostic procedures and management of dry eye, BioMed. Res., 2013:309723-1/6.
35. Scherz, W., Doane, M. G., and Dohlman, C. H. (1974). Tear volume in normal eyes and keratoconjunctivitis sicca, Graefes Arch. Klin. Oph., 141:141-150.
36. Szczesna-Iskander, D. H. (2016). Measurement variability of the TestLab osmolarity system, Con. Lens Ant. Eye, 39:353-358.
37. TearLab™ Osmalarity Test Card package insert. TearLab Corp: (2008). September. 149. Thompson, R. B. (Ed.) Fluorescence Sensors and Biosensors, (2006). Taylor & Francis, New York, 394 pp.
38. Versura, P., and Campos, E. C. (2014). TestLab osmolarity system for diagnosing dry eye, Exp. Rev. Molec., Diagn., 13:119-129.
39. Yetisen, A. K., et al. (2017). Paper-based microfluidic system for tear electrolyte analysis, Lab. On a Chip., 17:1137-1148.
40. Harnett, E. M., Alderman, J., and Wood, T. (2007). The surface energy of various biomaterials coated with adhesion molecules used in cell culture, Colloids Surf Biointer., 15:90-97.
41. Haugland, R. P. (1996). Handbook of Fluorescent Probes and Research Chemicals, 6th ed. Molecular Probes Inc., Eugene, Oreg., pp 503-584.
42. Szmacinski, H., and Lakowicz, J. R. (1997). Sodium green as a potential probe for intracellular sodium imaging based on fluorescence lifetime, Anal. Biochem., 250:131-138.
43. Schreiner, A. E., and Rose, C. R. (2012). Quantitative imaging of intracellular sodium, Curr. Micro. Con. Adv. Sci. Tech., 119-129.
44. Doussineau, T., Schulz, A., Lapresta-Femandez, S., Moro, A., Korsten, S., Trupp, S., and Mohr, G. J. (2010). On the design of fluorescent ratiometric nanosensors, Chem. Eur. J., 16:10290-10299.
45. Ohashi, Y., Dogru, M., and Tsubota, K. (2006). Laboratory findings in tear fluid analysis, Clin. Chim. Acta., 369:17-28.
46. Geddes, C. D., Apperson, K., Karolin, J., and Birch, D. J. S. (2001). Chloride-sensitive fluorescent indicators, Anal. Biochem., 293:60-66.
47. Arosio, D., and Ratto, G. M. (2014). Twenty years of fluorescence imaging of intracellular chloride, Front. Cell. Neurosci., 8:1-2.
48. Lakowicz, J. R., Principles of Fluorescence Spectroscopy, 3^{rd} edition, 2006. Springer Science+Business Media, LLC. Chapter 19, pages 623-641.
49. Scherz, W., Doane, M. G., and Dohlman, C. H. (1974). Tear volume in normal eyes and keratoconjunctivitis sicca, Graefes Arch. Klin. Ophthalmol., 141(2):141-150.
50. Fassett, J. D.• and Paulsen, P. J. (1989). Isotope dilution mass spectrometry for accurate elemental analysis, Anal. Chem., 61:643A-649A.
51. Doussineau, T., Schulz, A., Lapresta-Femandez, A., Moro, A, Kersten, S., Trupp, S., and Mohr, G. J. (2010). On the design of fluorescent ratiometric nanosensors, Chem. Eur. J., 16:10290-10299.
52. Mattioli Della Rocca, F., Nedbal, J., Tyndall, D., Krstajic, N., Day-Uei Li, D., Arneer-6eg, S. M. and Henderson, R. K. (2016). Real-time fluorescence lifetime actuation for cell sorting using a CMOS SPAD silicon photomultiplier, Optics Letts., 41(4):673-676.
53. Rae, B. R., Muir, K. R., Gong, Z., McKendry, J., Girkin, J. M., Gu, E., Renshaw, D., Dawson, M. D., and Henderson, R. K. (2009). A CMOS time-resolved fluorescence lifetime analysis micro-system, Sensors, 9:9255-9274.
54. Badugu, R., Lakowicz, J. R., and Geddes, C. D. (2003). A glucose sensing contact lens: A non-invasive technique for continuous physiological glucose monitoring, J. Fluores., 13:371-374.
55. Badugu, R., Jeng, B. H., Reece, E. A., and Lakowicz, J. R. (2018). Contact lens to measure individual ion concentrations in tears and applications to dry eye disease, Anal. Biochem., 542:84-94.
56. Badugu, R., Reece, E. A., and Lakowicz, J. R. (2018). Glucose-sensitive silicone hydrogel contact lens toward tear glucose monitoring, Biomed. Opt., 23:1-5.
57. Schirmer, 0. (1903). Albrecht von Graefe's archive for clinical and experimental ophthalmology. Albrecht von Graefes Arch. Oph., 56:197-291.
58. Wright, J. C., and Meger, G. E. (1962). A review of the Schimier test for tear production, Arch. Ophthal., 67:564-565.
59. Fullord, R. J., and Tucker, D. L. (1991). Changes in human tear protein levels with progressively increasing stimulus, lnves. Ophthalmol. Vis. Sci., 32(8):2290-2301.
60. Stuchell, R. N., Feldman, J. J., Forris, R. L., and Mondel, I. D. (1984). The effect of collection technique on tear composition, lnves. Ophthalmol. Vis. Sci., 25:374-377.
61. Jones, L., Downie, L., Korb, D., et al. (2017). TFOS DEWS II management and therapy report, The Ocular Sur., 15:575-628.

1. A fluorescent probe compound comprising at least one fluorophore that is sensitive to an electrolyte analyte selected from the group consisting of sodium, potassium, chloride, calcium, magnesium, and hydrogen, and that contains a hydrophilic region and a hydrophobic moiety,
wherein the excitation wavelength of the fluorophore is from about 280 nm to about 750 nm; and wherein the hydrophobic moiety is configured to allow the fluorescent probe to bind non-covalently to a silicone hydrogel material.

2. The fluorescent probe compound of claim 1, wherein the hydrophilic region is native to the fluorophore.

3. The fluorescent probe compound of claim 1, wherein the fluorophore has been modified to contain a hydrophilic region.

4. The fluorescent probe compound of claim 1, further comprising one or more linkers or spacers.

5. The fluorescent probe compound of claim 1, wherein the analyte is selected from the group consisting of sodium ion, chloride ion, potassium, hydrogen ion, calcium ion, magnesium ion.

6. The fluorescent probe compound of claim 1, wherein the fluorophore is selected from the group consisting of sodium green, SBFI, PBFI, CD 222, Fura-2, Indo-1, calcium green, and magnesium orange.

7. The fluorescent probe compound of claim 1, wherein the hydrophobic moiety is selected from the group consisting of an alkyl chain having 12 or more carbon atoms and an optional terminal amine group, poly-L-lysine with a molecular weight of about 70 kDa to about 150 kDa, lyso phosphatidyl ethanolamine, —NH₂—(CH₂)ₙ—CH₃ where n is 12-25, (—CH₂)ₙ—CH═CH₂ where n is 12-25, a saturated or unsaturated fatty acid chain having about 12-25 carbon atoms, phytyl groups, lysophospholipid, cholesterol, and mixtures thereof.

8. A silicone hydrogel contact lens comprising at least one fluorescent probe compound of claim 1, bound to the silicone hydrogel contact lens.

9. A silicone hydrogel contact lens comprising at least one fluorescent probe compound of claim 1, wherein the fluorescent probe compound is bound to the silicone hydrogel contact lens non-covalently.

10. A silicone hydrogel contact lens of claim 9, wherein the at least one fluorescent probe compound binds to the silicone hydrogel at the water-silicone interface and/or nonpolar areas.

11. A silicone hydrogel contact lens of claim 8, wherein the at least one fluorescent probe compound is not removed from the contact lens by exposure to tears for at least 1 day.

12. A silicone hydrogel contact lens of claim 8, wherein the at least one fluorescent probe compound is not significantly removed from the contact lens by exposure to tears for at least 7 days.

13. A silicone hydrogel contact lens of claim 8, wherein a plurality of different fluorescent probe compounds are bound to the contact lens.

14. The silicone hydrogel contact lens of claim 13, wherein the plurality of different fluorescent probe compounds are bound to different discrete areas of the contact lens.

15. The silicone hydrogel contact lens of claim 8 which further comprises comfilcon A or stenfilcon A.

16. A system comprising the silicon hydrogel contact lens of claim 8 and a wavelength ratiometric sensor.

17. A method of measuring electrolytes in basal tears in a subject in need without perturbation of the tear composition, comprising:
(a) placing the contact lens of claim 8 on the eye of the subject;
(b) waiting at least about 10 minutes;
(c) exposing the contact lens to light at the excitation wavelength of the at least one fluorescent probe;
(d) detecting the emitted light from the contact lens; and
(e) recording the wavelength-radiometric measurements or intensity decays of the emitted light.
